(19) United States
(12) Patent Application Publication
(10) Pub. No.: US 2025/0352514 A1
(43) Pub. Date: Nov. 20, 2025

(54) SALT FORMS OF BIS(3-(2-(DIMETHYLAMINO)ETHYL)-1 H-INDOL-4-YL) 3,3'¬OXYDIPROPIONATE

(71) Applicant: Compass Pathfinder Limited, Altrincham (GB)

(72) Inventors: John Richard MORPHY, Altrincham (GB); Paul Stanley BURY, Harlow (GB); Niall Anthony WAGSTAFF, Harlow (GB); Paul Stuart HINCHLIFFE, Harlow (GB); Susan Elizabeth OSBOURN, Harlow (GB)

(21) Appl. No.: 19/210,063

(22) Filed: May 16, 2025

Related U.S. Application Data
(60) Provisional application No. 63/649,108, filed on May 17, 2024.
(60) Provisional application No. 63/761,491, filed on Feb. 21, 2025.

(51) Int. Cl. A61K 31/4045; C07D 209/16

(52) U.S. Cl. CPC A61K 31/4045; C07D 209/16

(57) ABSTRACT

The present disclosure provides salt forms of Compound 1,

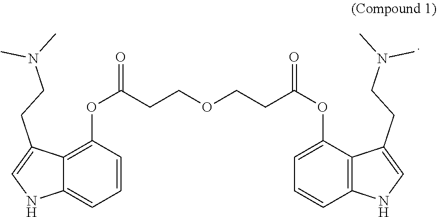

### CROSS-REFERENCE TO RELATED APPLICATIONS

This application claims priority to U.S. Provisional Application No. 63/649,108, filed May 17, 2024, and U.S. Provisional Application No. 63/761,491, filed Feb. 21, 2025, the disclosures of which are incorporated by reference in their entireties for all purposes.

### BACKGROUND

Over 50% of adults in the United States will be diagnosed with a psychiatric disorder at some point in their lifetime. Nearly 1 in 5 suffer from mental illness, and nearly 1 in 25 are afflicted with severe mental illness, such as major depression, schizophrenia, or bipolar disorder. Psychedelics show promising activity in treating mental illness.

Crystallization of an active pharmaceutical ingredient can offer benefits for physiochemical properties. Such physicochemical properties include solubility, in vivo bioavailability, stability, melting point, flow properties, hygroscopicity, among others. Therefore, crystalline forms can be advantageous for pharmaceutical products. Thus, there is a need for crystalline forms of psychedelics to treat psychiatric disorders.

### SUMMARY

The present disclosure relates to pharmaceutically acceptable salt forms of Compound 1:

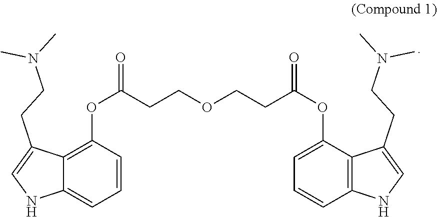

In embodiments, the salt is a monofumarate salt of Compound 1, a sesquifumarate salt of compound 1, bis-formate salt of compound 1, succinate salt of compound 1, or L-tartrate salt of compound 1.

In embodiments, the monofumarate salt of Compound 1 is crystalline. In embodiments, the monofumarate salt of Compound 1 is Form 1, Form 2, Form 3, Form 4, Form 5, Form 6, Form 7, Form 8, or Form 9.

In embodiments, the monofumarate salt of Compound 1 is Form 1. In embodiments, the monofumarate salt is characterized by an X-ray powder diffraction (XRPD) pattern having peaks at 12.8±0.2, 18.7±0.2, and 23.2±0.2 °2θ. In embodiments, the monofumarate salt of Compound 1 is further characterized by peaks in an XRPD pattern at 7.2±0.2, 10.4±0.2, 17.9±0.2, 19.3±0.2, and 21.4±0.2 °2θ. In embodiments, the monofumarate salt of Compound 1 is characterized by an XRPD pattern having peaks at 5.3±0.2, 7.2±0.2, 7.7±0.2, 8.9±0.2, 10.4±0.2, 11.3±0.2, 12.8±0.2, 13.7±0.2, 14.4±0.2, 14.7±0.2, 15.4±0.2, 16.5±0.2, 16.9±0.2, 17.3±0.2, 17.9±0.2, 18.7±0.2, 19.3±0.2, 19.8±0.2, 20.8±0.2, 21.0±0.2, 21.4±0.2, 21.8±0.2, 22.8±0.2, 23.2±0.2, 24.5±0.2, 24.8±0.2, 25.7±0.2, 26.4±0.2, 27.4±0.2, 28.6±0.2, 29.0±0.2, 29.5±0.2, 30.2±0.2, 30.6±0.2, 31.3±0.2, 32.3±0.2, and 32.7±0.2 °2θ. In embodiments, the monofumarate salt of Compound 1 is characterized by an XRPD pattern substantially similar to FIG. 3B.

In embodiments, the monofumarate salt of Compound 1 is characterized by a Differential Scanning Calorimetry (DSC) thermogram comprising an endothermic event between 88±° C. to 142±5° C., 144±5° C. to 155±5° C., and 156±5° C. to 174±5° C. In embodiments, the monofumarate salt of Compound 1 is characterized by a DSC comprising an exothermic event between 175±5° C. to 212±5° C. In embodiments, the monofumarate salt of Compound 1 is characterized by a DSC thermogram substantially similar to FIG. 3C.

In embodiments, the monofumarate salt of Compound 1 is characterized by about a 4.8% weight loss from 36±5° C. to 180±5° C. as determined by thermal gravimetric analysis (TGA). In embodiments, the monofumarate salt is characterized by a thermogravimetric thermogram substantially similar to FIG. 3D.

In embodiments, the monofumarate salt of Compound 1 is Form 2. In embodiments, the monofumarate salt of Compound 1 is characterized by an XRPD pattern having peaks at 12.7±0.2, 18.0±0.2, and 20.7±0.2 °2θ. In embodiments, the monofumarate salt of Compound 1 is further characterized by peaks in an XRPD pattern at 10.4±0.2, 16.7±0.2, 22.0 0.2, 24.0 0.2, and 25.5±0.2 °2θ. In embodiments, the monofumarate salt of Compound 1 is characterized by an XRPD pattern having peaks at characterized by an XRPD pattern having peaks at 8.7±0.2, 9.6±0.2, 10.4±0.2, 10.9±0.2, 12.7±0.2, 14.6±0.2, 15.8±0.2, 16.7±0.2, 18.0±0.2, 18.6±0.2, 19.1±0.2, 19.3±0.2, 19.7±0.2, 20.0±0.2, 20.7±0.2, 21.4±0.2, 22.0±0.2, 22.9±0.2, 23.4±0.2, 24.0±0.2, 25.5±0.2, 26.3±0.2, 26.6±0.2, 27.4±0.2, 29.3±0.2, 30.3±0.2, 33.8±0.2, 34.9±0.2, and 37.7±0.2 °2θ. In embodiments, the monofumarate salt of Compound 1 is characterized by an XRPD pattern substantially similar to FIG. 3H.

In embodiments, the monofumarate salt of Compound 1 is Form 3. In embodiments, the monofumarate salt of Compound 1 is characterized by an XRPD pattern having peaks at 16.8±0.2, 17.7±0.2, and 19.2±0.2 °2θ. In embodiments, the monofumarate salt of Compound 1 is further characterized by peaks in an XRPD pattern at 19.8±0.2, 21.6±0.2, 23.7±0.2, 24.5±0.2, and 24.9±0.2 °2θ. In embodiments, the monofumarate salt of Compound 1 is characterized by an XRPD pattern having peaks at 7.8±0.2, 8.7±0.2, 9.9±0.2, 10.2±0.2, 10.7±0.2, 11.3±0.2, 11.9±0.2, 12.1±0.2, 12.5±0.2, 13.1±0.2, 14.4±0.2, 15.2±0.2, 16.2±0.2, 16.8±0.2, 17.7±0.2, 18.3±0.2, 19.2±0.2, 19.8±0.2, 21.0±0.2, 21.6±0.2, 23.0±0.2, 23.7±0.2, 24.0±0.2, 24.5±0.2, 24.9±0.2, 25.2±0.2, 26.5±0.2, 27.2±0.2, 27.9±0.2, 29.1±0.2, 29.5±0.2, 29.9±0.2, 31.9±0.2, 32.3±0.2, 34.0±0.2, and 35.1±0.2 °2θ. In embodiments, the monofumarate salt of Compound 1 is characterized by an XRPD pattern substantially similar to FIG. 3I.

In embodiments, the monofumarate salt of Compound 1 is Form 4. In embodiments, the monofumarate salt of Compound 1 is characterized by an XRPD pattern having peaks at 8.2±0.2, 11.3±0.2, and 21.0±0.2 °2θ. In embodiments, the monofumarate salt of Compound 1 is further characterized by peaks in an XRPD pattern at 5.7±0.2, 9.3±0.2, 16.9±0.2, 18.5±0.2, and 19.7±0.2 °2θ. In embodiments, the monofumarate salt of Compound 1 is characterized by an XRPD pattern having peaks at 5.7±0.2, 7.2±0.2, 7.6±0.2, 8.2±0.2, 8.8±0.2, 9.3±0.2, 10.2±0.2, 10.5±0.2, 11.3±0.2, 12.3±0.2, 12.9±0.2, 13.2±0.2, 13.6±0.2, 14.5±0.2, 15.2±0.2, 15.8±0.2, 16.2±0.2, 16.4±0.2, 16.9±0.2, 17.3±0.2, 17.6±0.2, 18.5±0.2, 18.9±0.2, 19.4±0.2, 19.7±0.2, 20.4±0.2, 21.0±0.2, 21.4±0.2, 21.9±0.2, 22.6±0.2, 23.1±0.2, 23.7±0.2, 24.8±0.2, 25.3±0.2, 25.4±0.2, 26.3±0.2, 27.0±0.2, 28.2±0.2, 29.1±0.2, 30.4±0.2, 32.0±0.2, and 33.9±0.2 °2θ. In embodiments, the monofumarate salt of Compound 1 is characterized by an XRPD pattern substantially similar to FIG. 3J.

In embodiments, the monofumarate salt of Compound 1 is Form 5. In embodiments, the monofumarate salt of Compound 1 is characterized by an XRPD pattern having peaks at 8.7±0.2, 19.7±0.2, and 23.5±0.2 °2θ. In embodiments, the monofumarate salt of Compound 1 is further characterized by peaks in an XRPD pattern at 14.4±0.2, 18.7±0.2, 19.2±0.2, 21.8±0.2, and 23.2±0.2 °2θ. In embodiments, the monofumarate salt of Compound 1 is characterized by an XRPD pattern having peaks at 7.1±0.2, 7.8±0.2, 8.7±0.2, 9.5±0.2, 10.5±0.2, 11.1±0.2, 11.6±0.2, 12.2±0.2, 12.8±0.2, 14.4±0.2, 15.6±0.2, 16.4±0.2, 16.8±0.2, 17.4±0.2, 17.7±0.2, 18.7±0.2, 19.2±0.2, 19.7±0.2, 20.0±0.2, 20.8±0.2, 21.1±0.2, 21.4±0.2, 21.8±0.2, 22.2±0.2, 22.8±0.2, 23.2±0.2, 23.5±0.2, 24.6±0.2, 25.2±0.2, 26.4±0.2, 26.7±0.2, 28.2±0.2, 29.0±0.2, 33.3±0.2, and 34.8±0.2 °2θ. In embodiments, the monofumarate salt of Compound 1 is characterized by an XRPD pattern substantially similar to FIG. 3K.

In embodiments, the monofumarate salt of Compound 1 is Form 6. In embodiments, the monofumarate salt of Compound 1 is characterized by an XRPD pattern having peaks at 7.3±0.2, 10.6±0.2, and 19.8±0.2 °2θ. In embodiments, the monofumarate salt of Compound 1 is further characterized by peaks in an XRPD pattern at 13.9±0.2, 18.0±0.2, 20.4±0.2, 23.1±0.2, and 23.6±0.2 °2θ. In embodiments, the monofumarate salt of Compound 1 is characterized by an XRPD pattern having peaks at 7.3±0.2, 8.0 0.2, 8.4±0.2, 8.9±0.2, 10.1±0.2, 10.6±0.2, 11.6±0.2, 12.1±0.2, 12.4±0.2, 13.7±0.2, 13.9±0.2, 14.6±0.2, 15.1±0.2, 15.5±0.2, 15.8±0.2, 16.1±0.2, 16.3±0.2, 18.0±0.2, 18.3±0.2, 18.8±0.2, 19.2±0.2, 19.8±0.2, 20.4±0.2, 20.9±0.2, 21.8±0.2, 22.2±0.2, 22.5±0.2, 23.1±0.2, 23.6±0.2, 24.0±0.2, 24.6±0.2, 25.1±0.2, 25.7±0.2, 26.1±0.2, 26.9±0.2, 28.3±0.2, 29.2±0.2, 31.0±0.2, 31.6±0.2, 33.2±0.2, 34.9±0.2, and 36.8±0.2 °2θ. In embodiments, the monofumarate salt of Compound 1 is characterized by an XRPD pattern substantially similar to FIG. 3L.

In embodiments, the monofumarate salt of Compound 1 is Form 7. In embodiments, the monofumarate salt of Compound 1 is characterized by an XRPD pattern having peaks at 18.7±0.2, 23.6±0.2, and 25.2±0.2 °2θ. In embodiments, the monofumarate salt of Compound 1 is further characterized by peaks in an XRPD pattern at 8.7±0.2, 10.5±0.2, 17.2±0.2, 19.7±0.2, and 21.4±0.2 °2θ. In embodiments, the monofumarate salt of Compound 1 is characterized by an XRPD pattern having peaks at 5.3±0.2, 7.2±0.2, 8.7±0.2, 8.9±0.2, 10.2±0.2, 10.5±0.2, 11.5±0.2, 11.8±0.2, 12.2±0.2, 12.8±0.2, 14.1±0.2, 15.2±0.2, 16.5±0.2, 17.2±0.2, 17.9±0.2, 18.2±0.2, 18.7±0.2, 19.4±0.2, 19.7±0.2, 20.8±0.2, 21.1±0.2, 21.4±0.2, 21.9±0.2, 23.2±0.2, 23.6±0.2, 24.0±0.2, 24.9±0.2, 25.2±0.2, 26.4±0.2, 27.5±0.2, 29.2±0.2, 30.3±0.2, and 38.1±0.2 °2θ. In embodiments, the monofumarate salt of Compound 1 is characterized by an XRPD pattern substantially similar to FIG. 3M.

In embodiments, the monofumarate salt of Compound 1 is Form 8. In embodiments, the monofumarate salt of Compound 1 is characterized by an XRPD pattern having peaks at 5.6±0.2, 11.2±0.2, and 19.7±0.2 °2θ. In embodiments, the monofumarate salt of Compound 1 is further characterized by peaks in an XRPD pattern at 9.3±0.2, 16.8±0.2, 18.6±0.2, 21.1±0.2, and 23.5±0.2 °2θ. In embodiments, the monofumarate salt of Compound 1 is characterized by an XRPD pattern having peaks at 5.6±0.2, 7.1±0.2, 8.1±0.2, 8.7±0.2, 9.3±0.2, 10.4±0.2, 11.2±0.2, 12.7±0.2, 13.2±0.2, 14.4±0.2, 15.0±0.2, 15.8±0.2, 16.8±0.2, 18.6±0.2, 19.3±0.2, 19.7±0.2, 20.8±0.2, 21.1±0.2, 21.8±0.2, 22.5±0.2, 23.2±0.2, 23.5±0.2, 25.1±0.2, 29.1±0.2, and 29.5±0.2 °2θ. In embodiments, the monofumarate salt of Compound 1 is characterized by an XRPD pattern substantially similar to FIG. 3N.

In embodiments, the monofumarate salt of Compound 1 is Form 9. In embodiments, the monofumarate salt of Compound 1 is characterized by an XRPD pattern having peaks at 10.8±0.2, 16.2±0.2, and 21.6±0.2 °2θ. In embodiments, the monofumarate salt of Compound 1 is further characterized by peaks in an XRPD pattern at 5.5±0.2, 12.1±0.2, 17.7±0.2, 18.2±0.2, and 22.7±0.2 °2θ. In embodiments, the monofumarate salt of Compound 1 is characterized by an XRPD pattern having peaks at 5.5±0.2, 6.1±0.2, 8.2±0.2, 8.8±0.2, 9.6±0.2, 10.0±0.2, 10.8±0.2, 11.4±0.2, 12.1±0.2, 12.9±0.2, 13.2±0.2, 14.6±0.2, 16.2±0.2, 17.0±0.2, 17.7±0.2, 18.2±0.2, 19.1±0.2, 19.9±0.2, 20.4±0.2, 20.7±0.2, 21.6±0.2, 22.7±0.2, 23.5±0.2, 24.2±0.2, 25.3±0.2, 27.9±0.2, 28.6±0.2, 29.5±0.2, 32.5±0.2, and 33.3±0.2 °2θ. In embodiments, the monofumarate salt of Compound 1 is characterized by an XRPD pattern substantially similar to FIG. 3O.

In embodiments, the monofumarate salt of Compound 1 has a chemical purity of greater than about 90%, by weight, as determined by HPLC analysis. In embodiments, the monofumarate salt of Compound 1 has a chemical purity of greater than about 95%, by weight, as determined by HPLC analysis. In embodiments, the monofumarate salt of Compound 1 has a chemical purity of greater than about 99.5%, by weight, as determined by HPLC analysis.

In embodiments, the sesquifumarate salt of Compound 1 is crystalline. In embodiments, the sesquifumarate salt of Compound 1 is characterized by an XRPD pattern having peaks at 8.7±0.2, 19.7±0.2, and 23.6±0.2 °2θ. In embodiments, the sesquifumarate salt of Compound 1 is further characterized by peaks in an XRPD pattern at 14.4±0.2, 21.8±0.2, and 22.2±0.2 °2θ. In embodiments, the sesquifumarate salt of Compound 1 is characterized by an XRPD pattern having peaks at 7.9±0.2, 8.7±0.2, 9.6±0.2, 11.1±0.2, 11.6±0.2, 12.3±0.2, 14.0±0.2, 14.4±0.2, 15.6±0.2, 16.3±0.2, 16.8±0.2, 17.2±0.2, 17.4±0.2, 17.8±0.2, 18.0±0.2, 18.4±0.2, 18.7±0.2, 19.2±0.2, 19.7±0.2, 20.1±0.2, 20.8±0.2, 21.1±0.2, 21.4±0.2, 21.8±0.2, 22.2±0.2, 22.5±0.2, 22.9±0.2, 23.2±0.2, 23.6±0.2, 24.3±0.2, 24.6±0.2, 25.3±0.2, 26.4±0.2, 26.8±0.2, 27.4±0.2, 28.2±0.2, 29.0±0.2, 29.5±0.2, 29.7±0.2, 30.1±0.2, 30.4±0.2, 30.9±0.2, 31.6±0.2, 33.2±0.2, 34.0±0.2, 34.7±0.2, 34.9±0.2, 35.4±0.2, 38.4, 39.0±0.2, and 39.9±0.2 and °2θ. In embodiments, the sesquifumarate salt of Compound 1 is characterized by an XRPD pattern substantially similar to FIG. 4A.

In embodiments, the sesquifumarate salt of Compound 1 is characterized by a DSC thermogram comprising an endothermic event at 168±5° C. In embodiments, the sesquifumarate salt of Compound 1 is characterized by a DSC comprising an exothermic event between 176±5° C. to 221±5° C. In embodiments, the sesquifumarate salt of Compound 1 is characterized by a DSC thermogram substantially similar to FIG. 4C.

In embodiments, the sesquifumarate salt of Compound 1 is characterized by about a 2% weight loss from 37±5° C. to 150±5° C. as determined by TGA. In embodiments, the sesquifumarate salt of Compound 1 characterized by a thermogravimetric thermogram substantially similar to FIG. 4D.

In embodiments, the sesquifumarate salt of Compound 1 has a chemical purity of greater than about 90%, by weight, as determined by HPLC analysis. In embodiments, the sesquifumarate salt of Compound 1 has a chemical purity of greater than about 95%, by weight, as determined by HPLC analysis. In embodiments, the sesquifumarate salt of Compound 1 has a chemical purity of greater than about 99.5%, by weight, as determined by HPLC analysis.

In embodiments, the sesquifumarate salt of Compound 1 is a hydrate.

In embodiments, the succinate salt of Compound 1 is crystalline. In embodiments, the succinate salt of Compound 1 is characterized by an XRPD pattern having peaks at 19.7±0.2, 22.1±0.2, and 23.1±0.2 °2θ. In embodiments, the succinate salt of Compound 1 is further characterized by peaks in an XRPD pattern at 8.7±0.2, 14.3±0.2, 16.8±0.2, 18.6±0.2, 19.0 0.2, and 20.2±0.2 °2θ. In embodiments, the succinate salt of Compound 1 is characterized by an XRPD pattern having peaks at 7.8±0.2, 8.7±0.2, 9.7±0.2, 10.8±0.2, 11.6±0.2, 12.0±0.2, 12.5±0.2, 13.9±0.2, 14.3±0.2, 15.6±0.2, 15.9±0.2, 16.5±0.2, 16.8±0.2, 17.4±0.2, 18.0±0.2, 18.6±0.2, 19.0±0.2, 19.7±0.2, 20.2±0.2, 20.7±0.2, 21.2±0.2, 21.7±0.2, 22.1±0.2, 22.3±0.2, 23.1±0.2, 23.6±0.2, 23.9±0.2, 24.6±0.2, 24.9±0.2, 25.3±0.2, 26.2±0.2, 26.9±0.2, 27.6±0.2, 28.1±0.2, 28.3±0.2, 29.3±0.2, 30.2±0.2, 30.4±0.2, 33.2±0.2, 34.6±0.2, 35.1±0.2, 35.7±0.2, 38.0±0.2, 38.7±0.2, and 39.3±0.2 and °2θ. In embodiments, the succinate salt of Compound 1 is characterized by an XRPD pattern substantially similar to FIG. 5A.

In embodiments, the succinate salt of Compound 1 is characterized by a DSC thermogram comprising an endothermic event between 92±5° C. to 129±5° C. In embodiments, the succinate salt of Compound 1 is characterized by a DSC thermogram comprising an endothermic onset at 99±5° C. In embodiments, the succinate salt of Compound 1 is characterized by a DSC thermogram substantially similar to FIG. 5B.

In embodiments, the succinate salt of Compound 1 is characterized by about a 2% weight loss from 37±5° C. to 128±5° C. as determined by TGA. In embodiments, the succinate salt of Compound 1 characterized by a thermogravimetric thermogram substantially similar to FIG. 5C.

In embodiments, the succinate salt of Compound 1 has a chemical purity of greater than about 90%, by weight, as determined by HPLC analysis. In embodiments, the succinate salt of Compound 1 has a chemical purity of greater than about 95%, by weight, as determined by HPLC analysis. In embodiments, the succinate salt of Compound 1 has a chemical purity of greater than about 99.5%, by weight, as determined by HPLC analysis.

In embodiments, the L-tartrate salt of Compound 1 is crystalline. In embodiments, the L-tartrate salt of Compound 1 is characterized by an XRPD pattern substantially similar to FIG. 6A.

In embodiments, the L-tartrate salt of Compound 1 is characterized by a DSC thermogram comprising an endothermic onset at 170±5° C. In embodiments, the L-tartrate salt of Compound 1 is characterized by a DSC thermogram substantially similar to FIG. 6B.

In embodiments, the L-tartrate salt of Compound 1 is crystalline. In embodiments, the L-tartrate salt of Compound 1 is characterized by an XRPD pattern having peaks at 16.0±0.2, 20.1±0.2, and 23.0±0.2 °2θ. In embodiments, the L-tartrate salt of Compound 1 is further characterized by peaks in an XRPD pattern at 7.5±0.2, 14.6±0.2, 19.6±0.2, 23.0±0.2 and 23.4±0.2 °2θ. In embodiments, the L-tartrate salt of Compound 1 is characterized by an XRPD pattern having peaks at 5.5±0.2, 7.5±0.2, 8.0±0.2, 10.5±0.2, 11.3±0.2, 11.6±0.2, 12.4±0.2, 12.7±0.2, 14.6±0.2, 16.0±0.2, 16.5±0.2, 16.8±0.2, 17.5±0.2, 18.9±0.2, 19.6±0.2, 20.1±0.2, 20.6±0.2, 21.2±0.2, 21.7±0.2, 23.4±0.2, 23.0±0.2, 23.4±0.2, 23.8±0.2, 25.5±0.2, 27.5±0.2, 29.1±0.2, 30.8±0.2, and 34.8±0.2 °2θ.

In embodiments, the L-tartrate salt of Compound 1 has a chemical purity of greater than about 90%, by weight, as determined by HPLC analysis. In embodiments, the L-tartrate salt of Compound 1 has a chemical purity of greater than about 95%, by weight, as determined by HPLC analysis. In embodiments, the L-tartrate salt of Compound 1 has a chemical purity of greater than about 99.5%, by weight, as determined by HPLC analysis.

In embodiments, the present disclosure provides a hydrochloride salt of Compound 1. In embodiments, the hydrochloride salt of Compound 1 is crystalline. In embodiments, the hydrochloride salt of Compound 1 is characterized by an XRPD pattern having peaks at 20.3±0.2, 20.7±0.2, and 23.3±0.2 °2θ. In embodiments, the hydrochloride salt of Compound 1 is further characterized by peaks in an XRPD pattern at 9.9±0.2, 16.1±0.2, 17.6±0.2, 19.7±0.2, and 26.7±0.2 °2θ. In embodiments, the hydrochloride salt of Compound 1 is characterized by an XRPD pattern having peaks at 7.5±0.2, 7.8±0.2, 9.9±0.2, 10.4±0.2, 11.2±0.2, 13.3±0.2, 13.7±0.2, 15.4±0.2, 16.1±0.2, 16.8±0.2, 17.6±0.2, 17.9±0.2, 18.2±0.2, 18.7±0.2, 19.7±0.2, 20.3±0.2, 20.7±0.2, 21.1±0.2, 21.4±0.2, 21.9±0.2, 22.4±0.2, 22.7±0.2, 23.3±0.2, 23.6±0.2, 23.9±0.2, 24.9±0.2, 25.2±0.2, 26.3±0.2, 26.7±0.2, 27.4±0.2, 28.2±0.2, 28.9±0.2, 29.7±0.2, 31.0±0.2, 31.5±0.2, 32.5±0.2, 33.0±0.2, 33.5±0.2, 33.8±0.2, 35.3±0.2, 36.2±0.2, 36.7±0.2, 37.1±0.2, 37.9±0.2, 38.8±0.2, 39.3±0.2, 40.0±0.2, and 40.8±0.2 °2θ. In embodiments, the hydrochloride salt of Compound 1 is characterized by an XRPD pattern substantially similar to FIG. 8A.

In embodiments, the hydrochloride salt of Compound 1 is characterized by an XRPD pattern having peaks at 20.2±0.2, 22.0±0.2, and 22.7±0.2 °2θ. In embodiments, the hydrochloride salt of Compound 1 is further characterized by peaks in an XRPD pattern at 13.1±0.2, 16.3±0.2, 18.8±0.2, 20.4±0.2, and 27.0±0.2 °2θ. In embodiments, the hydrochloride salt of Compound 1 is characterized by an XRPD pattern having peaks at 7.6±0.2, 7.9±0.2, 9.8±0.2, 10.7±0.2, 10.9±0.2, 13.1±0.2, 13.7±0.2, 14.3±0.2, 14.6±0.2, 15.7±0.2, 15.9±0.2, 16.3±0.2, 16.7±0.2, 17.2±0.2, 18.8±0.2, 19.6±0.2, 19.9±0.2, 20.2±0.2, 20.4±0.2, 20.8±0.2, 21.1±0.2, 21.4±0.2, 22.0±0.2, 22.7±0.2, 22.9±0.2, 23.9±0.2, 24.5±0.2, 24.9±0.2, 26.3±0.2, 27.0±0.2, 27.2±0.2, 27.8±0.2, 28.2±0.2, 28.4±0.2, 29.3±0.2, 29.5±0.2, 30.6±0.2, 31.7±0.2, 32.0±0.2, 32.9±0.2, 34.3±0.2, 35.7±0.2, 36.6±0.2, 37.2±0.2, 37.7±0.2, 37.9±0.2, 38.8±0.2, and 40.5±0.2 °2θ. In embodiments, the hydrochloride salt of Compound 1 is characterized by an XRPD pattern substantially similar to FIG. 8B.

In embodiments, the hydrochloride salt of Compound 1 has a chemical purity of greater than about 90%, by weight, as determined by HPLC analysis. In embodiments, the hydrochloride salt of Compound 1 has a chemical purity of greater than about 95%, by weight, as determined by HPLC analysis. In embodiments, the hydrochloride salt of Compound 1 has a chemical purity of greater than about 99.5%, by weight, as determined by HPLC analysis.

In embodiments, the present disclosure provides a pyruvate salt of Compound 1. In embodiments, the pyruvate salt of Compound 1 is crystalline. In embodiments, the pyruvate salt of Compound 1 is characterized by an XRPD pattern having peaks at 9.4±0.2, 18.8±0.2, and 22.0±0.2 °2θ. In embodiments, the pyruvate salt of Compound 1 is further characterized by peaks in an XRPD pattern at 7.7±0.2, 13.9±0.2, 19.4±0.2, 21.1±0.2, and 22.7±0.2 °2θ. In embodiments, the pyruvate salt of Compound 1 is characterized by an XRPD pattern having peaks at 7.7±0.2, 8.0 0.2, 8.5±0.2, 9.4±0.2, 11.0±0.2, 11.3±0.2, 12.0±0.2, 12.9±0.2, 13.9±0.2, 14.9±0.2, 15.5±0.2, 15.9±0.2, 16.9±0.2, 17.2±0.2, 17.6±0.2, 18.4±0.2, 18.8±0.2, 19.4±0.2, 20.1±0.2, 20.6±0.2, 21.1±0.2, 21.5±0.2, 22.0±0.2, 22.7±0.2, 23.2±0.2, 23.6±0.2, 24.1±0.2, 24.3±0.2, 25.0±0.2, 25.6±0.2, 25.9±0.2, 26.4±0.2, 26.8±0.2, 28.0±0.2, 28.3±0.2, 28.8±0.2, 29.3±0.2, 29.7±0.2, 30.2±0.2, 30.7±0.2, 32.3±0.2, 32.6±0.2, 32.9±0.2, 33.5±0.2, 34.2±0.2, 34.6±0.2, 35.0±0.2, 35.5±0.2, 36.5±0.2, 37.0±0.2, 37.4±0.2, 39.0±0.2, and 39.7±0.2 °2θ. In embodiments, the pyruvate salt of Compound 1 is characterized by an XRPD pattern substantially similar to FIG. 9.

In embodiments, the pyruvate salt of Compound 1 has a chemical purity of greater than about 90%, by weight, as determined by HPLC analysis. In embodiments, the pyruvate salt of Compound 1 has a chemical purity of greater than about 95%, by weight, as determined by HPLC analysis. In embodiments, the pyruvate salt of Compound 1 has a chemical purity of greater than about 99.5%, by weight, as determined by HPLC analysis.

In embodiments, the present disclosure provides a 3-oxodipropanoate salt of Compound 1. In embodiments, the 3-oxodipropanoate salt of Compound 1 is crystalline. In embodiments, the 3-oxodipropanoate salt of Compound 1 is characterized by an XRPD pattern having peaks at 18.0±0.2, 21.7±0.2, and 23.2±0.2 °2θ. In embodiments, the 3-oxodipropanoate salt of Compound 1 is further characterized by peaks in an XRPD pattern at 9.3±0.2, 10.8±0.2, 11.1±0.2, 16.4±0.2, and 19.3±0.2 °2θ. In embodiments, the 3-oxodipropanoate salt of Compound 1 is characterized by an XRPD pattern having peaks at 7.2±0.2, 7.7±0.2, 9.3±0.2, 10.8±0.2, 11.1±0.2, 13.0±0.2, 14.0±0.2, 14.6±0.2, 14.8±0.2, 15.4±0.2, 16.4±0.2, 16.6±0.2, 16.7±0.2, 18.0±0.2, 18.5±0.2, 18.7±0.2, 19.3±0.2, 20.0±0.2, 21.0±0.2, 21.1±0.2, 21.4±0.2, 21.7±0.2, 22.0±0.2, 22.5±0.2, 23.2±0.2, 23.5±0.2, 23.7±0.2, 24.7±0.2, 24.8±0.2, 25.8±0.2, 26.2±0.2, 28.1±0.2, 28.4±0.2, 29.8±0.2, 30.3±0.2, 30.5±0.2, 32.6±0.2, 33.1±0.2, 33.4±0.2, 33.7±0.2, 34.4±0.2, and 37.9±0.2 °2θ. In embodiments, the 3-oxodipropanoate salt of Compound 1 is characterized by an XRPD pattern substantially similar to FIG. 10A.

In embodiments, the 3-oxodipropanoate salt of Compound 1 is crystalline. In embodiments, the 3-oxodipropanoate salt of Compound 1 is characterized by an XRPD pattern having peaks at 19.3±0.2, 21.8±0.2, and 22.3±0.2 °2θ. In embodiments, the 3-oxodipropanoate salt of Compound 1 is further characterized by peaks in an XRPD pattern at 7.5±0.2, 7.8±0.2, 13.8±0.2, 14.9±0.2, and 16.1±0.2 °2θ. In embodiments, the 3-oxodipropanoate salt of Compound 1 is characterized by an XRPD pattern having peaks at 7.5±0.2, 7.8±0.2, 8.3±0.2, 9.7±0.2, 10.5±0.2, 11.8±0.2, 12.4±0.2, 13.0±0.2, 13.5±0.2, 13.8±0.2, 14.9±0.2, 16.1±0.2, 16.5±0.2, 17.4±0.2, 17.9±0.2, 18.9±0.2, 19.3±0.2, 20.1±0.2, 20.6±0.2, 21.8±0.2, 22.3±0.2, 23.0±0.2, 23.4±0.2, 24.3±0.2, 24.8±0.2, 25.8±0.2, 26.3±0.2, 27.0±0.2, 27.7±0.2, 28.5±0.2, 29.0±0.2, 29.4±0.2, 30.3±0.2, 32.3±0.2, 34.2±0.2, 34.8±0.2, and 39.4±0.2 °2θ. In embodiments, the 3-oxodipropanoate salt of Compound 1 is characterized by an XRPD pattern substantially similar to FIG. 10B.

In embodiments, the 3-oxodipropanoate salt of Compound 1 has a chemical purity of greater than about 90%, by weight, as determined by HPLC analysis. In embodiments, the 3-oxodipropanoate salt of Compound 1 has a chemical purity of greater than about 95%, by weight, as determined by HPLC analysis. In embodiments, the 3-oxodipropanoate salt of Compound 1 has a chemical purity of greater than about 99.5%, by weight, as determined by HPLC analysis.

In embodiments, the present disclosure provides an adipate salt of Compound 1. In embodiments, the adipate salt of Compound 1 is crystalline. In embodiments, the adipate salt of Compound 1 is characterized by an XRPD pattern having peaks at 7.5±0.2, 18.0±0.2, and 18.5±0.2 °2θ. In embodiments, the adipate salt of Compound 1 is further characterized by peaks in an XRPD pattern at 9.3±0.2, 14.2±0.2, 15.3±0.2, 21.2±0.2, and 22.3±0.2 °2θ. In embodiments, the adipate salt of Compound 1 is characterized by an XRPD pattern having peaks at 7.5±0.2, 8.6±0.2, 9.3±0.2, 11.3±0.2, 12.6±0.2, 14.2±0.2, 15.0±0.2, 15.3±0.2, 16.3±0.2, 16.7±0.2, 17.1±0.2, 18.0±0.2, 18.5±0.2, 19.3±0.2, 20.8±0.2, 21.2±0.2, 21.7±0.2, 22.3±0.2, 22.8±0.2, 23.4±0.2, 23.8±0.2, 23.9±0.2, 24.3±0.2, 25.1±0.2, 25.8±0.2, 27.0±0.2, 28.1±0.2, 28.6±0.2, 29.8±0.2, 30.8±0.2, 33.7±0.2, 34.8±0.2, 35.5±0.2, 37.4±0.2, and 40.1±0.2 °2θ. In embodiments, the adipate salt of Compound 1 is characterized by an XRPD pattern substantially similar to FIG. 11A.

In embodiments, the adipate salt of Compound 1 is crystalline. In embodiments, the adipate salt of Compound 1 is characterized by an XRPD pattern having peaks at 7.4±0.2, 9.0±0.2, and 14.2±0.2 °2θ. In embodiments, the adipate salt of Compound 1 is further characterized by peaks in an XRPD pattern at 15.1±0.2, 18.0±0.2, 19.7±0.2, 19.9±0.2, and 23.3±0.2 °2θ. In embodiments, the adipate salt of Compound 1 is characterized by an XRPD pattern having peaks at 7.4±0.2, 9.0±0.2, 9.3±0.2, 9.8±0.2, 11.0±0.2, 11.2±0.2, 12.1±0.2, 12.5±0.2, 13.1±0.2, 13.4±0.2, 14.2±0.2, 14.6±0.2, 15.1±0.2, 15.8±0.2, 16.8±0.2, 17.3±0.2, 17.6±0.2, 18.0±0.2, 18.5±0.2, 18.8±0.2, 19.1±0.2, 19.3±0.2, 19.7±0.2, 19.9±0.2, 20.4±0.2, 20.7±0.2, 21.2±0.2, 21.5±0.2, 22.0±0.2, 22.3±0.2, 22.8±0.2, 23.3±0.2, 23.6±0.2, 24.2±0.2, 24.8±0.2, 25.0±0.2, 26.0±0.2, 26.4±0.2, 27.0±0.2, 27.4±0.2, 28.6±0.2, 29.9±0.2, 31.2±0.2, 32.3±0.2, 32.7±0.2, 33.4±0.2, 33.7±0.2, 37.5±0.2, and 38.6±0.2 °2θ. In embodiments, the adipate salt of Compound 1 is characterized by an XRPD pattern substantially similar to FIG. 11B.

In embodiments, the adipate salt of Compound 1 is crystalline. In embodiments, the adipate salt of Compound 1 is characterized by an XRPD pattern having peaks at 7.5±0.2, 17.6±0.2, and 22.8±0.2 °2θ. In embodiments, the adipate salt of Compound 1 is further characterized by peaks in an XRPD pattern at 12.5±0.2, 16.8±0.2, 17.8±0.2, 20.0±0.2, and 22.0±0.2 °2θ. In embodiments, the adipate salt of Compound 1 is characterized by an XRPD pattern having peaks at 7.5±0.2, 9.3±0.2, 11.0±0.2, 11.2±0.2, 12.1±0.2, 12.5±0.2, 14.2±0.2, 14.6±0.2, 15.2±0.2, 15.8±0.2, 16.8±0.2, 17.6±0.2, 17.8±0.2, 18.0±0.2, 18.6±0.2, 19.2±0.2, 20.0±0.2, 20.4±0.2, 20.7±0.2, 21.2±0.2, 22.0±0.2, 22.3±0.2, 22.8±0.2, 23.2±0.2, 23.7±0.2, 23.9±0.2, 24.4±0.2, 24.9±0.2, 25.3±0.2, 25.6±0.2, 25.9±0.2, 26.8±0.2, 27.4±0.2, 29.0±0.2, 29.8±0.2, 30.9±0.2, 31.4±0.2, 32.4±0.2, 35.5±0.2, 36.1±0.2, 37.5±0.2, and 39.0±0.2 °2θ. In embodiments, the adipate salt of Compound 1 is characterized by an XRPD pattern substantially similar to FIG. 11C.

In embodiments, the adipate salt of Compound 1 has a chemical purity of greater than about 90%, by weight, as determined by HPLC analysis. In embodiments, the adipate salt of Compound 1 has a chemical purity of greater than about 95%, by weight, as determined by HPLC analysis. In embodiments, the adipate salt of Compound 1 has a chemical purity of greater than about 99.5%, by weight, as determined by HPLC analysis.

In embodiments, the present disclosure provides an oxalate salt of Compound 1. In embodiments, the oxalate salt of Compound 1 is crystalline. In embodiments, the oxalate salt of Compound 1 is characterized by an XRPD pattern having peaks at 13.9±0.2, 21.9±0.2, and 24.8±0.2 °2θ. In embodiments, the oxalate salt of Compound 1 is further characterized by peaks in an XRPD pattern at 13.4±0.2, 14.3±0.2, 17.1±0.2, 19.4±0.2, and 27.6±0.2 °2θ. In embodiments, the oxalate salt of Compound 1 is characterized by an XRPD pattern having peaks at 8.9±0.2, 11.6±0.2, 13.4±0.2, 13.9±0.2, 14.3±0.2, 16.3±0.2, 17.1±0.2, 18.1±0.2, 19.4±0.2, 19.3±0.2, 20.2±0.2, 21.5±0.2, 21.9±0.2, 23.1±0.2, 24.1±0.2, 24.8±0.2, 25.0±0.2, 26.7±0.2, 27.6±0.2, 34.4±0.2, and 34.8±0.2 °2θ. In embodiments, the oxalate salt of Compound 1 is characterized by an XRPD pattern substantially similar to FIG. 12.

In embodiments, the oxalate salt of Compound 1 has a chemical purity of greater than about 90%, by weight, as determined by HPLC analysis. In embodiments, the oxalate salt of Compound 1 has a chemical purity of greater than about 95%, by weight, as determined by HPLC analysis. In embodiments, the oxalate salt of Compound 1 has a chemical purity of greater than about 99.5%, by weight, as determined by HPLC analysis.

In embodiments, the present disclosure provides a pharmaceutical composition comprising a pharmaceutically acceptable salt of Compound 1 disclosed herein.

In embodiments, the present disclosure provides a fumarate salt of Compound 1:

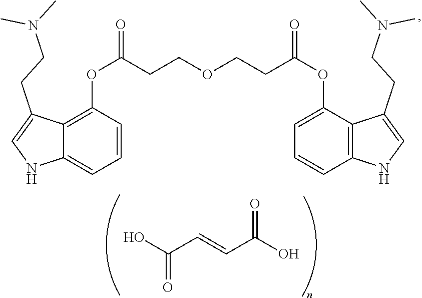

wherein n is from 1 to 3.

In embodiments, the fumarate salt has the following formula:

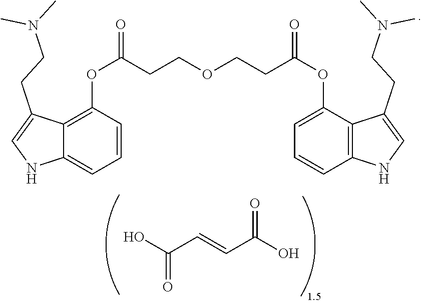

In embodiments, the fumarate salt has the following formula:

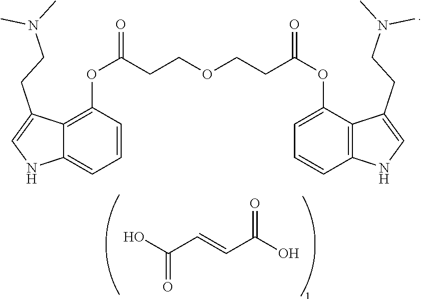

In embodiments, the succinate salt has the following formula:

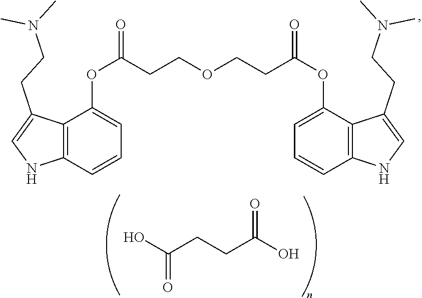

wherein n is from 1 to 2.

In embodiments, the hydrochloride salt has the following formula:

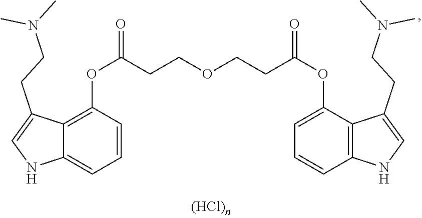

wherein n is from 1 to 2.

In embodiments, the pyruvate salt has the following formula:

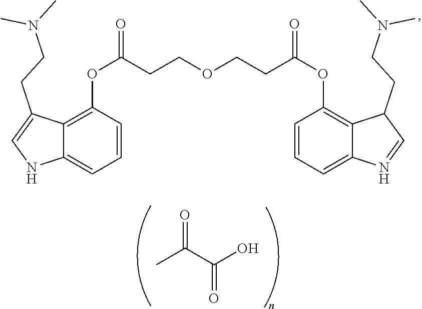

wherein n is from 1 to 2.

In embodiments, the 3-oxodipropanoate salt has the following formula:

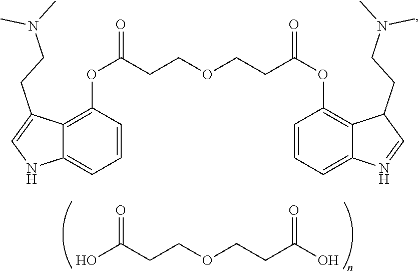

wherein n is from 1 to 2.

In embodiments, the adipate salt has the following formula:

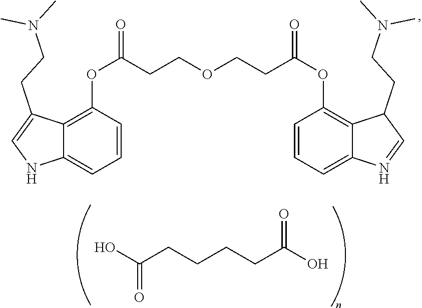

wherein n is from 1 to 3.

In embodiments, the oxalate salt has the following formula:

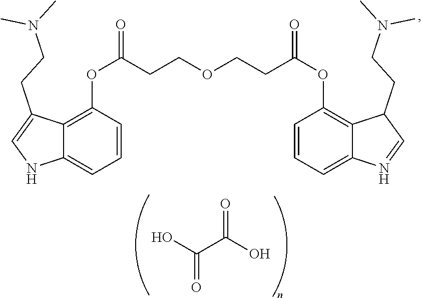

wherein n is from 1 to 2.

In embodiments, the present disclosure provides a compound, having the following structure (A):

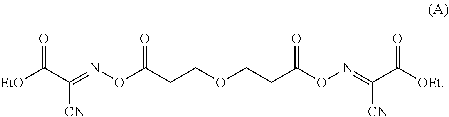 [SMILES: CCOC(=O)/C(C#N)=N/OC(=O)CCOCCC(=O)O/N=C(\C#N)C(=O)OCC]

In embodiments, the present disclosure provides a method of manufacturing the compound of structure (A), comprising:
(i) reacting 3,3′-oxydipropionic acid with thionyl dichloride to form 3,3′-oxydipropionyl dichloride; and
(ii) reacting 3,3′-oxydipropionyl dichloride with ethyl 2-cyano-2-(hydroxyimino)acetate (bis-oxyma) in the presence of a base and a solvent to form the compound of structure (A).

In embodiments, the present disclosure provides a method of manufacturing the pharmaceutically acceptable salt of compound 1 described herein, comprising:
(i) reacting the compound of structure (A) with psilocin in the presence of a base and a solvent to form free base Compound 1; and
(ii) reacting the free base Compound 1 with an acid to form the pharmaceutically acceptable salt.

### BRIEF DESCRIPTION OF THE DRAWINGS

FIG. 1 shows ¹H NMR characterization of the free base of Compound 1.

FIGS. 2A-2B show characterization of the bis-formate salt of Compound 1: XRPD (FIG. 2A) and DSC (FIG. 2B).

FIGS. 3A-3F show characterization of the fumarate (1.0 equivalents) of Compound 1: ¹HMR (FIG. 3A), XRPD (FIG. 3B), DSC (FIG. 3C), TGA (FIG. 3D), GVS (FIG. 3E), and XRPD pre and post GVS (FIG. 3F) for Form 1.

FIGS. 3G-3O show characterization of the various polymorphic forms of the fumarate (1.0 equivalents) of Compound 1: XRPD overlay of the forms (FIG. 3G), XRPD of Form 2 (FIG. 3H), XRPD of Form 3 (FIG. 3I), XRPD of Form 4 (FIG. 3J), XRPD of Form 5 (FIG. 3K), XRPD of Form 6 (FIG. 3L), XRPD of Form 7 (FIG. 3M), XRPD of Form 8 (FIG. 3N), and XRPD of Form 9 (FIG. 3O).

FIGS. 4A-4I show characterization of the sesquifumarate (1.5 equivalents) salt of Compound 1: XRPD (FIG. 4A), ¹H NMR (FIG. 4B), DSC (FIG. 4C), TGA (FIG. 4D), GVS (FIG. 4E), XRPD pre and post GVS (FIG. 4F), XRPD before and after freezer storage for 17 weeks (FIG. 4G), XRPD before and after freezer storage under stress conditions for 12 weeks (FIG. 4H), and XRPD overlay from polymorph assessment (FIG. 4I).

FIGS. 4J-4L show single crystal structures of the sesquifumarate salt (1.5 equivalents) salt of Compound 1: asymmetric crystal unit cell with atom labeling and thermal ellipsoids drawn at 50% probability (FIG. 4J) and two unit cells with stick model representation (FIG. 4K-4L).

FIGS. 5A-5C show characterization of the succinate salt of Compound 1: XRPD (FIG. 5A), DSC (FIG. 5B), and TGA (FIG. 5C)

FIGS. 6A-6B show characterization of the L-tartrate salt of Compound 1: XRPD (FIG. 6A) and DSC (FIG. 6B).

FIG. 7 shows XRPD characterization of the succinate (1.5 equivalents) salt of Compound 1.

FIGS. 8A-8B show characterization of the various polymorphic forms of the hydrochloride salt of Compound 1: XRPD of Form 10 (FIG. 8A) and XRPD of Form 11 (FIG. 8B).

FIG. 9 shows XRPD characterization of the pyruvate salt of Compound 1.

FIGS. 10A-10B show characterization of the various polymorphic forms of the 3-oxodipropanoate salt of Compound 1: XRPD of Form 12 (FIG. 10A) and XRPD of Form 13 (FIG. 10B).

FIGS. 11A-11C show characterization of the various polymorphic forms of the adipate salt of Compound 1: XRPD of Form 14 (FIG. 11A), XRPD of Form 15 (FIG. 11B), and XRPD of Form 16 (FIG. 11C).

FIG. 12 shows XRPD characterization of the oxalate salt of Compound 1.

### DETAILED DESCRIPTION

The present disclosure relates to salt forms of Compound 1 to include crystalline forms. Multiple salt forms of Compound 1 were produced. The sesquifumarate salt of Compound 1 exhibited a high degree of crystallinity and stability relative to other salt forms (e.g., monofumarate, bis-formate, succinate, and L-tartrate salt forms of Compound 1) and is suitable for large scale manufacturing, indicating that the sesquifumarate salt of Compound 1 has the desired characteristics for further pharmaceutical development.

### Definitions

While the following terms are believed to be well understood by one of ordinary skill in the art, the following definitions are set forth to facilitate explanation of the presently disclosed subject matter.

Compound 1 is 3-(2-(dimethylamino)ethyl)-1H-inden-4-yl 3-(3-((1-(2-(dimethylamino)ethyl)-1H-inden-7-yl)oxy)-3-oxopropoxy)propanoate having the structure below.

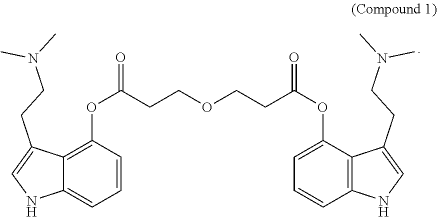

The term “substantially similar” as used herein with regards to an analytical spectrum, such as an XRPD pattern, means that a spectrum resembles the reference spectrum in peak locations, allowing for variability appropriate in the art. For example, two spectra may be regarded as “substantially similar” when the two spectra share defining characteristics sufficient to differentiate them from a spectrum obtained for a different solid form. Relative peak intensities may vary depending on instrumentation and conditions, and such variations are taken into account when determining if two spectra are substantially similar. In embodiments, spectra or characterization data that are substantially similar to those of a reference crystalline form are understood by those of ordinary skill in the art to correspond to the same crystalline form as the particular reference. In analyzing whether spectra or characterization data are substantially similar, a person of ordinary skill in the art understands that particular characterization data points may vary to a reasonable extent while still describing a given solid form, due to, for example, experimental error and routine sample-to-sample analysis.

All XRPD peaks and patterns are given in °2θ using Cu Kα1 radiation at a wavelength of 1.5406 Å. The values of degree 2θ allow appropriate error margins. For example, the degree 2θ of about “17.5±0.2” denotes a range from about 17.3 to 17.7 degree 28. Depending on the sample preparation techniques, the calibration techniques applied to the instruments, human operational variation, and etc., those skilled in the art recognize that the appropriate error of margins for a XRPD can be 0.2, which includes any value below ±0.2 such as ±0.1; ±0.05; or less.

TGA and DSC thermograms for a given form of the same compound will vary within a margin of error. The values of a single peak, expressed in degree Celsius, allow appropriate error margins. Typically, the error margins are represented by “±”. For example, the single peak characteristic value of about “120±5” denotes a range from about 115 to 125. Depending on the sample preparation techniques, the calibration techniques applied to the instruments, human operational variations etc., those skilled in the art recognize that the appropriate error of margins for a single peak characteristic value can be 5, which includes any value below ±5 such as 4, 3.5, ±3, 2.5; ±2.0; 1.5; 1.0; ±0.5; or less.

### Salt Forms of Compound 1

Aspects of the present disclosure relate to salt forms of Compound 1.

In embodiments, the present disclosure provides a fumarate salt of Compound 1, succinate salt of Compound 1, L-tartrate salt of Compound 1, hydrochloride salt of Compound 1, pyruvate salt of Compound 1, 3-oxodipropanoate salt of Compound 1, adipate salt of Compound 1, or oxalate salt of Compound 1. In embodiments, the fumarate salt is a monofumarate salt, sesquifumarate salt, or a bis-formate salt of Compound 1.

In embodiments, the present disclosure provides a monofumarate salt of Compound 1. In embodiments the monofumarate salt of Compound 1 has the following formula:

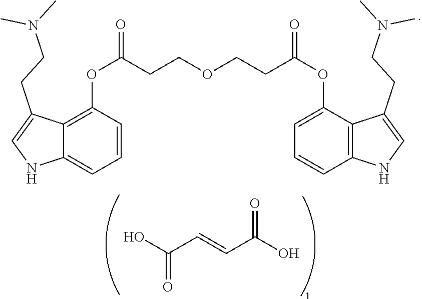

In embodiments, monofumarate salt of Compound 1 is crystalline.

In embodiments, the monofumarate salt of Compound 1 is crystalline Form 1, Form 2, Form 3, Form 4, Form 5, Form 6, Form 7, Form 8, Form 9, or mixtures thereof.

### Form 1:

In embodiments, the monofumarate salt of Compound 1, is characterized by an XRPD pattern having peaks at 12.8±0.2, 18.7±0.2, and 23.2±0.2 °2θ. In embodiments, the variance at any of these peaks is ±0.1 °2θ.

In embodiments, the monofumarate salt of Compound 1 is characterized by an XRPD having peaks at 12.8±0.2, 18.7±0.2, and 23.2±0.2 °2θ and at least one (e.g., 1, 2, 3, 4, or 5) peak in an XRPD pattern selected from 7.2±0.2, 10.4±0.2, 17.9±0.2, 19.3±0.2, and 21.4±0.2 °2θ. In embodiments, the variance at any of these peaks is ±0.1 °2θ.

In embodiments, the monofumarate salt of Compound 1 is characterized by an XRPD having peaks at 7.2±0.2, 10.4±0.2, 12.8±0.2, 18.7±0.2, 17.9±0.2, 19.3±0.2, 21.4±0.2, and 23.2±0.2, °2θ. In embodiments, the variance at any of these peaks is ±0.1 °2θ.

In embodiments, the monofumarate salt of Compound 1 is characterized by an XRPD pattern having one or more peaks at 5.3±0.2, 7.2±0.2, 7.7±0.2, 8.9±0.2, 10.4±0.2, 11.3±0.2, 12.8±0.2, 13.7±0.2, 14.4±0.2, 14.7±0.2, 15.4±0.2, 16.5±0.2, 16.9±0.2, 17.3±0.2, 17.9±0.2, 18.7±0.2, 19.3±0.2, 19.8±0.2, 20.8±0.2, 21.0±0.2, 21.4±0.2, 21.8±0.2, 22.8±0.2, 23.2±0.2, 24.5±0.2, 24.8±0.2, 25.7±0.2, 26.4±0.2, 27.4±0.2, 28.6±0.2, 29.0±0.2, 29.5±0.2, 30.2±0.2, 30.6±0.2, 31.3±0.2, 32.3±0.2, and 32.7±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

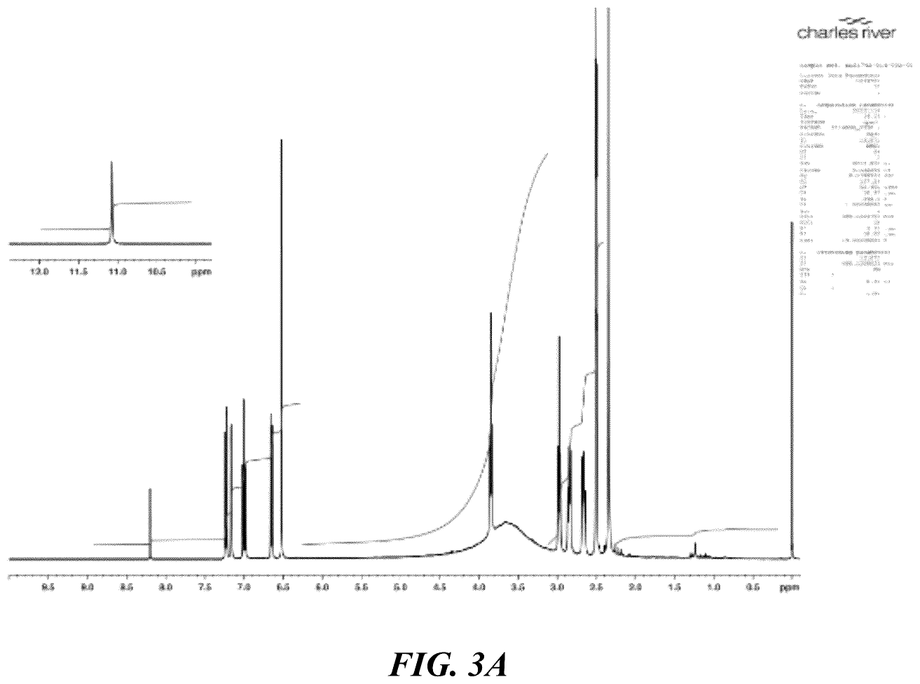

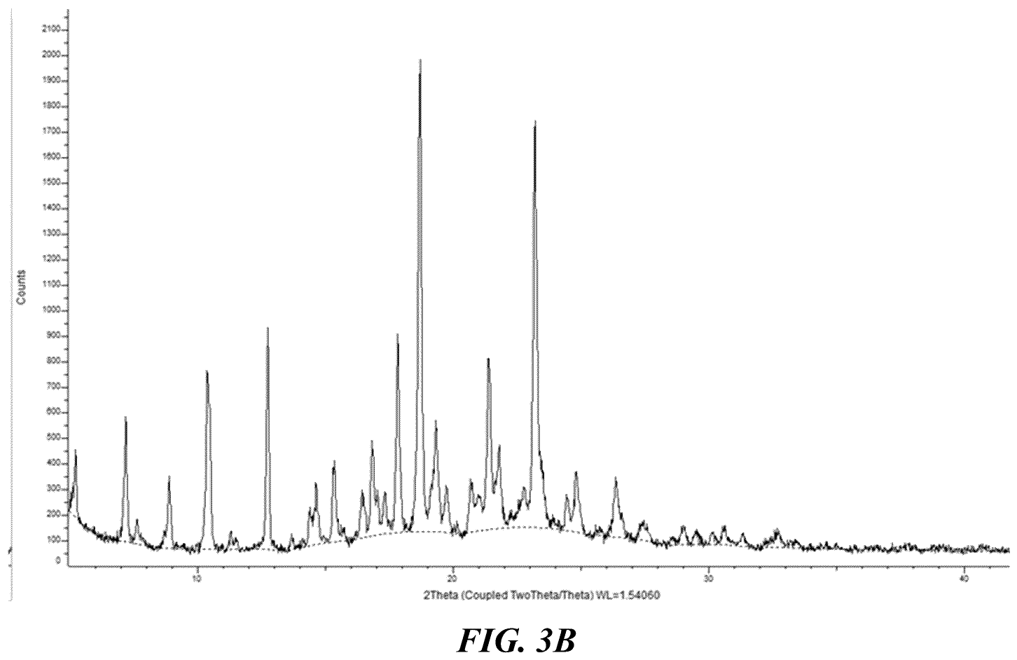

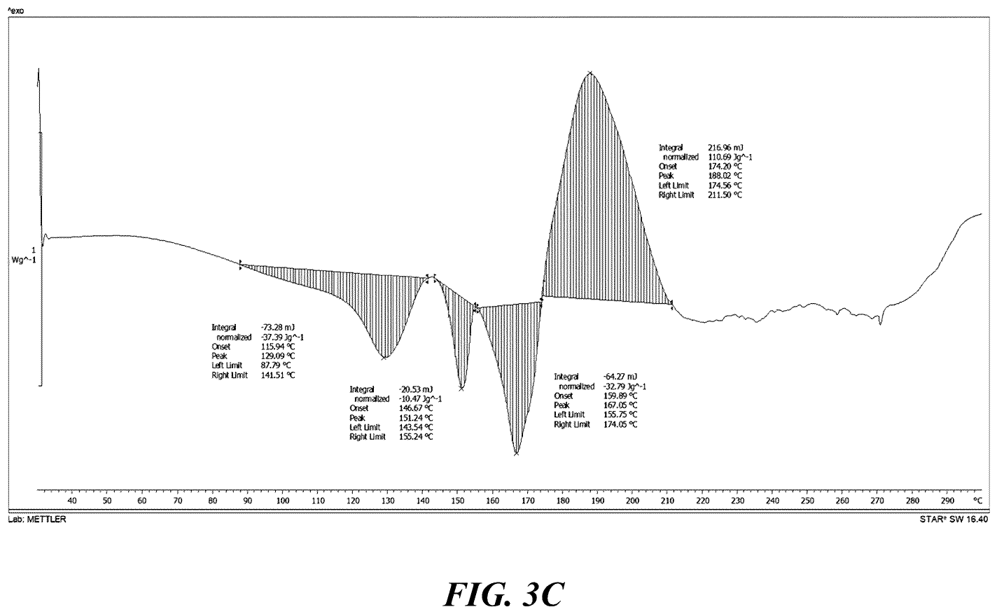

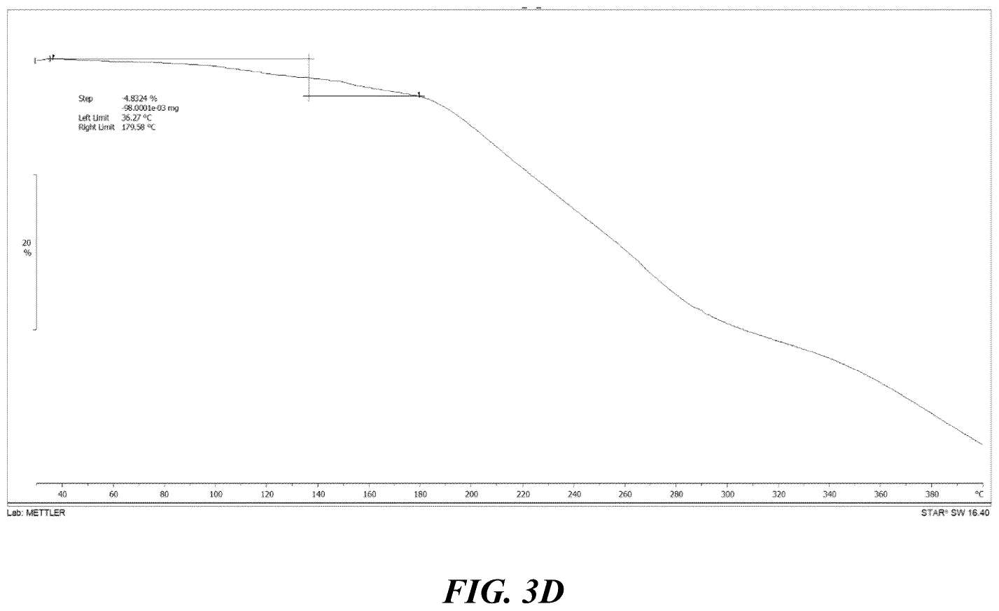

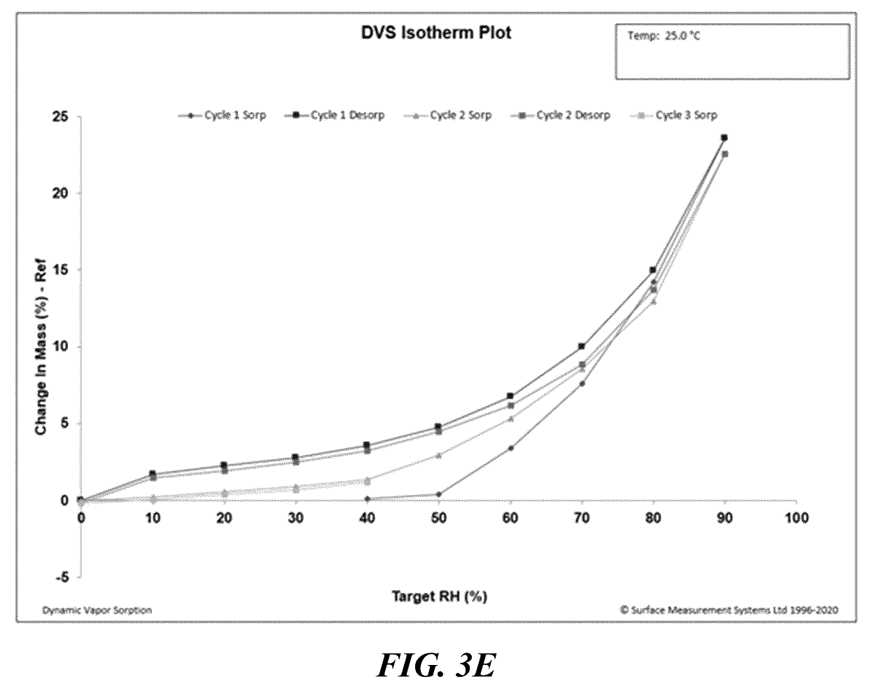

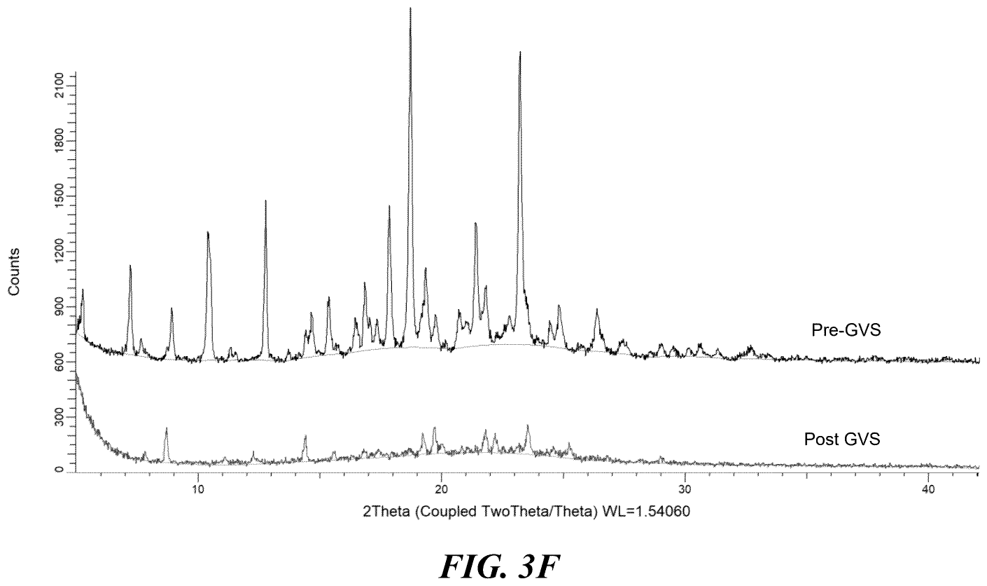

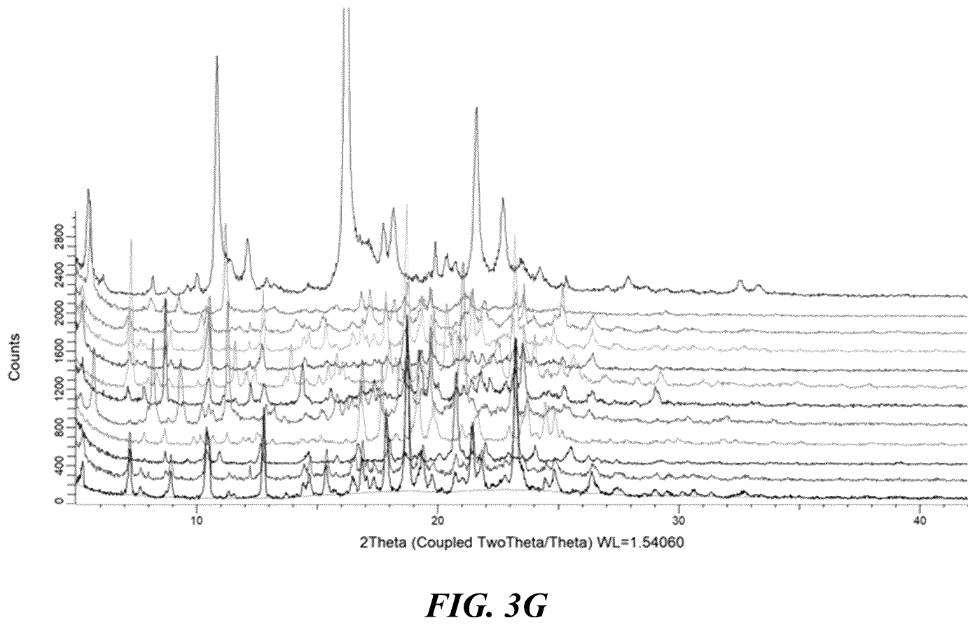

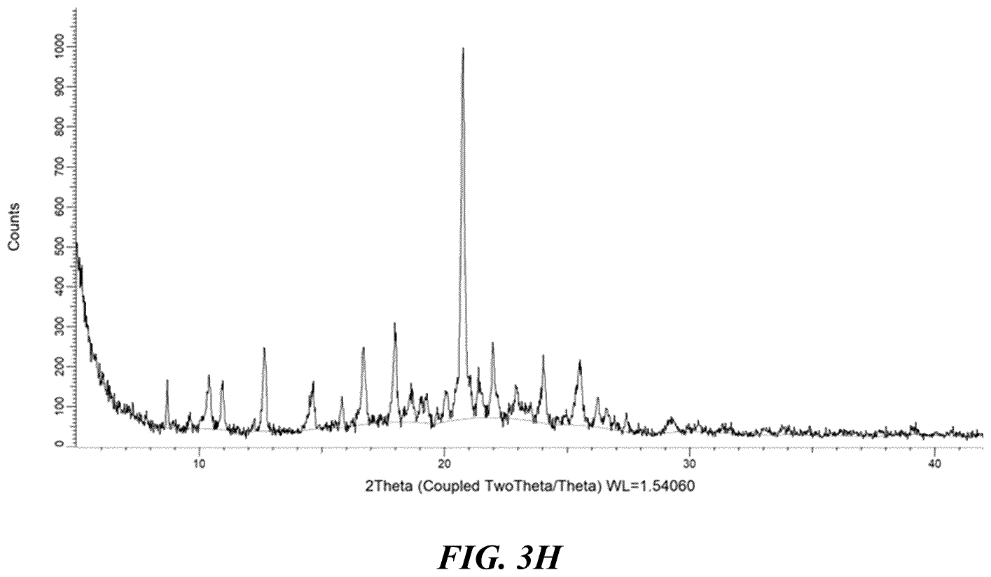

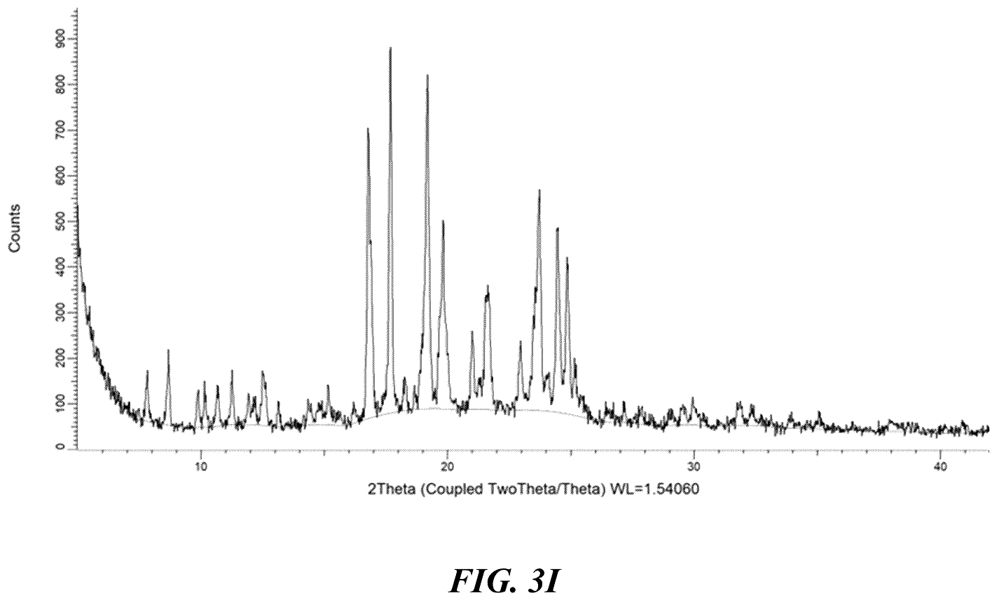

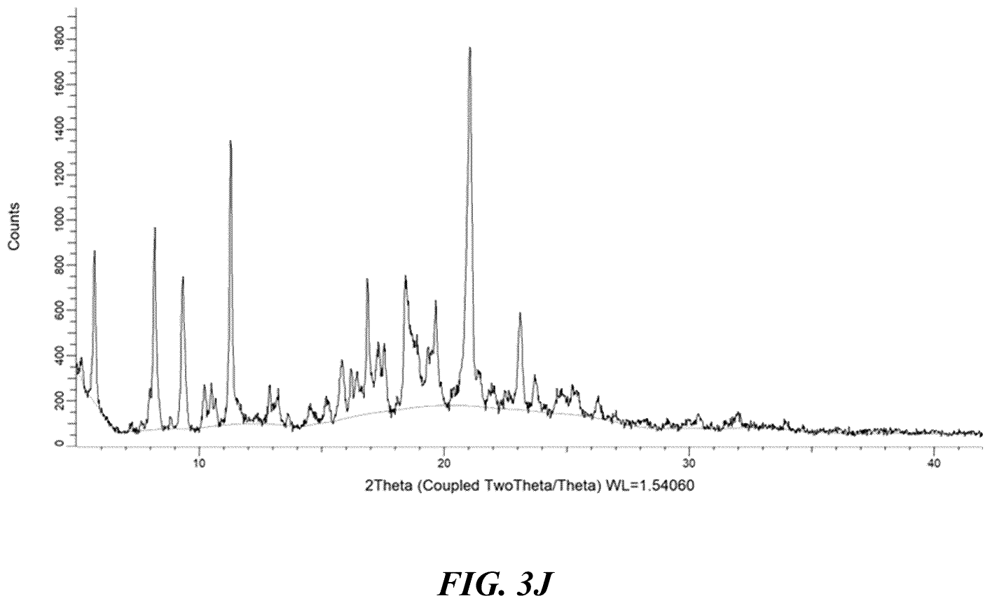

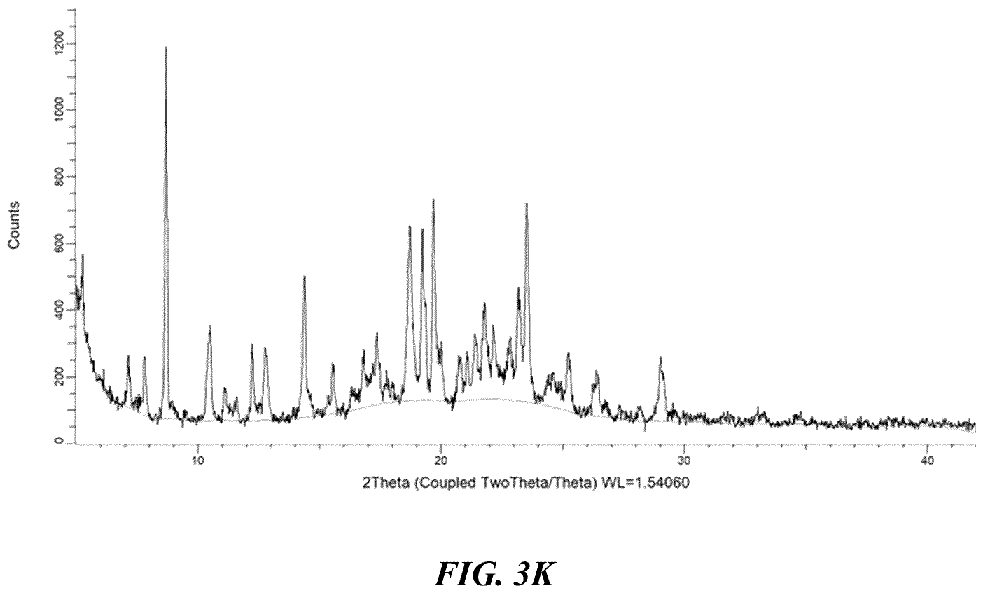

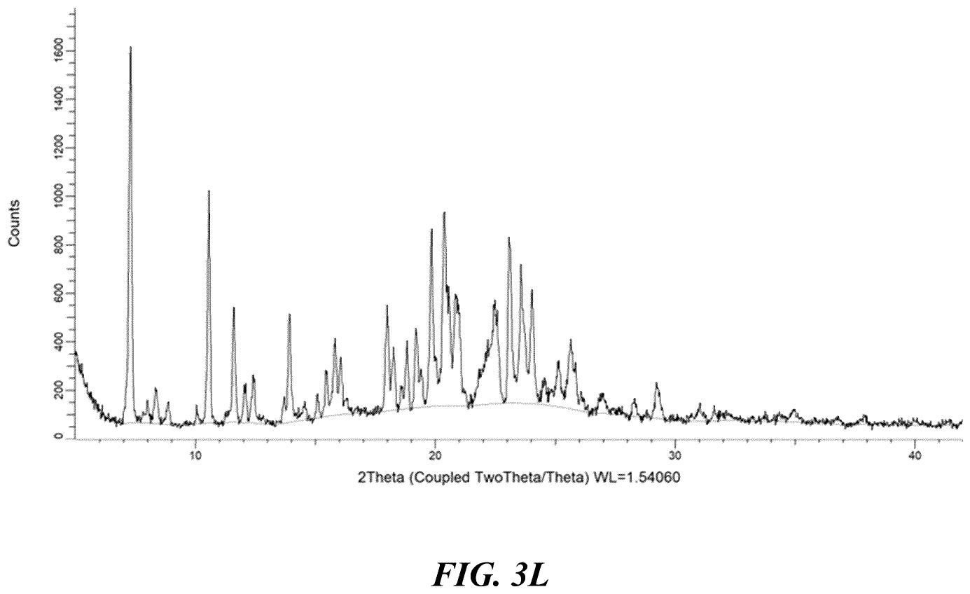

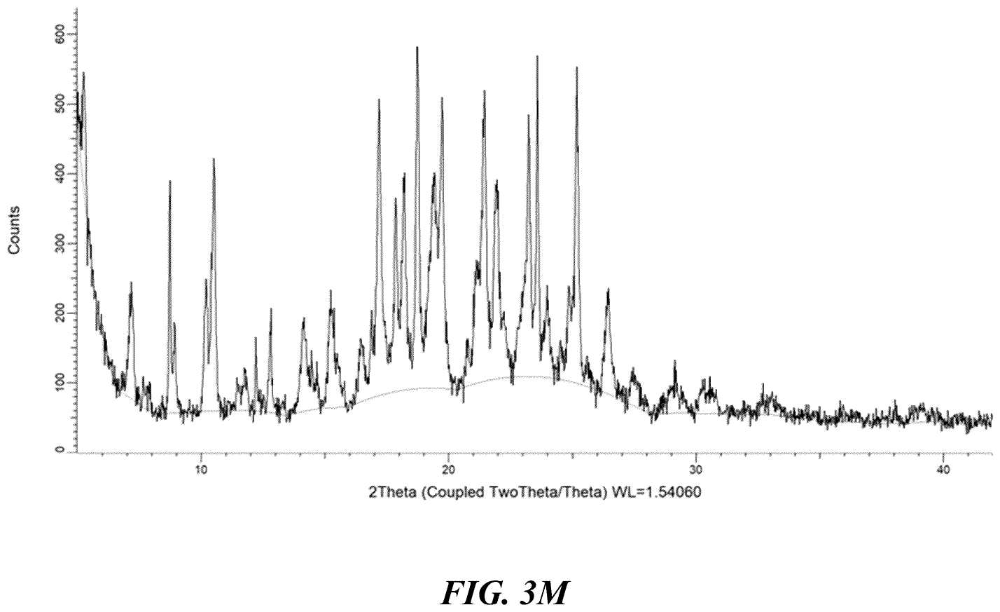

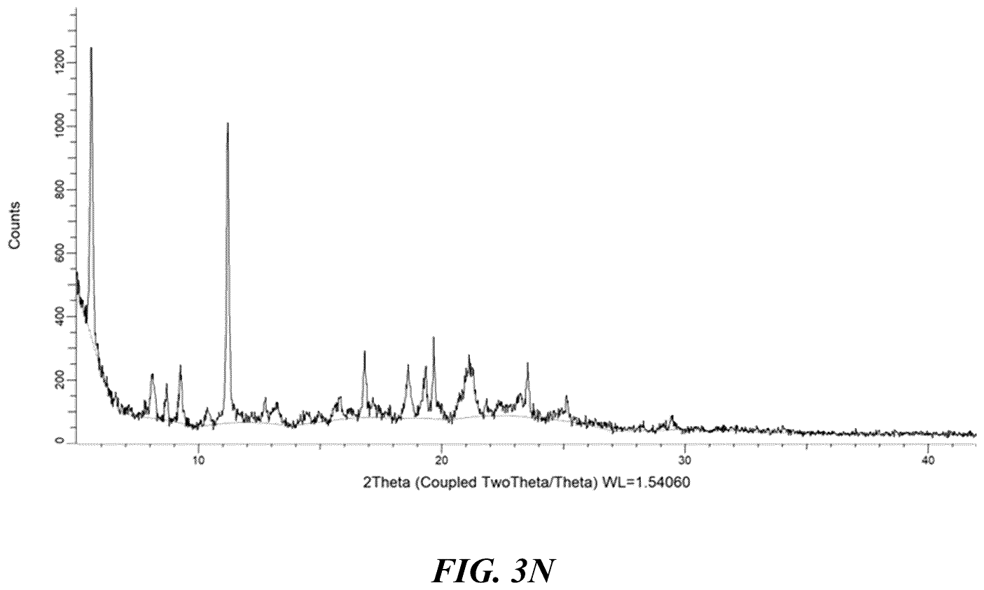

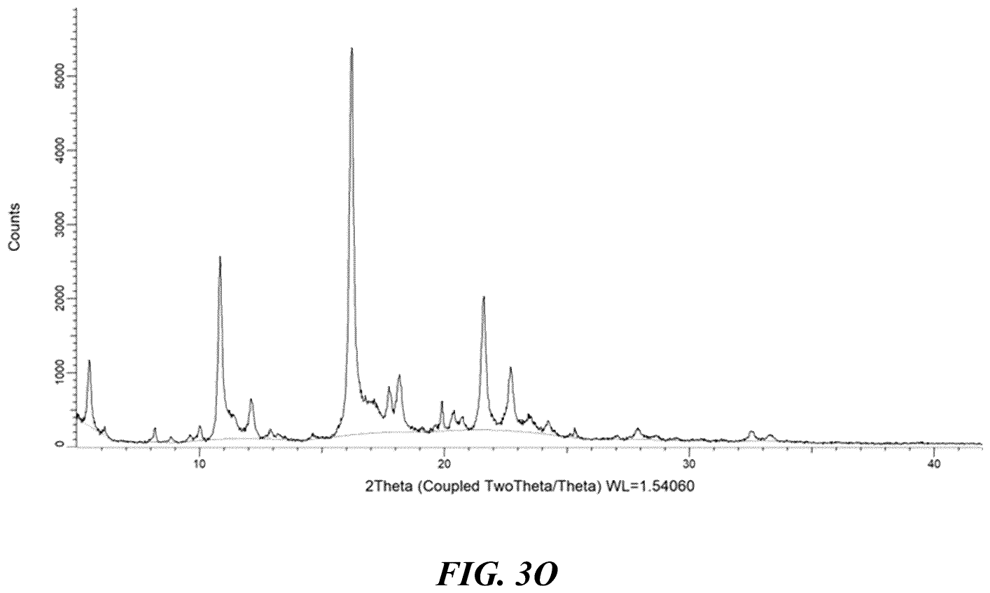

In embodiments, the monofumarate salt of Compound 1 is characterized by an XRPD pattern substantially similar to FIG. 3B.

In embodiments, monofumarate salt of Compound 1 is characterized by an XRPD pattern comprising peaks shown in Table A.

TABLE A
XRPD Peak Positions for the Monofumarate Salt of Compound 1

| Position °2θ | Relative Intensity |
|---|---|
| 5.3 | 11.34% |
| 7.2 | 24.13% |
| 7.7 | 4.88% |
| 8.9 | 15.20% |
| 10.4 | 33.56% |
| 11.3 | 3.44% |
| 12.8 | 45.24% |
| 13.7 | 2.41% |
| 14.4 | 7.27% |
| 14.7 | 10.87% |
| 15.4 | 15.15% |
| 16.5 | 8.66% |
| 16.9 | 19.21% |
| 17.3 | 8.17% |
| 17.9 | 40.66% |
| 18.7 | 100.00% |
| 19.3 | 24.48% |
| 19.8 | 9.10% |
| 20.8 | 9.06% |
| 21.0 | 6.36% |
| 21.4 | 35.02% |
| 21.8 | 15.97% |
| 22.8 | 8.61% |
| 23.2 | 84.94% |
| 24.5 | 6.25% |
| 24.8 | 13.77% |
| 25.7 | 2.20% |
| 26.4 | 12.23% |
| 27.4 | 3.71% |
| 28.6 | 1.57% |
| 29.0 | 3.41% |
| 29.5 | 2.22% |
| 30.2 | 1.93% |
| 30.6 | 2.15% |
| 31.3 | 3.14% |
| 32.3 | 1.15% |
| 32.7 | 4.60% |

In embodiments, monofumarate salt of Compound 1 is characterized by one, two, three, four, five, six, seven, eight, nine, ten, eleven, or more, XRPD peaks listed in Table A.

In embodiments, the monofumarate salt of Compound 1 is characterized by a DSC thermogram comprising an endothermic event between 88±5° C. to 142±5° C., 144±5° C. to 155±5° C., and 156±5° C. to 174±5° C.

In embodiments, the monofumarate salt of Compound 1 is characterized by a DSC comprising an exothermic event between 175±5° C. to 212±5° C.

In embodiments, the monofumarate salt of Compound 1 is characterized by a DSC thermogram substantially similar to FIG. 3C.

In embodiments the monofumarate salt of Compound 1 is characterized by about a 4.8% weight loss from 36±5° C. to 180±5° C. as determined by TGA.

In embodiments, the monofumarate salt of Compound 1 is characterized by a thermogravimetric thermogram substantially similar to FIG. 3D.

In embodiments, the monofumarate salt of Compound 1 of Form I has a chemical purity of greater than about 80%, about 85%, about 90%, about 95%, about 96%, about 97%, about 98%, about 99%, 99.5%, or about 100% by weight as determined by HPLC.

### Form 2:

In embodiments, the monofumarate salt of Compound 1, is characterized by an XRPD pattern having peaks at 12.7±0.2, 18.0±0.2, and 20.7±0.2 °2θ. In embodiments, the variance at any of these peaks is ±0.1 °2θ.

In embodiments, the monofumarate salt of Compound 1 is characterized by an XRPD having peaks at 12.7±0.2, 18.00 0.2, and 20.7±0.2 °2θ and at least one (e.g., 1, 2, 3, 4, or 5) peak in an XRPD pattern selected from 10.4±0.2, 16.7±0.2, 22.0 0.2, 24.0±0.2, and 25.5±0.2 °2θ. In embodiments, the variance at any of these peaks is ±0.1 °2θ.

In embodiments, the monofumarate salt of Compound 1 is characterized by an XRPD having peaks at 10.4±0.2, 12.7±0.2, 16.7±0.2, 18.0±0.2, 20.7±0.2, 22.0±0.2, 24.0±0.2, and 25.5±0.2 °2θ. In embodiments, the variance at any of these peaks is ±0.1 °2θ.

In embodiments, the monofumarate salt of Compound 1 is characterized by an XRPD pattern having one or more peaks at 8.7±0.2, 9.6±0.2, 10.4±0.2, 10.9±0.2, 12.7±0.2, 14.6±0.2, 15.8±0.2, 16.7±0.2, 18.0±0.2, 18.6±0.2, 19.1±0.2, 19.3±0.2, 19.7±0.2, 20.1±0.2, 20.7±0.2, 21.4±0.2, 22.0±0.2, 22.9±0.2, 23.4±0.2, 24.0±0.2, 25.5±0.2, 26.3±0.2, 26.6±0.2, 27.4±0.2, 29.3±0.2, 30.3±0.2, 33.8±0.2, 34.9±0.2, and 37.7±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3H.

In embodiments, monofumarate salt of Compound 1 is characterized by an XRPD pattern comprising peaks shown in Table B.

TABLE B
XRPD Peak Positions for the Monofumarate
Salt of Compound 1 (“Form 2”)

| Peak Position °2θ | Relative Intensity |
|---|---|
| 8.7 | 12.85% |
| 9.6 | 4.13% |
| 10.4 | 13.58% |
| 10.9 | 9.76% |
| 12.7 | 20.97% |
| 14.6 | 11.27% |
| 15.8 | 8.87% |
| 16.7 | 19.62% |
| 18.0 | 21.19% |
| 18.6 | 11.97% |
| 19.1 | 4.43% |
| 19.3 | 7.50% |
| 19.7 | 3.27% |
| 20.1 | 6.86% |
| 20.7 | 100.00% |
| 21.4 | 4.40% |
| 22.0 | 20.49% |
| 22.9 | 9.62% |
| 23.4 | 3.73% |
| 24.0 | 20.02% |
| 25.5 | 15.93% |
| 26.3 | 8.18% |
| 26.6 | 5.83% |
| 27.4 | 6.40% |
| 29.3 | 2.53% |
| 30.3 | 3.87% |
| 33.8 | 2.28% |
| 34.9 | 3.20% |
| 37.7 | 2.30% |

In embodiments, monofumarate salt of Compound 1 is characterized by one, two, three, four, five, six, seven, eight, nine, ten, eleven, or more, XRPD peaks listed in Table B.

In embodiments, the monofumarate salt of Compound 1 of Form 2 has a chemical purity of greater than about 80%, about 85%, about 90%, about 95%, about 96%, about 97%, about 98%, about 99%, 99.5%, or about 100% by weight as determined by HPLC.

### Form 3:

In embodiments, the monofumarate salt of Compound 1, is characterized by an XRPD pattern having peaks at 16.8±0.2, 17.7±0.2, and 19.2±0.2 °2θ. In embodiments, the variance at any of these peaks is ±0.1 °2θ.

In embodiments, the monofumarate salt of Compound 1 is characterized by an XRPD having peaks at 16.8±0.2, 17.7±0.2, and 19.2±0.2 °2θ and at least one (e.g., 1, 2, 3, 4, or 5) peak in an XRPD pattern selected from 19.8±0.2, 21.6±0.2, 23.7±0.2, 24.5±0.2, and 24.9±0.2 °2θ. In embodiments, the variance at any of these peaks is ±0.1 °2θ.

In embodiments, the monofumarate salt of Compound 1 is characterized by an XRPD having peaks at 16.8±0.2, 17.7±0.2, 19.2±0.2, 19.8±0.2, 21.6±0.2, 23.7±0.2, 24.5±0.2, and 24.9±0.2 °2θ. In embodiments, the variance at any of these peaks is ±0.1 °2θ.

In embodiments, the monofumarate salt of Compound 1 is characterized by an XRPD pattern having one or more peaks at 7.8±0.2, 8.7±0.2, 9.9±0.2, 10.2±0.2, 10.7±0.2, 11.3±0.2, 11.9±0.2, 12.1±0.2, 12.5±0.2, 13.1±0.2, 14.4±0.2, 15.2±0.2, 16.2±0.2, 16.8±0.2, 17.7±0.2, 18.3±0.2, 19.2±0.2, 19.8±0.2, 21.0±0.2, 21.6±0.2, 23.0±0.2, 23.7±0.2, 24.0±0.2, 24.5±0.2, 24.9±0.2, 25.2±0.2, 26.5±0.2, 27.2±0.2, 27.9±0.2, 29.1±0.2, 29.5±0.2, 29.9±0.2, 31.9±0.2, 32.3±0.2, 34.0±0.2, and 35.1±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3I.

In embodiments, monofumarate salt of Compound 1 is characterized by an XRPD pattern comprising peaks shown in Table C.

TABLE C
XRPD Peak Positions for the Monofumarate
Salt of Compound 1 (“Form 3”)

| Peak Position °2θ | Relative Intensity |
|---|---|
| 7.8 | 12.93% |
| 8.7 | 20.99% |
| 9.9 | 8.79% |
| 10.2 | 11.32% |
| 10.7 | 7.11% |
| 11.3 | 14.18% |
| 11.9 | 7.98% |
| 12.1 | 7.99% |
| 12.5 | 12.44% |
| 13.1 | 5.09% |
| 14.4 | 5.07% |
| 15.2 | 11.39% |
| 16.2 | 5.61% |
| 16.8 | 71.96% |
| 17.7 | 100.00% |
| 18.3 | 9.28% |
| 19.2 | 88.78% |
| 19.8 | 51.48% |
| 21.0 | 20.80% |
| 21.6 | 33.44% |
| 23.0 | 18.27% |
| 23.7 | 58.01% |
| 24.0 | 8.01% |
| 24.5 | 53.85% |
| 24.9 | 38.47% |
| 25.2 | 10.61% |
| 26.5 | 2.46% |
| 27.2 | 5.41% |
| 27.9 | 4.70% |
| 29.1 | 4.37% |
| 29.5 | 4.63% |
| 29.9 | 7.91% |
| 31.9 | 6.67% |
| 32.3 | 3.38% |
| 34.0 | 3.86% |
| 35.1 | 5.43% |

In embodiments, monofumarate salt of Compound 1 is characterized by one, two, three, four, five, six, seven, eight, nine, ten, eleven, or more, XRPD peaks listed in Table C.

In embodiments, the monofumarate salt of Compound 1 of Form 3 has a chemical purity of greater than about 80%, about 85%, about 90%, about 95%, about 96%, about 97%, about 98%, about 99%, 99.5%, or about 100% by weight as determined by HPLC.

### Form 4:

In embodiments, the monofumarate salt of Compound 1, is characterized by an XRPD pattern having peaks at 8.2±0.2, 11.3±0.2, and 21.0±0.2 °2θ. In embodiments, the variance at any of these peaks is ±0.1 °2θ.

In embodiments, the monofumarate salt of Compound 1 is characterized by an XRPD having peaks at 8.2±0.2, 11.3±0.2, and 21.0±0.2 °2θ and at least one (e.g., 1, 2, 3, 4, or 5) peak in an XRPD pattern selected from 5.7±0.2, 9.3±0.2, 16.9±0.2, 18.5±0.2, and 19.7±0.2 °2θ. In embodiments, the variance at any of these peaks is ±0.1 °2θ.

In embodiments, the monofumarate salt of Compound 1 is characterized by an XRPD having peaks at 5.7±0.2, 8.2±0.2, 9.3±0.2, 11.3±0.2, 16.9±0.2, 18.5±0.2, 19.7±0.2, and 21.0±0.2 °2θ. In embodiments, the variance at any of these peaks is ±0.1 °2θ.

In embodiments, the monofumarate salt of Compound 1 is characterized by an XRPD pattern having one or more peaks at 5.7±0.2, 7.2±0.2, 7.6±0.2, 8.2±0.2, 8.8±0.2, 9.3±0.2, 10.2±0.2, 10.5±0.2, 11.3±0.2, 12.3±0.2, 12.9±0.2, 13.2±0.2, 13.6±0.2, 14.5±0.2, 15.2±0.2, 15.8±0.2, 16.2±0.2, 16.4±0.2, 16.9±0.2, 17.3±0.2, 17.6±0.2, 18.5±0.2, 18.9±0.2, 19.4±0.2, 19.7±0.2, 20.4±0.2, 21.0±0.2, 21.4±0.2, 21.9±0.2, 22.6±0.2, 23.1±0.2, 23.7±0.2, 24.8±0.2, 25.3±0.2, 25.4±0.2, 26.3±0.2, 27.0±0.2, 28.2±0.2, 29.1±0.2, 30.4±0.2, 32.0±0.2, and 33.9±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3J.

In embodiments, monofumarate salt of Compound 1 is characterized by an XRPD pattern comprising peaks shown in Table D.

TABLE D
XRPD Peak Positions for the Monofumarate
Salt of Compound 1 (“Form 4”)

| Peak Position °2θ | Relative Intensity |
|---|---|
| 5.7 | 37.45% |
| 7.2 | 2.05% |
| 7.6 | 1.83% |
| 8.2 | 53.95% |
| 8.8 | 3.01% |
| 9.3 | 40.38% |
| 10.2 | 11.58% |
| 10.5 | 12.79% |
| 11.3 | 79.17% |
| 12.3 | 1.75% |
| 12.9 | 10.64% |
| 13.2 | 7.05% |
| 13.6 | 2.79% |
| 14.5 | 3.41% |
| 15.2 | 3.97% |
| 15.8 | 16.37% |
| 16.2 | 12.43% |
| 16.4 | 12.71% |
| 16.9 | 35.13% |
| 17.3 | 18.45% |
| 17.6 | 17.29% |
| 18.5 | 32.84% |
| 18.9 | 20.66% |
| 19.4 | 16.30% |
| 19.7 | 30.11% |
| 20.4 | 5.76% |
| 21.0 | 100.00% |
| 21.4 | 9.44% |
| 21.9 | 5.79% |
| 22.6 | 2.79% |
| 23.1 | 26.82% |
| 23.7 | 9.43% |
| 24.8 | 4.19% |
| 25.3 | 5.42% |
| 25.4 | 5.79% |
| 26.3 | 4.18% |
| 27.0 | 2.23% |
| 28.2 | 1.76% |
| 29.1 | 1.74% |
| 30.4 | 4.86% |
| 32.0 | 3.82% |
| 33.9 | 2.36% |

In embodiments, monofumarate salt of Compound 1 is characterized by one, two, three, four, five, six, seven, eight, nine, ten, eleven, or more, XRPD peaks listed in Table D.

In embodiments, the monofumarate salt of Compound 1 of Form 4 has a chemical purity of greater than about 80%, about 85%, about 90%, about 95%, about 96%, about 97%, about 98%, about 99%, 99.5%, or about 100% by weight as determined by HPLC.

### Form 5:

In embodiments, the monofumarate salt of Compound 1, is characterized by an XRPD pattern having peaks at 8.7±0.2, 19.7±0.2, and 23.5±0.2 °2θ. In embodiments, the variance at any of these peaks is ±0.1 °2θ.

In embodiments, the monofumarate salt of Compound 1 is characterized by an XRPD having peaks at 8.7±0.2, 19.7±0.2, and 23.5±0.2 °2θ and at least one (e.g., 1, 2, 3, 4, or 5) peak in an XRPD pattern selected from 14.4±0.2, 18.7±0.2, 19.2±0.2, 21.8±0.2, and 23.2±0.2 °2θ. In embodiments, the variance at any of these peaks is ±0.1 °2θ.

In embodiments, the monofumarate salt of Compound 1 is characterized by an XRPD having peaks at 8.7±0.2, 14.4±0.2, 18.7±0.2, 19.2±0.2, 19.7±0.2, 21.8±0.2, 23.2±0.2, and 23.5±0.2 °2θ. In embodiments, the variance at any of these peaks is ±0.1 °2θ.

In embodiments, the monofumarate salt of Compound 1 is characterized by an XRPD pattern having one or more peaks at 7.1±0.2, 7.8±0.2, 8.7±0.2, 9.5±0.2, 10.5±0.2, 11.1±0.2, 11.6±0.2, 12.2±0.2, 12.8±0.2, 14.4±0.2, 15.6±0.2, 16.4±0.2, 16.8±0.2, 17.4±0.2, 17.7±0.2, 18.7±0.2, 19.2±0.2, 19.7±0.2, 20.0±0.2, 20.8±0.2, 21.1±0.2, 21.4±0.2, 21.8±0.2, 22.2±0.2, 22.8±0.2, 23.2±0.2, 23.5±0.2, 24.6±0.2, 25.2±0.2, 26.4±0.2, 26.7±0.2, 28.2±0.2, 29.0±0.2, 33.3±0.2, and 34.8±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3K.

In embodiments, monofumarate salt of Compound 1 is characterized by an XRPD pattern comprising peaks shown in Table E.

TABLE E
XRPD Peak Positions for the Monofumarate
Salt of Compound 1 (“Form 5”)

| Peak Position °2θ | Relative Intensity |
|---|---|
| 7.1 | 12.12% |
| 7.8 | 14.56% |
| 8.7 | 100.00% |
| 9.5 | 2.42% |
| 10.5 | 21.83% |
| 11.1 | 7.58% |
| 11.6 | 6.80% |
| 12.2 | 19.22% |
| 12.8 | 17.71% |
| 14.4 | 38.88% |
| 15.6 | 10.92% |
| 16.4 | 4.91% |
| 16.8 | 14.28% |
| 17.4 | 18.84% |
| 17.7 | 8.77% |
| 18.7 | 45.47% |
| 19.2 | 48.87% |
| 19.7 | 56.75% |
| 20.0 | 18.65% |
| 20.8 | 11.74% |
| 21.1 | 13.79% |
| 21.4 | 18.35% |
| 21.8 | 26.15% |
| 22.2 | 21.53% |
| 22.8 | 16.61% |
| 23.2 | 33.61% |
| 23.5 | 54.79% |
| 24.6 | 8.57% |
| 25.2 | 16.09% |
| 26.4 | 11.70% |
| 26.7 | 4.25% |
| 28.2 | 3.69% |
| 29.0 | 20.56% |
| 33.3 | 3.27% |
| 34.8 | 4.14% |

In embodiments, monofumarate salt of Compound 1 is characterized by one, two, three, four, five, six, seven, eight, nine, ten, eleven, or more, XRPD peaks listed in Table E.

In embodiments, the monofumarate salt of Compound 1 of Form 5 has a chemical purity of greater than about 80%, about 85%, about 90%, about 95%, about 96%, about 97%, about 98%, about 99%, 99.5%, or about 100% by weight as determined by HPLC.

### Form 6:

In embodiments, the monofumarate salt of Compound 1, is characterized by an XRPD pattern having peaks at 7.3±0.2, 10.6±0.2, and 19.8±0.2 °2θ. In embodiments, the variance at any of these peaks is ±0.1 °2θ.

In embodiments, the monofumarate salt of Compound 1 is characterized by an XRPD having peaks at 7.3±0.2, 10.6±0.2, and 19.8±0.2 °2θ and at least one (e.g., 1, 2, 3, 4, or 5) peak in an XRPD pattern selected from 13.9±0.2, 18.0 0.2, 20.4±0.2, 23.1±0.2, and 23.6±0.2 °2θ. In embodiments, the variance at any of these peaks is ±0.1 °2θ.

In embodiments, the monofumarate salt of Compound 1 is characterized by an XRPD having peaks at 7.3±0.2, 10.6±0.2, 13.9±0.2, 18.0±0.2, 19.8±0.2, 20.4±0.2, 23.1±0.2, and 23.6±0.2 °2θ. In embodiments, the variance at any of these peaks is ±0.1 °2θ.

In embodiments, the monofumarate salt of Compound 1 is characterized by an XRPD pattern having one or more peaks at 7.3±0.2, 8.0 0.2, 8.4±0.2, 8.9±0.2, 10.1±0.2, 10.6±0.2, 11.6±0.2, 12.1±0.2, 12.4±0.2, 13.7±0.2, 13.9±0.2, 14.6±0.2, 15.1±0.2, 15.5±0.2, 15.8±0.2, 16.1±0.2, 16.3±0.2, 18.0±0.2, 18.3±0.2, 18.8±0.2, 19.2±0.2, 19.8±0.2, 20.4±0.2, 20.9±0.2, 21.8±0.2, 22.2±0.2, 22.5±0.2, 23.1±0.2, 23.6±0.2, 24.0±0.2, 24.6±0.2, 25.1±0.2, 25.7±0.2, 26.1±0.2, 26.9±0.2, 28.3±0.2, 29.2±0.2, 31.0±0.2, 31.6±0.2, 33.2±0.2, 34.9±0.2, and 36.8±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3L.

In embodiments, monofumarate salt of Compound 1 is characterized by an XRPD pattern comprising peaks shown in Table F.

TABLE F
XRPD Peak Positions for the Monofumarate
Salt of Compound 1 (“Form 6”)

| Peak Position °2θ | Relative Intensity |
|---|---|
| 7.3 | 100.00% |
| 8.0 | 6.05% |
| 8.4 | 8.35% |
| 8.9 | 5.97% |
| 10.1 | 2.72% |
| 10.6 | 66.01% |
| 11.6 | 32.67% |
| 12.1 | 11.78% |
| 12.4 | 11.73% |
| 13.7 | 7.61% |
| 13.9 | 28.93% |
| 14.6 | 5.58% |
| 15.1 | 5.35% |
| 15.5 | 11.30% |
| 15.8 | 18.41% |
| 16.1 | 16.74% |
| 16.3 | 5.10% |
| 18.0 | 30.94% |
| 18.3 | 18.86% |
| 18.8 | 20.43% |
| 19.2 | 22.58% |
| 19.8 | 52.24% |
| 20.4 | 43.15% |
| 20.9 | 25.38% |
| 21.8 | 9.21% |
| 22.2 | 20.58% |
| 22.5 | 27.93% |
| 23.1 | 44.36% |
| 23.6 | 42.49% |
| 24.0 | 33.74% |
| 24.6 | 8.01% |
| 25.1 | 11.15% |
| 25.7 | 19.37% |
| 26.1 | 6.26% |
| 26.9 | 5.22% |
| 28.3 | 4.93% |
| 29.2 | 10.71% |
| 31.0 | 5.21% |
| 31.6 | 4.13% |
| 33.2 | 2.32% |
| 34.9 | 3.02% |
| 36.8 | 2.57% |

In embodiments, monofumarate salt of Compound 1 is characterized by one, two, three, four, five, six, seven, eight, nine, ten, eleven, or more, XRPD peaks listed in Table F.

In embodiments, the monofumarate salt of Compound 1 of Form 6 has a chemical purity of greater than about 80%, about 85%, about 90%, about 95%, about 96%, about 97%, about 98%, about 99%, 99.5%, or about 100% by weight as determined by HPLC.

### Form 7:

In embodiments, the monofumarate salt of Compound 1, is characterized by an XRPD pattern having peaks at 18.7±0.2, 23.6±0.2, and 25.2±0.2 °2θ. In embodiments, the variance at any of these peaks is ±0.1 °2θ.

In embodiments, the monofumarate salt of Compound 1 is characterized by an XRPD having peaks at 18.7±0.2, 23.6±0.2, and 25.2±0.2 °2θ and at least one (e.g., 1, 2, 3, 4, or 5) peak in an XRPD pattern selected from 8.7±0.2, 10.5±0.2, 17.2±0.2, 19.7±0.2, and 21.4±0.2 °2θ. In embodiments, the variance at any of these peaks is ±0.1 °2θ.

In embodiments, the monofumarate salt of Compound 1 is characterized by an XRPD having peaks at 8.7±0.2, 10.5±0.2, 17.2±0.2, 18.7±0.2, 19.7±0.2, 21.4±0.2, 23.6±0.2, and 25.2±0.2 °2θ. In embodiments, the variance at any of these peaks is ±0.1 °2θ.

In embodiments, the monofumarate salt of Compound 1 is characterized by an XRPD pattern having one or more peaks at 5.3±0.2, 7.2±0.2, 8.7±0.2, 8.9±0.2, 10.2±0.2, 10.5±0.2, 11.5±0.2, 11.8±0.2, 12.2±0.2, 12.8±0.2, 14.1±0.2, 15.2±0.2, 16.5±0.2, 17.2±0.2, 17.9±0.2, 18.2±0.2, 18.7±0.2, 19.4±0.2, 19.7±0.2, 20.8±0.2, 21.1±0.2, 21.4±0.2, 21.9±0.2, 23.2±0.2, 23.6±0.2, 24.0±0.2, 24.9±0.2, 25.2±0.2, 26.4±0.2, 27.5±0.2, 29.2±0.2, 30.3±0.2, and 38.1±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3M.

In embodiments, monofumarate salt of Compound 1 is characterized by an XRPD pattern comprising peaks shown in Table G.

TABLE G
XRPD Peak Positions for the Monofumarate
Salt of Compound 1 (“Form 7”)

| Peak Position °2θ | Relative Intensity |
|---|---|
| 5.3 | 22.31% |
| 7.2 | 31.71% |
| 8.7 | 59.38% |
| 8.9 | 24.91% |
| 10.2 | 34.92% |
| 10.5 | 66.98% |
| 11.5 | 5.99% |
| 11.8 | 11.81% |
| 12.2 | 16.23% |
| 12.8 | 22.05% |
| 14.1 | 21.01% |
| 15.2 | 26.42% |
| 16.5 | 15.17% |
| 17.2 | 80.05% |
| 17.9 | 51.20% |
| 18.2 | 54.23% |
| 18.7 | 90.34% |
| 19.4 | 54.33% |
| 19.7 | 76.74% |
| 20.8 | 12.45% |
| 21.1 | 32.13% |
| 21.4 | 75.17% |
| 21.9 | 48.60% |
| 23.2 | 67.15% |
| 23.6 | 100.00% |
| 24.0 | 27.37% |
| 24.9 | 25.35% |
| 25.2 | 86.24% |
| 26.4 | 25.54% |
| 27.5 | 6.78% |
| 29.2 | 15.66% |
| 30.3 | 8.66% |
| 38.1 | 6.21% |

In embodiments, monofumarate salt of Compound 1 is characterized by one, two, three, four, five, six, seven, eight, nine, ten, eleven, or more, XRPD peaks listed in Table G.

In embodiments, the monofumarate salt of Compound 1 of Form 7 has a chemical purity of greater than about 80%, about 85%, about 90%, about 95%, about 96%, about 97%, about 98%, about 99%, 99.5%, or about 100% by weight as determined by HPLC.

### Form 8:

In embodiments, the monofumarate salt of Compound 1, is characterized by an XRPD pattern having peaks at 5.6±0.2, 11.2±0.2, and 19.7±0.2 °2θ. In embodiments, the variance at any of these peaks is ±0.1 °2θ.

In embodiments, the monofumarate salt of Compound 1 is characterized by an XRPD having peaks at 5.6±0.2, 11.2±0.2, and 19.7±0.2 °2θ and at least one (e.g., 1, 2, 3, 4, or 5) peak in an XRPD pattern selected from 9.3±0.2, 16.8±0.2, 18.6±0.2, 21.1±0.2, and 23.5±0.2 °2θ. In embodiments, the variance at any of these peaks is ±0.1 °2θ.

In embodiments, the monofumarate salt of Compound 1 is characterized by an XRPD having peaks at 5.6±0.2, 9.3±0.2, 11.2±0.2, 16.8±0.2, 18.6±0.2, 19.7±0.2, 21.1±0.2, and 23.5±0.2 °2θ. In embodiments, the variance at any of these peaks is ±0.1 °2θ.

In embodiments, the monofumarate salt of Compound 1 is characterized by an XRPD pattern having one or more peaks at 5.6±0.2, 7.1±0.2, 8.1±0.2, 8.7±0.2, 9.3±0.2, 10.4±0.2, 11.2±0.2, 12.7±0.2, 13.2±0.2, 14.4±0.2, 15.0±0.2, 15.8±0.2, 16.8±0.2, 18.6±0.2, 19.3±0.2, 19.7±0.2, 20.8±0.2, 21.1±0.2, 21.8±0.2, 22.5±0.2, 23.2±0.2, 23.5±0.2, 25.1±0.2, 29.1±0.2, and 29.5±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3N.

In embodiments, monofumarate salt of Compound 1 is characterized by an XRPD pattern comprising peaks shown in Table H.

TABLE H
XRPD Peak Positions for the Monofumarate
Salt of Compound 1 (“Form 8”)

| Peak Position °2θ | Relative Intensity |
|---|---|
| 5.6 | 88.19% |
| 7.1 | 3.37% |
| 8.1 | 14.51% |
| 8.7 | 12.42% |
| 9.3 | 19.45% |
| 10.4 | 4.86% |
| 11.2 | 100.00% |
| 12.7 | 7.25% |
| 13.2 | 6.64% |
| 14.4 | 3.39% |
| 15.0 | 3.65% |
| 15.8 | 9.17% |
| 16.8 | 24.21% |
| 18.6 | 19.48% |
| 19.3 | 18.39% |
| 19.7 | 30.58% |
| 20.8 | 5.96% |
| 21.1 | 20.26% |
| 21.8 | 5.52% |
| 22.5 | 3.58% |
| 23.2 | 8.38% |
| 23.5 | 21.51% |
| 25.1 | 10.17% |
| 29.1 | 2.58% |
| 29.5 | 3.34% |

In embodiments, monofumarate salt of Compound 1 is characterized by one, two, three, four, five, six, seven, eight, nine, ten, eleven, or more, XRPD peaks listed in Table H.

In embodiments, the monofumarate salt of Compound 1 of Form 8 has a chemical purity of greater than about 80%, about 85%, about 90%, about 95%, about 96%, about 97%, about 98%, about 99%, 99.5%, or about 100% by weight as determined by HPLC.

### Form 9:

In embodiments, the monofumarate salt of Compound 1, is characterized by an XRPD pattern having peaks at 10.8±0.2, 16.2±0.2, and 21.6±0.2 °2θ. In embodiments, the variance at any of these peaks is ±0.1 °2θ.

In embodiments, the monofumarate salt of Compound 1 is characterized by an XRPD having peaks at 10.8±0.2, 16.2±0.2, and 21.6±0.2 °2θ and at least one (e.g., 1, 2, 3, 4, or 5) peak in an XRPD pattern selected from 5.5±0.2, 12.1±0.2, 17.7±0.2, 18.2±0.2, and 22.7±0.2 °2θ. In embodiments, the variance at any of these peaks is ±0.1 °2θ.

In embodiments, the monofumarate salt of Compound 1 is characterized by an XRPD having peaks at 5.5±0.2, 10.8±0.2, 12.1±0.2, 16.2±0.2, 17.7±0.2, 18.2±0.2, 21.6±0.2, and 22.7±0.2 °2θ. In embodiments, the variance at any of these peaks is ±0.1 °2θ.

In embodiments, the monofumarate salt of Compound 1 is characterized by an XRPD pattern having one or more peaks at 5.5±0.2, 6.1±0.2, 8.2±0.2, 8.8±0.2, 9.6±0.2, 10.0±0.2, 10.8±0.2, 11.4±0.2, 12.1±0.2, 12.9±0.2, 13.2±0.2, 14.6±0.2, 16.2±0.2, 17.0±0.2, 17.7±0.2, 18.2±0.2, 19.1±0.2, 19.9±0.2, 20.4±0.2, 20.7±0.2, 21.6±0.2, 22.7±0.2, 23.5±0.2, 24.2±0.2, 25.3±0.2, 27.9±0.2, 28.6±0.2, 29.5±0.2, 32.5±0.2, and 33.3±0.2 °2θ. In embodiments, the variance at any of these peaks is ±0.1 °2θ. In embodiments, the monofumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the monofumarate salt of Compound 1 is characterized by an XRPD pattern substantially similar to FIG. 3O.

In embodiments, monofumarate salt of Compound 1 is characterized by an XRPD pattern comprising peaks shown in Table I.

TABLE I
XRPD Peak Positions for the Monofumarate
Salt of Compound 1 (“Form 9”)

| Peak Position °2θ | Relative Intensity |
|---|---|
| 5.5 | 16.11% |
| 6.1 | 1.13% |
| 8.2 | 3.71% |
| 8.8 | 1.57% |
| 9.6 | 1.45% |
| 10.0 | 3.67% |
| 10.8 | 48.16% |
| 11.4 | 6.55% |
| 12.1 | 10.24% |
| 12.9 | 2.55% |
| 13.2 | 1.42% |
| 14.6 | 1.34% |
| 16.2 | 100.00% |
| 17.0 | 7.88% |
| 17.7 | 11.69% |
| 18.2 | 14.50% |
| 19.1 | 1.64% |
| 19.9 | 8.68% |
| 20.4 | 5.27% |
| 20.7 | 3.44% |
| 21.6 | 36.48% |
| 22.7 | 17.27% |
| 23.5 | 4.20% |
| 24.2 | 4.25% |
| 25.3 | 2.97% |
| 27.9 | 3.10% |
| 28.6 | 1.31% |
| 29.5 | 0.61% |
| 32.5 | 2.75% |
| 33.3 | 1.28% |

In embodiments, monofumarate salt of Compound 1 is characterized by one, two, three, four, five, six, seven, eight, nine, ten, eleven, or more, XRPD peaks listed in Table I.

In embodiments, the monofumarate salt of Compound 1 of Form 9 has a chemical purity of greater than about 80%, about 85%, about 90%, about 95%, about 96%, about 97%, about 98%, about 99%, 99.5%, or about 100% by weight as determined by HPLC.

In embodiments, the present provides a sesquifumarate salt of Compound 1. In embodiments the sesquifumarate salt of Compound 1 has the following formula:

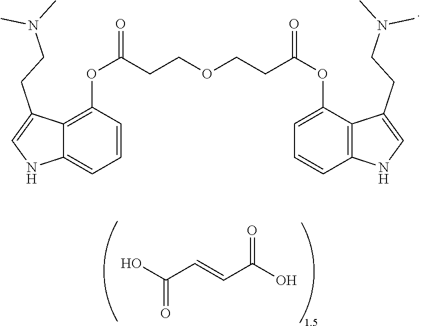

In embodiments, the sesquifumarate salt of Compound 1 is crystalline.

In embodiments, the sesquifumarate salt of Compound 1 is characterized by an XRPD pattern having peaks at 8.7±0.2, 19.7±0.2, and 23.6±0.2 °2θ.

In embodiments, the sesquifumarate salt of Compound 1 is characterized by an XRPD pattern having peaks at 8.7±0.2, 19.7±0.2, and 23.6±0.2 °2θ and at least one (e.g., 1, 2, or 3) peak in an XRPD pattern selected from 14.4±0.2, 21.8±0.2, and 22.2±0.2° 26. In embodiments, the variance at any of these peaks is ±0.1 °2θ.

In embodiments, the sesquifumarate salt of Compound 1 is characterized by an XRPD pattern having peaks at 8.7±0.2, 14.4±0.2, 19.7±0.2, 21.8±0.2, 22.2±0.2, 23.6±0.2 °2θ. In embodiments, the variance at any of these peaks is ±0.1 °2θ.

In embodiments, the sesquifumarate salt of Compound 1 is characterized by an XRPD pattern having one or more peaks at 7.9±0.2, 8.7±0.2, 9.6±0.2, 11.1±0.2, 11.6±0.2, 12.3±0.2, 14.0±0.2, 14.4±0.2, 15.6±0.2, 16.3±0.2, 16.8±0.2, 17.2±0.2, 17.4±0.2, 17.8±0.2, 18.0±0.2, 18.4±0.2, 18.7±0.2, 19.2±0.2, 19.7±0.2, 20.1±0.2, 20.8±0.2, 21.1±0.2, 21.4±0.2, 21.8±0.2, 22.2±0.2, 22.5±0.2, 22.9±0.2, 23.2±0.2, 23.6±0.2, 24.3±0.2, 24.6±0.2, 25.3±0.2, 26.4±0.2, 26.8±0.2, 27.4±0.2, 28.2±0.2, 29.0±0.2, 29.5±0.2, 29.7±0.2, 30.1±0.2, 30.4±0.2, 30.9±0.2, 31.6±0.2, 33.2±0.2, 34.0±0.2, 34.7±0.2, 34.9±0.2, 35.4±0.2, 38.4, 39.0±0.2, and 39.9±0.2 and °2θ. In embodiments, the sesquifumarate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, or 26 of the aforementioned peaks, or any range therein (e.g., from 1-26, 2-20, 3-15, or 3-10 of the aforementioned peaks).

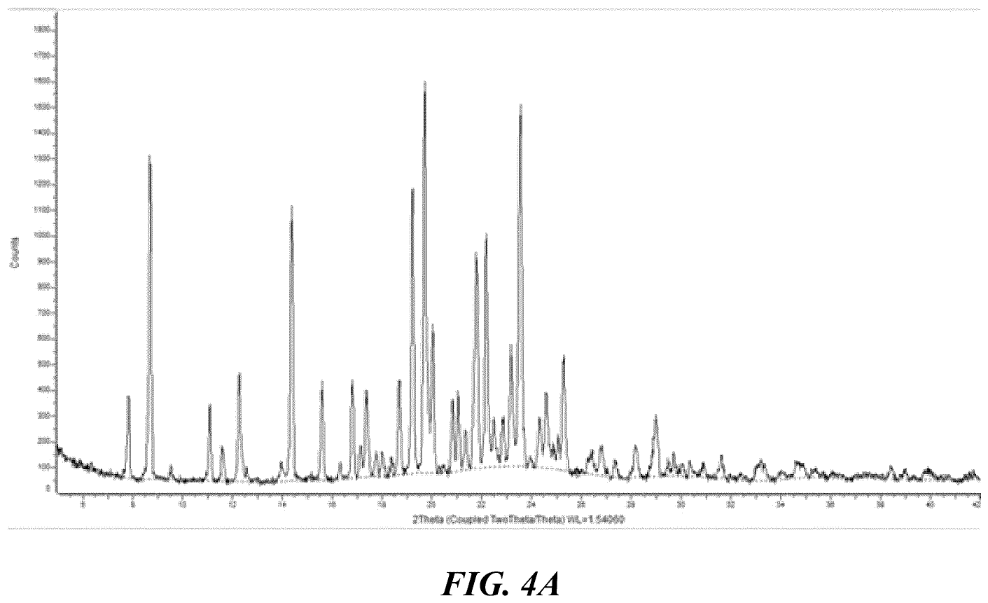

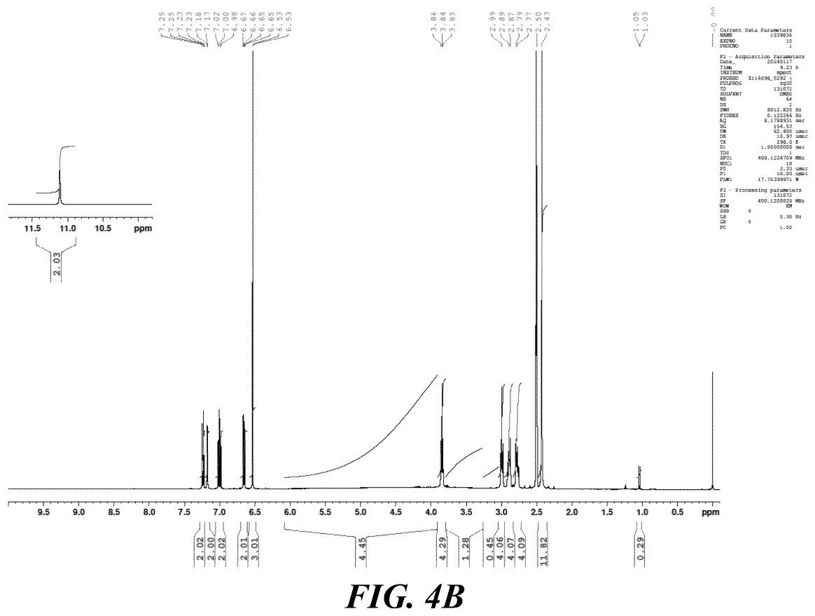

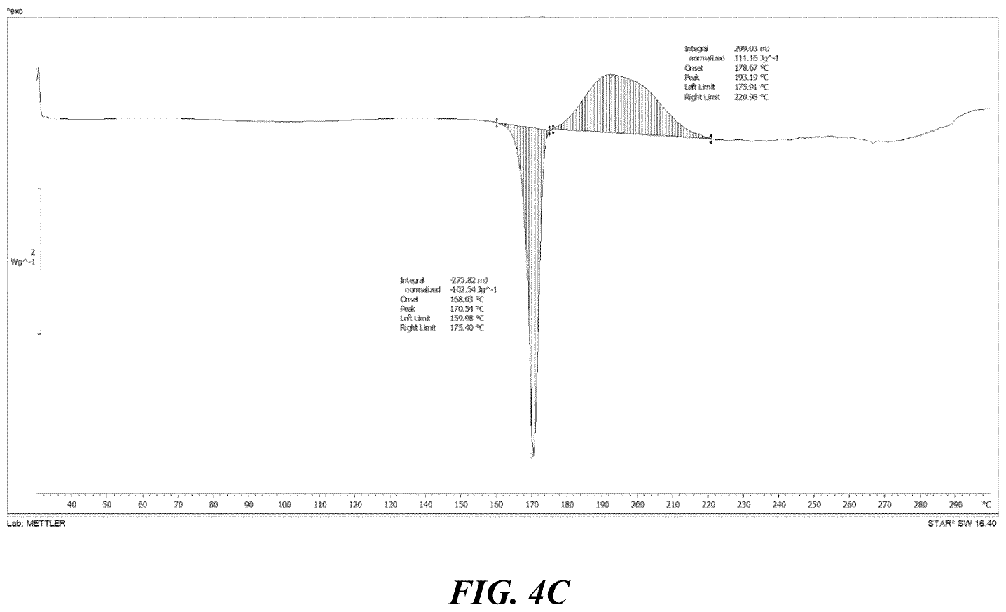

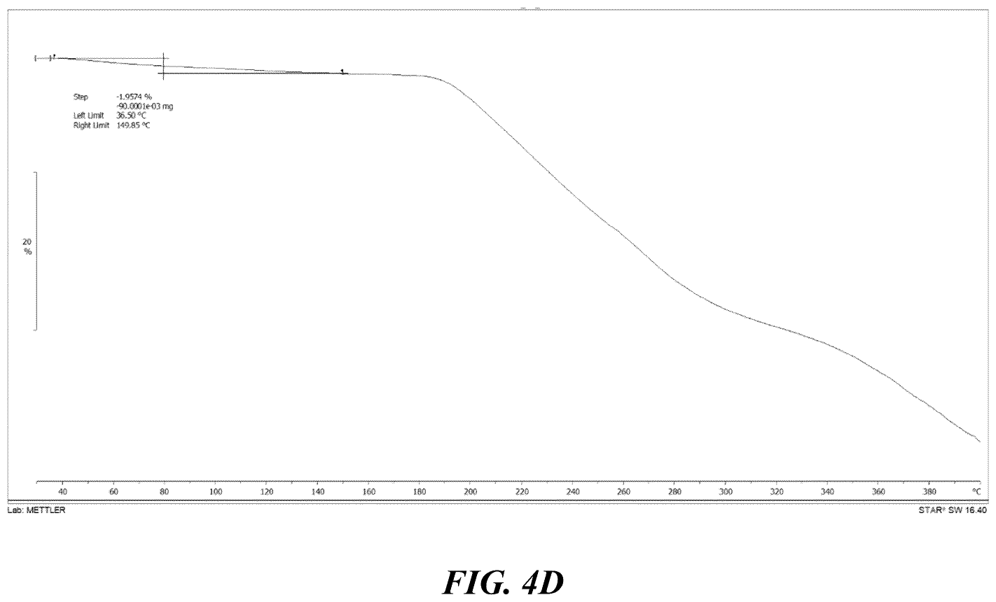

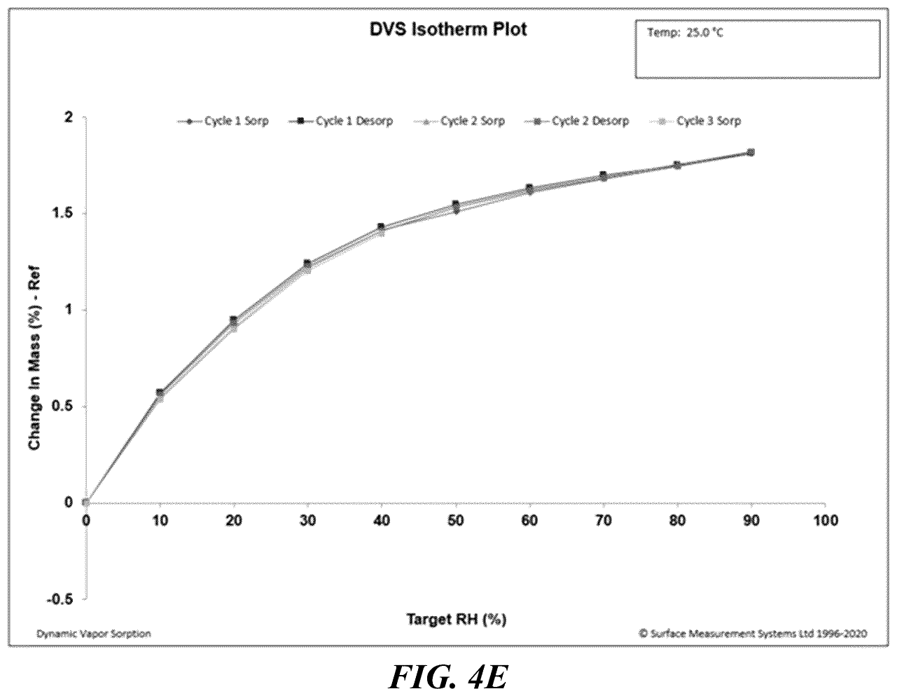

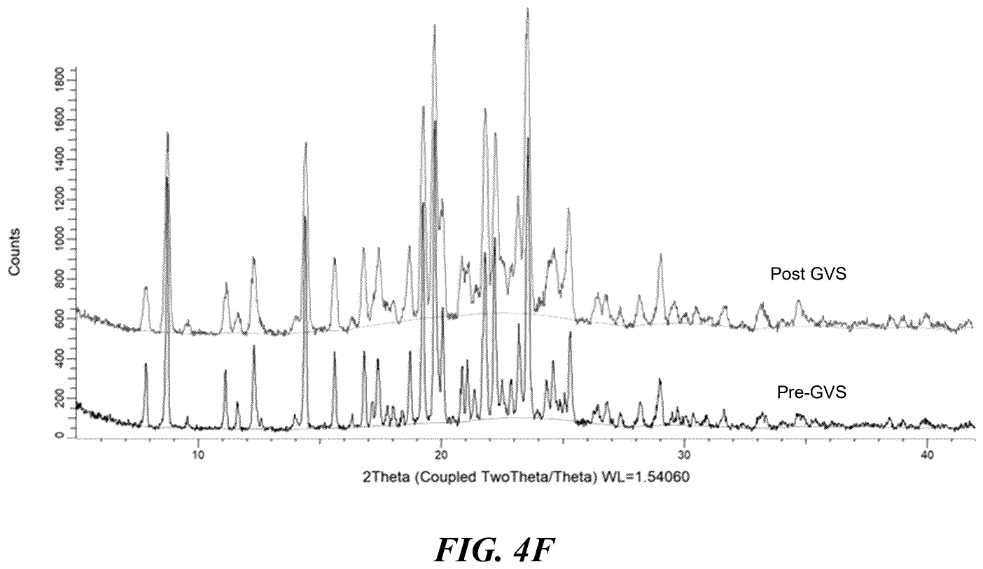

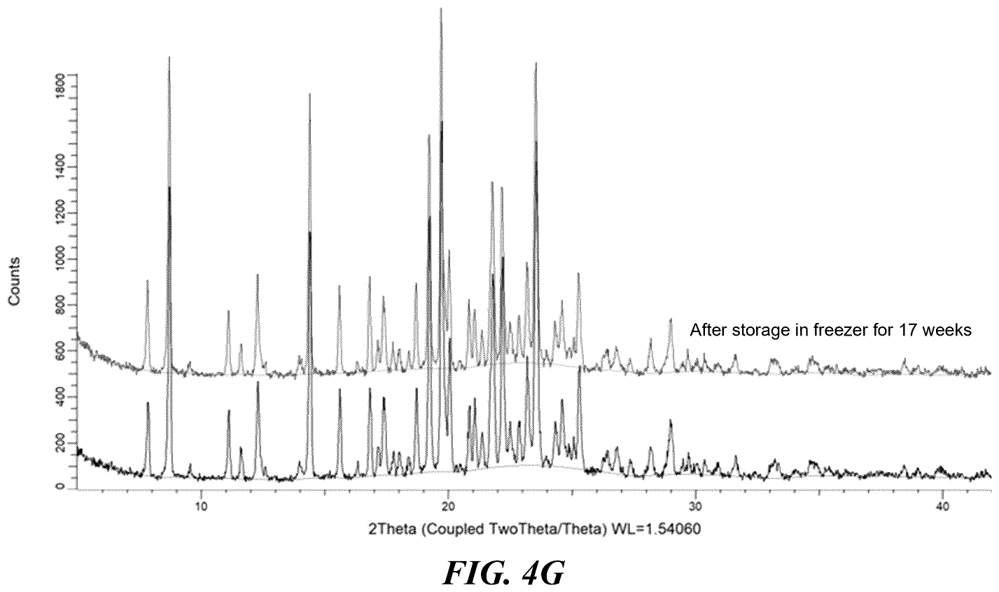

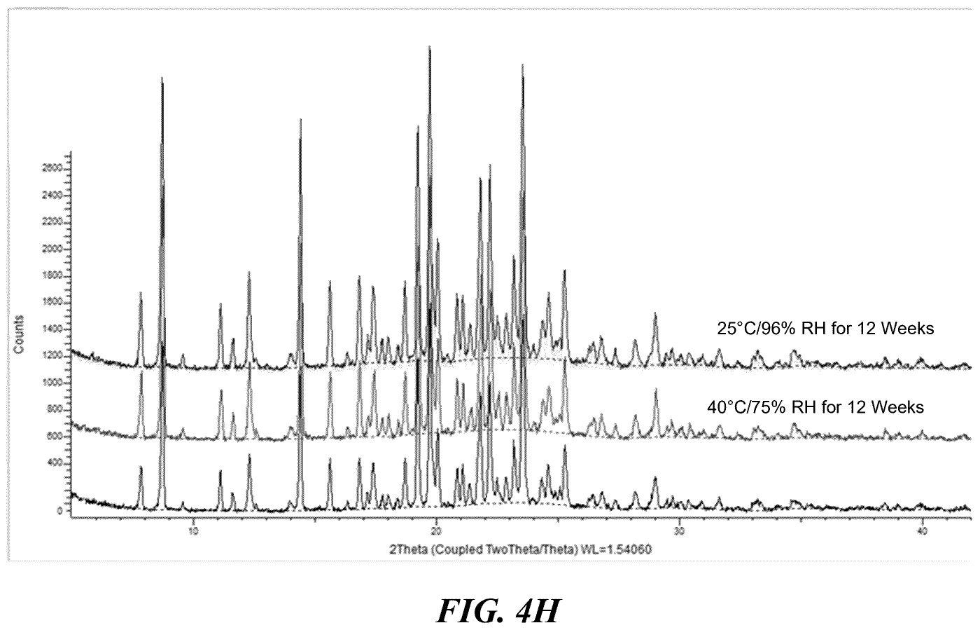

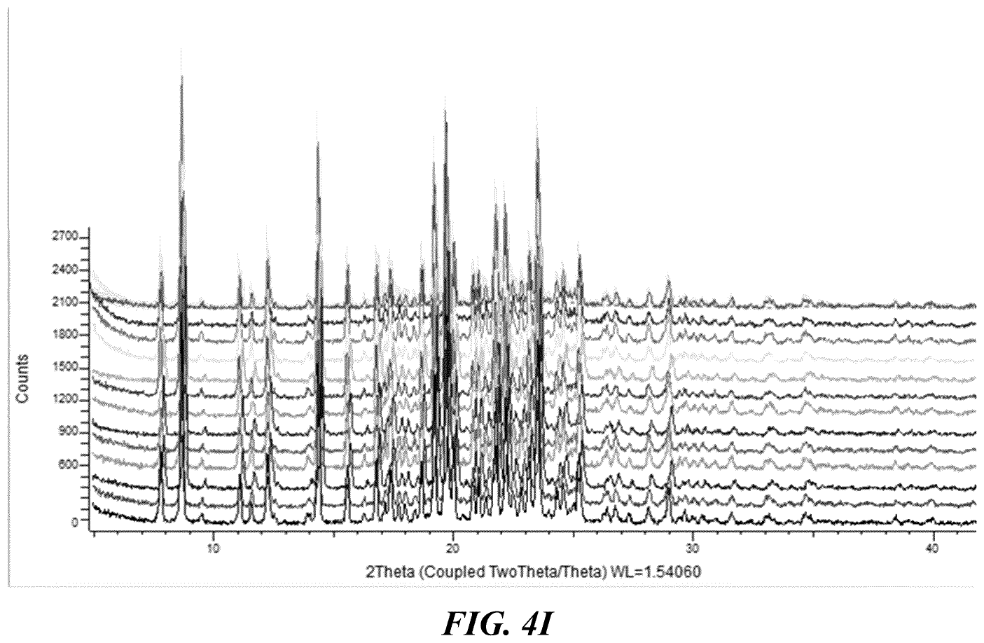

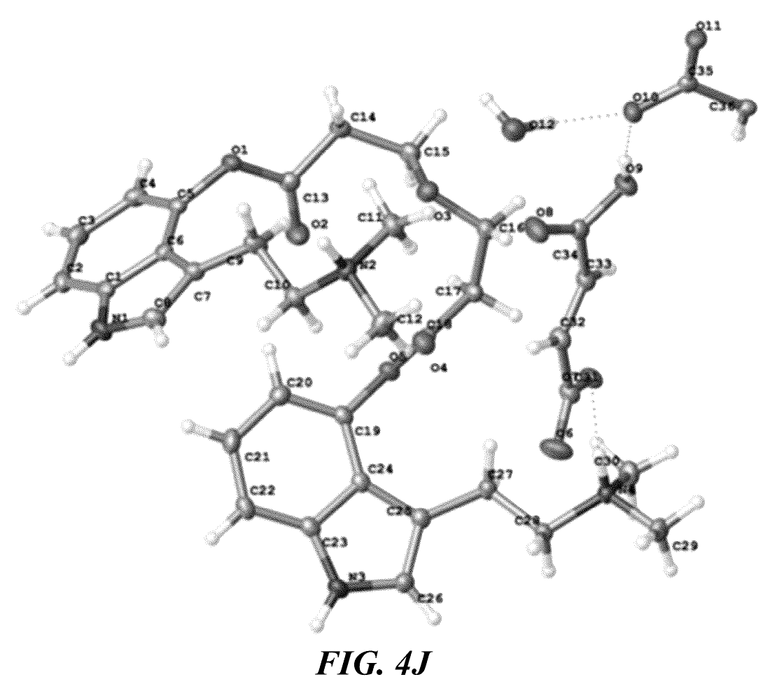

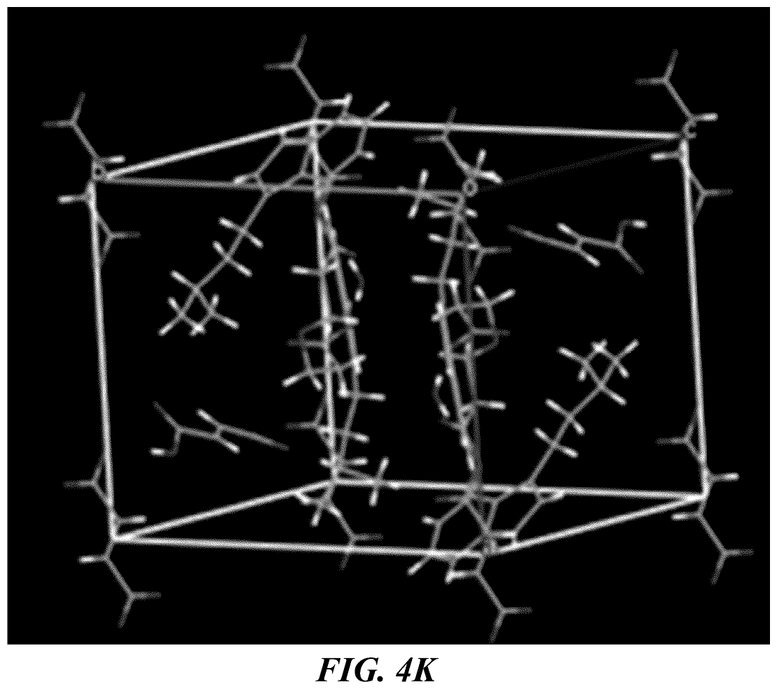

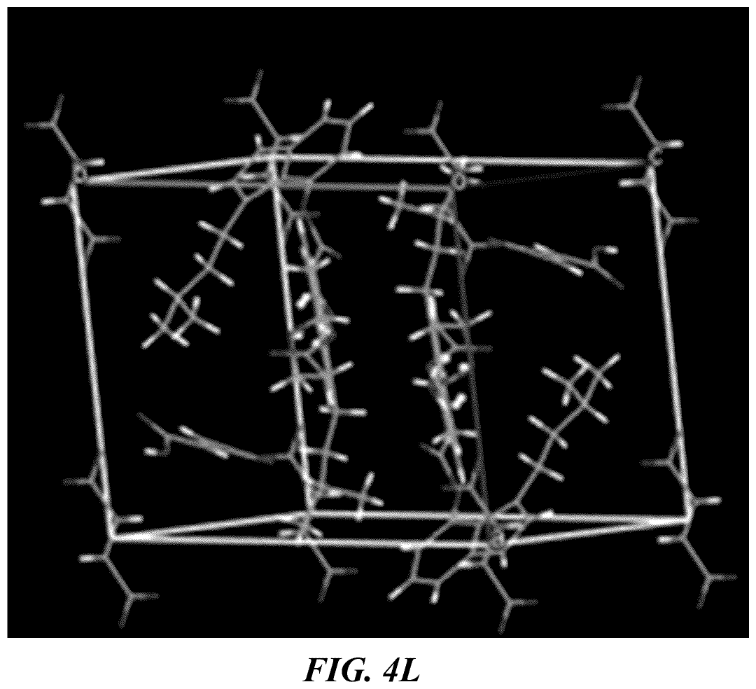

In embodiments, the sesquifumarate salt of Compound 1 is characterized by an XRPD pattern substantially similar to FIG. 4A.

In embodiments, sesquifumarate salt of Compound 1 is characterized by an XRPD pattern comprising peaks shown in Table J.

TABLE J
XRPD Peak Positions for the Sesquifumarate Salt of Compound 1

| Position°2θ | Relative Intensity |
|---|---|
| 7.9 | 18.1% |
| 8.7 | 76.9% |
| 9.6 | 3.5% |
| 11.1 | 18.4% |
| 11.6 | 7.2% |
| 12.3 | 26.4% |
| 14.0 | 4.8% |
| 14.4 | 66.2% |
| 15.6 | 24.7% |
| 16.3 | 4.6% |
| 16.8 | 24.5% |
| 17.2 | 7.3% |
| 17.4 | 18.1% |
| 17.8 | 6.8% |
| 18.0 | 6.1% |
| 18.4 | 5.4% |
| 18.7 | 24.5% |
| 19.2 | 74.0% |
| 19.7 | 100.0% |
| 20.1 | 38.7% |
| 20.8 | 18.2% |
| 21.1 | 21.9% |
| 21.4 | 10.7% |
| 21.8 | 55.6% |
| 22.2 | 60.2% |
| 22.5 | 13.4% |
| 22.9 | 12.6% |
| 23.2 | 30.5% |
| 23.6 | 85.6% |
| 24.3 | 12.3% |
| 24.6 | 20.3% |
| 25.3 | 28.1% |
| 26.4 | 5.9% |
| 26.8 | 7.6% |
| 27.4 | 3.0% |
| 28.2 | 8.2% |
| 29.0 | 14.2% |
| 29.5 | 4.3% |
| 29.7 | 6.8% |
| 30.1 | 3.4% |
| 30.4 | 5.7% |
| 30.9 | 2.1% |
| 31.6 | 7.0% |
| 33.2 | 5.9% |
| 34.0 | 2.5% |
| 34.7 | 2.9% |
| 34.9 | 3.0% |
| 35.4 | 2.3% |
| 38.4 | 3.9% |
| 39.0 | 2.7% |
| 39.9 | 1.5% |

In embodiments, sesquifumarate salt of Compound 1 is characterized by one, two, three, four, five, six, seven, eight, nine, ten, eleven, or more, XRPD peaks listed in Table J.

In embodiments, the sesquifumarate salt of Compound 1 is characterized by a DSC thermogram comprising an endothermic event at 168±5° C.

In embodiments, the sesquifumarate salt of Compound 1 is characterized by a DSC comprising an exothermic event between 176±5° C. to 221±5° C.

In embodiments, the sesquifumarate salt of Compound 1 is characterized by a DSC thermogram substantially similar to FIG. 4C.

In embodiments, the sesquifumarate salt of Compound 1 is characterized by about a 2% weight loss from 37±5° C. to 150±5° C. as determined by TGA.

In embodiments, the sesquifumarate salt of Compound 1 is characterized by a thermogravimetric thermogram substantially similar to FIG. 3D.

In embodiments, the sesquifumarate salt of Compound 1 has a chemical purity of greater than about 80%, about 85%, about 90%, about 95%, about 96%, about 97%, about 98%, about 99%, 99.5%, or about 100% by weight as determined by HPLC.

In embodiments, the sesquifumarate salt of Compound 1 is a hydrate.

In embodiments, the succinate salt of Compound 1 has the following formula:

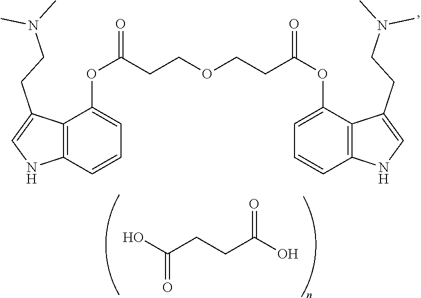

wherein n is from 1 to 2.

In embodiments, n is 1.

In embodiments, n is 1.5.

In embodiments, n is 2.

In embodiments, succinate salt of Compound 1 is crystalline.

In embodiments, succinate salt of Compound 1 is characterized by an XRPD pattern having peaks at 19.7±0.2, 22.1±0.2, and 23.1±0.2 °2θ. In embodiments, the variance at any of these peaks is ±0.1 °2θ.

In embodiments, the succinate salt of Compound 1 is characterized by an XRPD pattern having peaks at 19.7±0.2, 22.1±0.2, and 23.1±0.2 °2θ and at least one (e.g., 1, 2, 3, 4, 5, or 6) peak in an XRPD pattern selected from 8.7±0.2, 14.3±0.2, 16.8±0.2, 18.6±0.2, 19.0±0.2, and 20.2±0.2 °2θ. In embodiments, the variance at any of these peaks is ±0.1 °2θ.

In embodiments, the succinate salt of Compound 1 is characterized by an XRPD pattern having peaks at 8.7±0.2, 14.3±0.2, 16.8±0.2, 18.6±0.2, 19.0±0.2, 19.7±0.2, 20.2±0.2 22.1±0.2, and 23.1±0.2 °2θ. In embodiments, the variance at any of these peaks is ±0.1 °2θ.

In embodiments, the succinate salt of Compound 1 is characterized by an XRPD pattern having one or more peaks at 7.8±0.2, 8.7±0.2, 9.7±0.2, 10.8±0.2, 11.6±0.2, 12.0 0.2, 12.5±0.2, 13.9±0.2, 14.3±0.2, 15.6±0.2, 15.9±0.2, 16.5±0.2, 16.8±0.2, 17.4±0.2, 18.0±0.2, 18.6±0.2, 19.0±0.2, 19.7±0.2, 20.2±0.2, 20.7±0.2, 21.2±0.2, 21.7±0.2, 22.1±0.2, 22.3±0.2, 23.1±0.2, 23.6±0.2, 23.9±0.2, 24.6±0.2, 24.9±0.2, 25.3±0.2, 26.2±0.2, 26.9±0.2, 27.6±0.2, 28.1±0.2, 28.3±0.2, 28.9±0.2, 29.3±0.2, 30.2±0.2, 30.4±0.2, 33.2±0.2, 34.6±0.2, 35.1±0.2, 35.7±0.2, 38.0±0.2, 38.7±0.2, and 39.3±0.2 and °2θ. In embodiments, the variance at any of these peaks is ±0.1 °2θ. In embodiments, the succinate salt of Compound 1 may be characterized by 1, 2, 3, 4, 5, 6, 7, 8, 9, 10, 11, 12, 13, 14, 15, 16, 16, 17, 18, 19, 20, 21, 22, or 23 of the aforementioned peaks, or any range therein (e.g., from 1-23, 2-20, 3-15, or 3-10 of the aforementioned peaks).

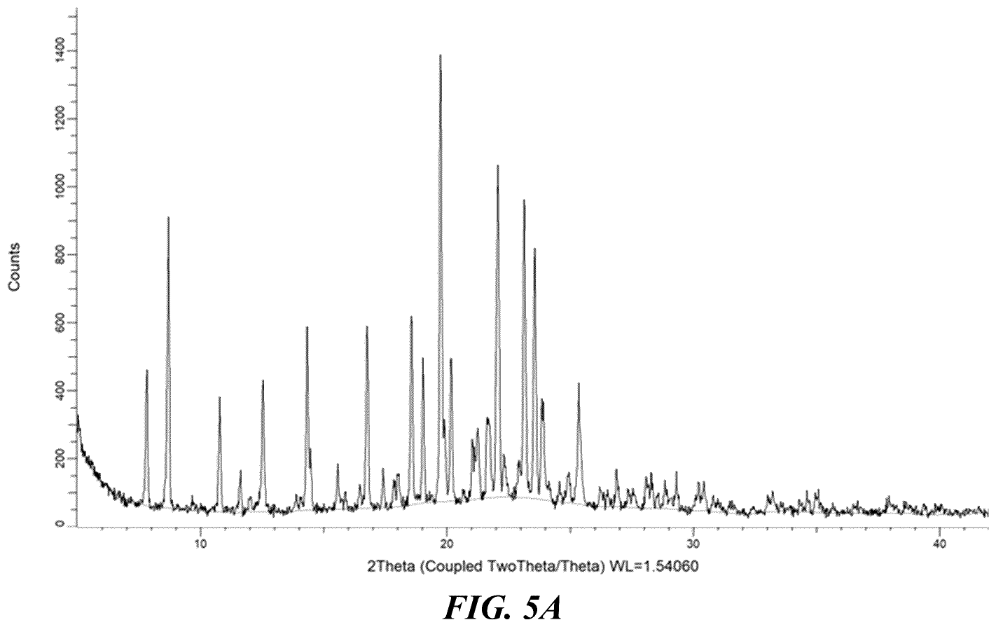

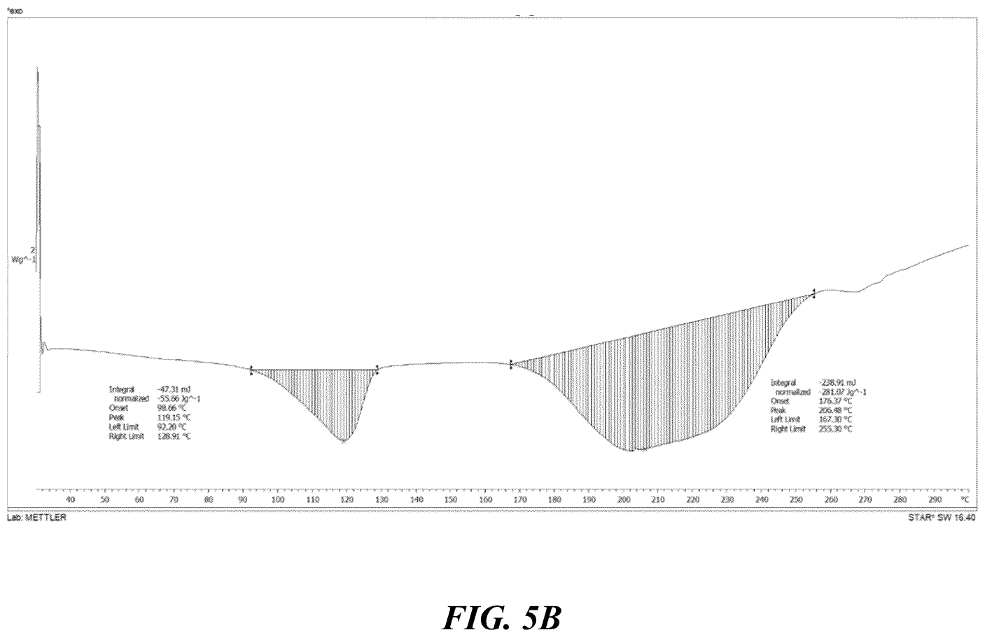

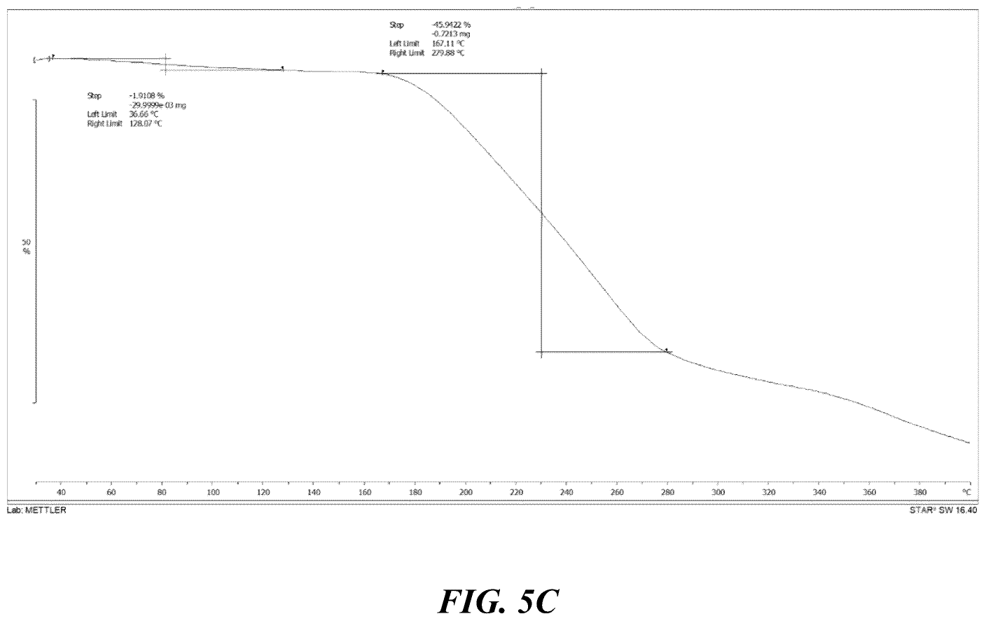

In embodiments, the succinate salt of Compound 1 is characterized by an XRPD pattern substantially similar to FIG. 5A.

In embodiments, succinate salt of Compound 1 is characterized by an XRPD pattern comprising peaks shown in Table K.

TABLE K
XRPD Peak Positions for the Succinate Salt of Compound 1

| Peak position°2θ | Relative Intensity |
|---|---|
| 7.8 | 26.4% |
| 8.7 | 58.2% |
| 9.7 | 1.5% |
| 10.8 | 24.1% |
| 11.6 | 7.9% |
| 12.0 | 2.7% |
| 12.5 | 26.1% |
| 13.9 | 3.9% |
| 14.3 | 39.1% |
| 15.6 | 10.9% |
| 15.9 | 3.3% |
| 16.5 | 5.1% |
| 16.8 | 39.2% |
| 17.4 | 8.9% |
| 18.0 | 6.5% |
| 18.6 | 40.6% |
| 19.0 | 31.9% |
| 19.7 | 100.0% |
| 20.2 | 31.2% |
| 20.7 | 2.1% |
| 21.2 | 15.7% |
| 21.7 | 14.3% |
| 22.1 | 67.9% |
| 22.3 | 8.2% |
| 23.1 | 69.8% |
| 23.6 | 54.4% |
| 23.9 | 23.4% |
| 24.6 | 5.1% |
| 24.9 | 5.4% |
| 25.3 | 31.3% |
| 26.2 | 3.9% |
| 26.9 | 9.2% |
| 27.6 | 4.8% |
| 28.1 | 7.9% |
| 28.3 | 9.2% |
| 28.9 | 6.2% |
| 29.3 | 9.6% |
| 30.2 | 7.2% |
| 30.4 | 6.7% |
| 33.2 | 5.2% |
| 34.6 | 4.6% |
| 35.1 | 3.7% |
| 35.7 | 2.1% |
| 38.0 | 3.2% |
| 38.7 | 1.5% |
| 39.3 | 2.5% |

In embodiments, succinate salt of Compound 1 is characterized by one, two, three, four, five, six, seven, eight, nine, ten, eleven, or more, XRPD peaks listed in Table K.

In embodiments, the succinate salt of Compound 1 is characterized by a DSC thermogram comprising an endothermic event between 92±5° C. to 129±500.

In embodiments, the succinate salt of Compound 1 is characterized by a Differential Scanning Calorimetry (DSC) thermogram comprising an endothermic onset at 99±5° C.

In embodiments, the succinate salt of Compound 1 is characterized by a DSC thermogram substantially similar to FIG. 5B.

In embodiments, the succinate salt of Compound 1 is characterized by about a 2% weight loss from 37±5° C. to 1 28±5° C. as determined by thermal gravimetric analysis (TGA).

In embodiments, the succinate salt of Compound 1 is characterized by a thermogravimetric thermogram substantially similar to FIG. 5C.

In embodiments, the succinate salt of Compound 1 has the following formula:

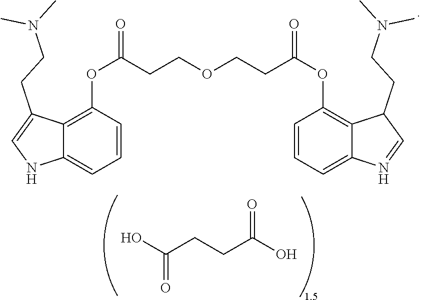

In embodiments, succinate salt of Compound 1 where n=1.5 is characterized by an XRPD pattern having peaks at 8.7±0.2, 18.5±0.2, and 19.7±0.2 °2θ. In embodiments, the variance at any of these peaks is ±0.1 °2θ.

In embodiments, the succinate salt of Compound 1 where n=1.5 is characterized by an XRPD pattern having peaks at 8.7±0.2, 18.5±0.2, and 19.7±0.2 °2θ and at least one (e.g., 1, 2, 3, 4, 5, or 6) peak in an XRPD pattern selected from 7.8±0.2, 14.5±0.2, 23.1±0.2, 23.5±0.2, and 25.3±0.2 °2θ. In embodiments, the variance at any of these peaks is ±0.1 °2θ.

In embodiments, the succinate salt of Compound 1 where n=1.5 is characterized by an XRPD pattern having peaks at 7.8±0.2, 8.7±0.2, 14.5±0.2, 18.5±0.2, 19.7±0.2, 23.1±0.2, 23.5±0.2, and 25.3±0.2 °2θ. In embodiments, the variance at any of these peaks is ±0.1 °2θ.

In embodiments, the succinate salt of Compound 1 where n=1.5 is characterized by an XRPD pattern having one or more peaks at 7.8±0.2, 8.7±0.2, 9.7±0.2, 10.8±0.2, 11.6±0.2, 12.0±0.2, 12.5±0.2, 13.9±0.2, 14.3±0.2, 14.5±0.2, 15.5±0.2, 15.9±0.2, 16.5±0.2, 16.7±0.2, 17.4±0.2, 17.8±0.2, 18.0±0.2, 18.5±0.2, 19.0±0.2, 19.7±0.2, 20.1±0.2, 21.2±0.2, 21.7±0.2, 22.0±0.2, 22.3±0.2, 23.1±0.2, 23.5±0.2, 23.9±0.2, 24.5±0.2, 24.9±0.2, 25.3±0.2, 26.9±0.2, 27.3±0.2, 28.1±0.2, 28.5±0.2, 28.9±0.2, 29.3±0.2, 30.1±0.2, 30.4±0.2, 30.7±0.2, 31.6±0.2, 33.2±0.2, 34.6±0.2, 34.9±0.2, 35.6±0.2, 36.6±0.2, 37.9±0.2, 38.6±0.2, and 39.3±0.2 °2θ. In embodiments, the variance at any of these peaks is ±0.1 °2θ. In embodiments, the succinate salt of Compound 1 may be characterized by 1, 2, 3, 4, 5, 6, 7, 8, 9, 10, 11, 12, 13, 14, 15, 16, 16, 17, 18, 19, 20, 21, 22, or 23 of the aforementioned peaks, or any range therein (e.g., from 1-23, 2-20, 3-15, or 3-10 of the aforementioned peaks).

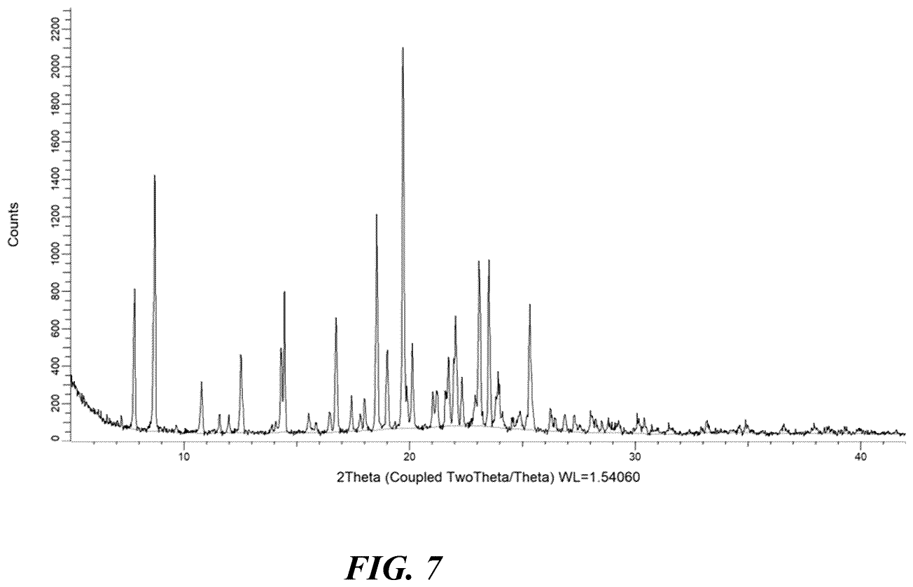

In embodiments, the succinate salt of Compound 1 where n=1.5 is characterized by an XRPD pattern substantially similar to FIG. 7.

In embodiments, succinate salt of Compound 1 where n=1.5 is characterized by an XRPD pattern comprising peaks shown in Table L.

TABLE L
XRPD Peak Positions for the Succinate
(1.5 equivalents) Salt of Compound 1

| Peak position°2θ | Relative Intensity |
|---|---|
| 7.8 | 31.9% |
| 8.7 | 62.4% |
| 9.7 | 1.7% |
| 10.8 | 12.4% |
| 11.6 | 4.3% |
| 12.0 | 4.5% |
| 12.5 | 18.6% |
| 13.9 | 1.8% |
| 14.3 | 19.8% |
| 14.5 | 35.9% |
| 15.5 | 4.8% |
| 15.9 | 1.9% |
| 16.5 | 4.1% |
| 16.7 | 26.6% |
| 17.4 | 8.9% |
| 17.8 | 4.4% |
| 18.0 | 7.9% |
| 18.5 | 55.8% |
| 19.0 | 18.7% |
| 19.7 | 100.0% |
| 20.1 | 22.8% |
| 21.2 | 7.3% |
| 21.7 | 19.2% |
| 22.0 | 29.0% |
| 23.1 | 44.1% |
| 23.5 | 48.3% |
| 23.9 | 15.8% |
| 24.5 | 3.8% |
| 24.9 | 4.4% |
| 25.3 | 35.1% |
| 26.9 | 4.4% |
| 27.3 | 4.9% |
| 28.1 | 2.7% |
| 28.5 | 3.4% |
| 28.9 | 1.2% |
| 29.3 | 3.2% |
| 30.1 | 2.6% |
| 30.4 | 4.6% |
| 30.7 | 2.1% |
| 31.6 | 1.9% |
| 33.2 | 3.7% |
| 34.6 | 1.9% |
| 34.9 | 3.3% |
| 35.6 | 0.8% |
| 36.6 | 2.8% |
| 37.9 | 2.4% |
| 38.6 | 1.9% |
| 39.3 | 1.9% |

In embodiments, the succinate salt of Compound 1 has a chemical purity of greater than about 80%, about 85%, about 90%, about 95%, about 96%, about 97%, about 98%, about 99%, 99.5%, or about 100% by weight as determined by HPLC.

In embodiments, the present disclosure provides a L-tartrate salt of Compound 1 having the following chemical formula:

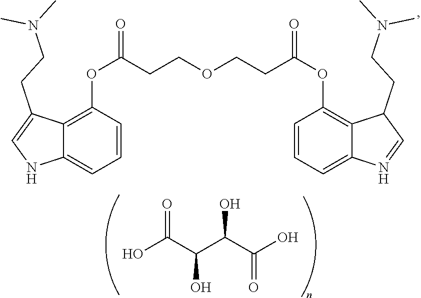

wherein n ranges from 1 to 2.

In embodiments, n is 1.

In embodiments, n is 1.5.

In embodiments, n is 2.

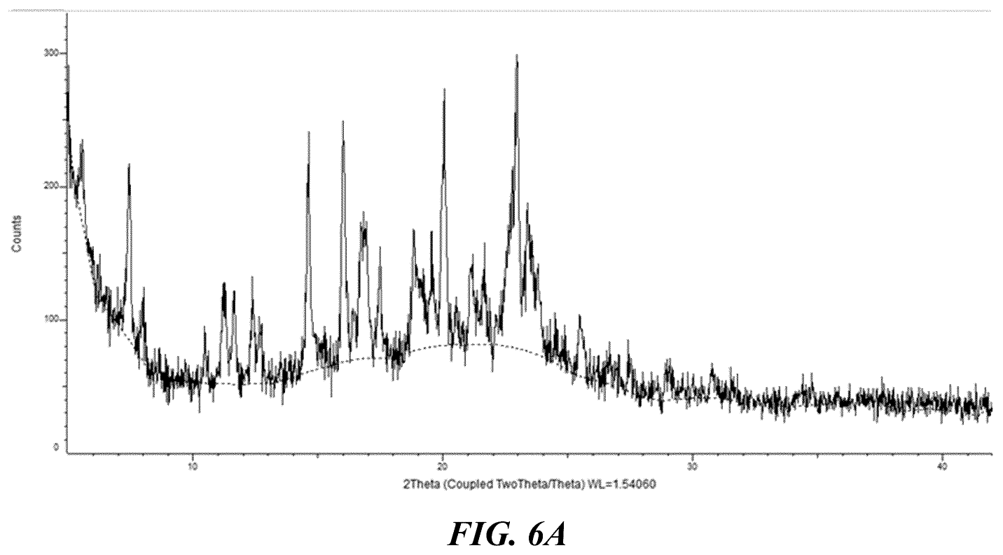

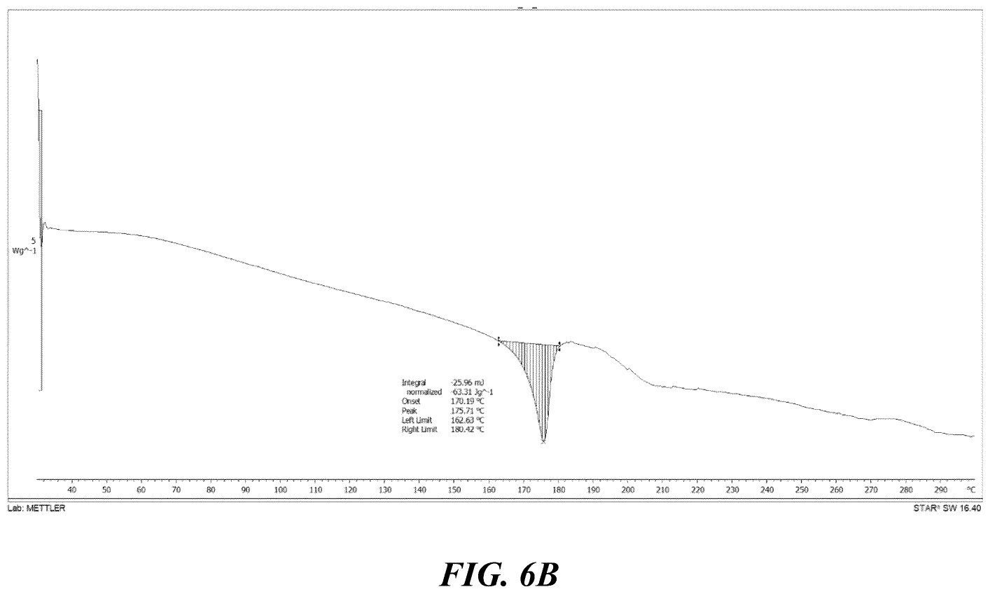

In embodiments, the L-tartrate salt of Compound 1 is crystalline. In embodiments, the L-tartrate salt of Compound 1 is characterized by an XRPD pattern substantially similar to FIG. 6A.

In embodiments, the L-tartrate salt of Compound 1 is characterized by a DSC thermogram comprising an endothermic onset at 170±5° C.

In embodiments, the L-tartrate salt of Compound 1 is characterized by a DSC thermogram substantially similar to FIG. 6B.

In embodiments, the L-tartrate salt of Compound 1 is characterized by an XRPD pattern having peaks at 16.0±0.2, 20.1±0.2, and 23.0±0.2 °2θ. In embodiments, the variance at any of these peaks is ±0.1 °2θ.

In embodiments, the L-tartrate salt of Compound 1 is characterized by an XRPD having peaks at 16.0±0.2, 20.1±0.2, and 23.0±0.2 °2θ and at least one (e.g., 1, 2, 3, 4, or 5) peak in an XRPD pattern selected from 7.5±0.2, 14.6±0.2, 19.6±0.2, 23.0±0.2 and 23.4±0.2 °2θ. In embodiments, the variance at any of these peaks is ±0.1 °2θ.

In embodiments, the L-tartrate salt of Compound 1 is characterized by an XRPD having peaks at 7.5±0.2, 14.6±0.2, 16.0±0.2, 19.6±0.2, 20.1±0.2, 23.0±0.2 and 23.4±0.2 °2θ. In embodiments, the variance at any of these peaks is ±0.1 °2θ.

In embodiments, the L-tartrate salt of Compound 1 is characterized by an XRPD pattern having one or more peaks at 5.5±0.2, 7.5±0.2, 8.0 0.2, 10.5±0.2, 11.3±0.2, 11.6±0.2, 12.4±0.2, 12.7±0.2, 14.6±0.2, 16.0±0.2, 16.5±0.2, 16.8±0.2, 17.5±0.2, 18.9±0.2, 19.6±0.2, 20.1±0.2, 20.6±0.2, 21.2±0.2, 21.7±0.2, 23.4±0.2, 23.0±0.2, 23.4±0.2, 23.8±0.2, 25.5±0.2, 27.5±0.2, 29.1±0.2, 30.8±0.2, and 34.8±0.2 °2θ. In embodiments, the variance at any of these peaks is ±0.1 °2θ. In embodiments, the L-tartrate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the L-tartrate salt of Compound 1 is characterized by an XRPD pattern comprising peaks shown in Table M.

TABLE M
XRPD Peak Positions for the L-tartrate Salt of Compound 1

| Peak position°2θ | Relative Intensity |
|---|---|
| 5.5 | 17.2% |
| 7.5 | 52.6% |
| 8.0 | 11.1% |
| 10.5 | 18.3% |
| 11.3 | 22.0% |
| 11.6 | 30.0% |
| 12.4 | 29.2% |
| 12.7 | 12.0% |
| 14.6 | 54.1% |
| 16.0 | 76.1% |
| 16.5 | 12.0% |
| 16.8 | 42.2% |
| 16.8 | 38.0% |
| 17.5 | 38.3% |
| 18.9 | 39.5% |
| 19.6 | 46.9% |
| 20.1 | 90.2% |
| 20.6 | 18.4% |
| 21.2 | 22.5% |
| 21.7 | 25.7% |
| 23.4 | 56.6% |
| 23.0 | 100.0% |
| 23.8 | 32.5% |
| 25.5 | 21.0% |
| 27.5 | 9.4% |
| 29.1 | 9.7% |
| 30.8 | 10.4% |
| 34.8 | 11.3% |

In embodiments, the L-tartrate salt of Compound 1 has a chemical purity of greater than about 80%, about 85%, about 90%, about 95%, about 96%, about 97%, about 98%, about 99%, 99.5%, or about 100% by weight as determined by HPLC.

In embodiments, the present disclosure provides a fumarate salt of Compound 1 having the following chemical formula:

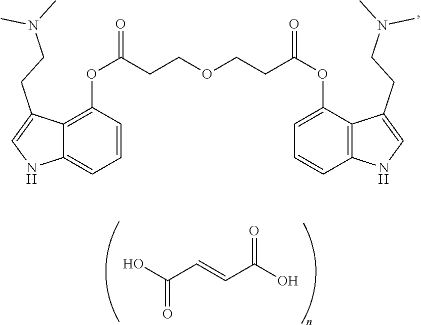

wherein n ranges from 1 to 3.

In embodiments, n is 1.

In embodiments, n is 1.5.

In embodiments, n is 2.

In embodiments, n is 2.5.

In embodiments, n is 3.

In embodiments, the fumarate salt of Compound 1 has the following chemical formula:

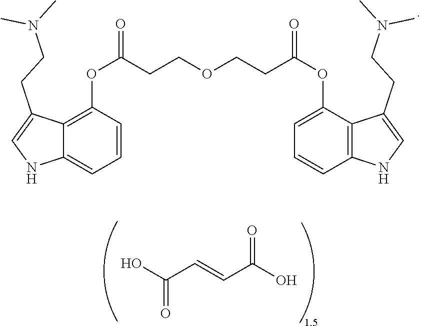

Such a fumarate salt is referred to herein as a sesquifumarate salt.

In embodiments, the fumarate salt of Compound 1 has the following chemical formula:

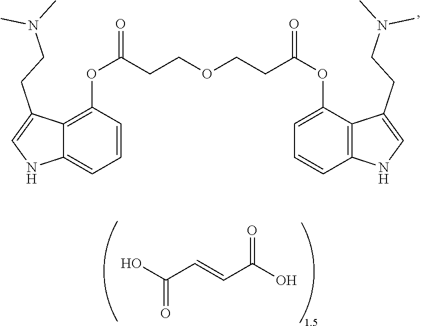

wherein the fumarate salt is characterized by an XRPD pattern having peaks at 8.7±0.2, 19.7±0.2, and 23.6±0.2 °2θ. In embodiments, the fumarate salt is further characterized by peaks in an XRPD pattern at 14.4±0.2, 21.8±0.2, and 22.2±0.2 °2θ.

In embodiments, the fumarate salt of Compound 1 has the following chemical formula:

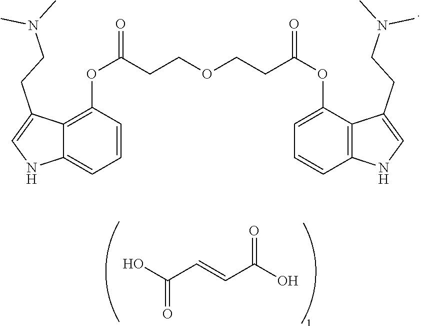

In embodiments, the fumarate salt of Compound 1 has the following chemical formula:

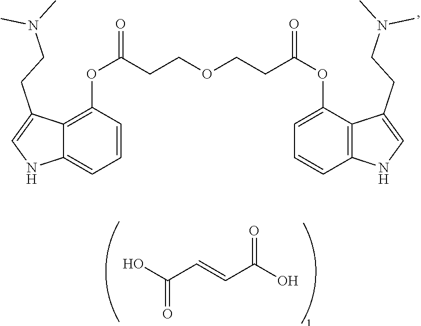

wherein characterized by an XRPD pattern having peaks at 12.8±0.2, 18.7±0.2, and 23.2±0.2 °2θ. In embodiments, the fumarate salt is further characterized by peaks in an XRPD pattern at 7.2±0.2, 10.4±0.2, 17.9±0.2, 19.3±0.2, and 21.4±0.2 °2θ.

In embodiments, the present disclosure provides the bis-formate salt of Compound 1.

In embodiments, the present disclosure provides a hydrochloride salt of Compound 1. In embodiments the hydrochloride salt of Compound 1 has the following formula:

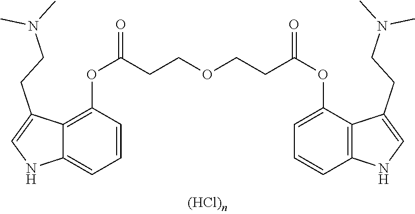

wherein n ranges from 1 to 2.

In embodiments, n is 1.

In embodiments, n is 1.5.

In embodiments, n is 2.

In embodiments, the hydrochloride salt of Compound 1 is crystalline.

In embodiments, the hydrochloride salt of Compound 1 is crystalline Form 10, Form 11, or mixtures thereof.

### Form 10:

In embodiments, the hydrochloride salt of Compound 1, is characterized by an XRPD pattern having peaks at 20.3±0.2, 20.7±0.2, and 23.3±0.2 °2θ. In embodiments, the variance at any of these peaks is ±0.1 °2θ.

In embodiments, the hydrochloride salt of Compound 1 is characterized by an XRPD having peaks at 20.3±0.2, 20.7±0.2, and 23.3±0.2 °2θ and at least one (e.g., 1, 2, 3, 4, or 5) peak in an XRPD pattern selected from 9.9±0.2, 16.1±0.2, 17.6±0.2, 19.7±0.2, and 26.7±0.2 °2θ. In embodiments, the variance at any of these peaks is ±0.1 °2θ.

In embodiments, the hydrochloride salt of Compound 1 is characterized by an XRPD having peaks at 9.9±0.2, 16.1±0.2, 17.6±0.2, 19.7±0.2, 20.3±0.2, 20.7±0.2, 23.3±0.2, and 26.7±0.2 °2θ. In embodiments, the variance at any of these peaks is ±0.1 °2θ.

In embodiments, the hydrochloride salt of Compound 1 is characterized by an XRPD pattern having one or more peaks at 7.5±0.2, 7.8±0.2, 9.9±0.2, 10.4±0.2, 11.2±0.2, 13.3±0.2, 13.7±0.2, 15.4±0.2, 16.1±0.2, 16.8±0.2, 17.6±0.2, 17.9±0.2, 18.2±0.2, 18.7±0.2, 19.7±0.2, 20.3±0.2, 20.7±0.2, 21.1±0.2, 21.4±0.2, 21.9±0.2, 22.4±0.2, 22.7±0.2, 23.3±0.2, 23.6±0.2, 23.9±0.2, 24.9±0.2, 25.2±0.2, 26.3±0.2, 26.7±0.2, 27.4±0.2, 28.2±0.2, 28.9±0.2, 29.7±0.2, 31.0±0.2, 31.5±0.2, 32.5±0.2, 33.0±0.2, 33.5±0.2, 33.8±0.2, 35.3±0.2, 36.2±0.2, 36.7±0.2, 37.1±0.2, 37.9±0.2, 38.8±0.2, 39.3±0.2, 40.0±0.2, and 40.8±0.2 °2θ. In embodiments, the variance at any of these peaks is ±0.1 °2θ. In embodiments, the hydrochloride salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

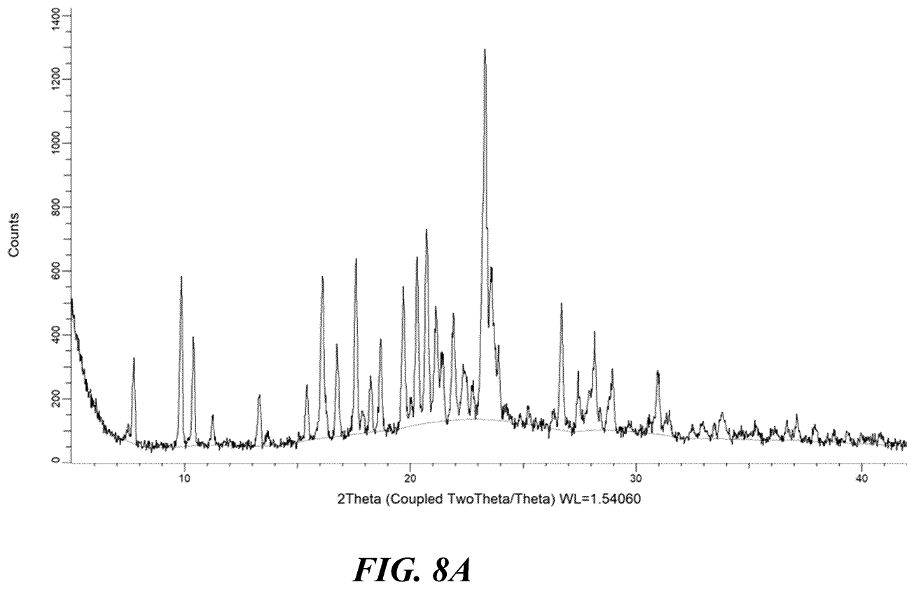

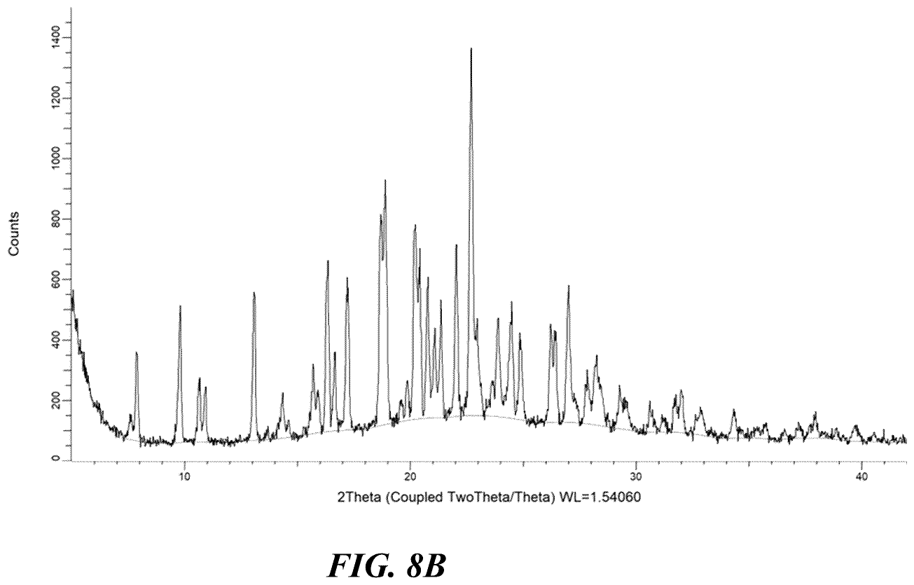

In embodiments, the hydrochloride salt of Compound 1 is characterized by an XRPD pattern substantially similar to FIG. 8A.

In embodiments, the hydrochloride salt of Compound 1 is characterized by an XRPD pattern comprising peaks shown in Table N.

TABLE N
XRPD Peak Positions for the Hydrochloride
Salt of Compound 1 (“Form 10”)

| Peak | Relative |
|---|---|
| position°2θ | Intensity |
| 7.5 | 2.9% |
| 7.8 | 21.6% |
| 9.9 | 42.7% |
| 10.4 | 26.1% |
| 11.2 | 6.5% |
| 13.3 | 12.5% |
| 13.7 | 3.4% |
| 15.4 | 14.8% |
| 16.1 | 44.0% |
| 16.8 | 25.1% |
| 17.6 | 44.6% |
| 17.9 | 5.6% |
| 18.2 | 15.1% |
| 18.7 | 24.8% |
| 19.7 | 37.9% |
| 20.3 | 47.3% |
| 20.7 | 51.2% |
| 21.1 | 29.1% |
| 21.4 | 14.6% |
| 21.9 | 26.7% |
| 22.4 | 11.0% |
| 22.7 | 8.8% |
| 23.3 | 100.0% |
| 23.6 | 35.7% |
| 23.9 | 22.6% |
| 24.9 | 3.4% |
| 25.2 | 5.7% |
| 26.3 | 4.7% |
| 26.7 | 36.5% |
| 27.4 | 18.1% |
| 28.2 | 28.1% |
| 28.9 | 18.8% |
| 29.7 | 2.1% |
| 31.0 | 18.7% |
| 31.5 | 8.3% |
| 32.5 | 4.6% |
| 33.0 | 4.2% |
| 33.5 | 5.2% |
| 33.8 | 7.6% |
| 35.3 | 4.3% |
| 36.2 | 4.0% |
| 36.7 | 6.5% |
| 37.1 | 6.7% |
| 37.9 | 4.0% |
| 38.8 | 4.0% |
| 39.3 | 4.7% |
| 40.0 | 3.1% |
| 40.8 | 2.7% |

In embodiments, the hydrochloride salt of Compound 1 is characterized by one, two, three, four, five, six, seven, eight, nine, ten, eleven, or more, XRPD peaks listed in Table N.

In embodiments, the hydrochloride salt of Compound 1 of Form 10 has a chemical purity of greater than about 80%, about 85%, about 90%, about 95%, about 96%, about 97%, about 98%, about 99%, 99.5%, or about 100% by weight as determined by HPLC.

In embodiments, the hydrochloride salt of Compound 1 is a hydrate.

### Form 11:

In embodiments, the hydrochloride salt of Compound 1, is characterized by an XRPD pattern having peaks at 20.2±0.2, 22.0±0.2, and 22.7±0.2 °2θ. In embodiments, the variance at any of these peaks is ±0.1 °2θ.

In embodiments, the hydrochloride salt of Compound 1 is characterized by an XRPD having peaks at 20.2±0.2, 22.00 0.2, and 22.7±0.2 °2θ and at least one (e.g., 1, 2, 3, 4, or 5) peak in an XRPD pattern selected from 13.1±0.2, 16.3±0.2, 18.8±0.2, 20.4±0.2, and 27.0±0.2 °2θ. In embodiments, the variance at any of these peaks is ±0.1 °2θ.

In embodiments, the hydrochloride salt of Compound 1 is characterized by an XRPD having peaks at 13.1±0.2, 16.3±0.2, 18.8±0.2, 20.2±0.2, 20.4±0.2, 22.0±0.2, 22.7±0.2 and 27.0 0.2, °2θ. In embodiments, the variance at any of these peaks is ±0.1 °2θ.

In embodiments, the hydrochloride salt of Compound 1 is characterized by an XRPD pattern having one or more peaks at 7.6±0.2, 7.9±0.2, 9.8±0.2, 10.7±0.2, 10.9±0.2, 13.1±0.2, 13.7±0.2, 14.3±0.2, 14.6±0.2, 15.7±0.2, 15.9±0.2, 16.3±0.2, 16.7±0.2, 17.2±0.2, 18.8±0.2, 19.6±0.2, 19.9±0.2, 20.2±0.2, 20.4±0.2, 20.8±0.2, 21.1±0.2, 21.4±0.2, 22.0±0.2, 22.7±0.2, 22.9±0.2, 23.9±0.2, 24.5±0.2, 24.9±0.2, 26.3±0.2, 27.0±0.2, 27.2±0.2, 27.8±0.2, 28.2±0.2, 28.4±0.2, 29.3±0.2, 29.5±0.2, 30.6±0.2, 31.7±0.2, 32.0±0.2, 32.9±0.2, 34.3±0.2, 35.7±0.2, 36.6±0.2, 37.2±0.2, 37.7±0.2, 37.9±0.2, 38.8±0.2, and 40.5±0.2 °2θ. In embodiments, the variance at any of these peaks is ±0.1 °2θ. In embodiments, the hydrochloride salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the hydrochloride salt of Compound 1 is characterized by an XRPD pattern substantially similar to FIG. 8B.

In embodiments, the hydrochloride salt of Compound 1 is characterized by an XRPD pattern comprising peaks shown in Table O.

TABLE O
XRPD Peak Positions for the Hydrochloride
Salt of Compound 1 (“Form 11”)

| Peak | Relative |
|---|---|
| position°2θ | Intensity |
| 7.6 | 5.2% |
| 7.9 | 21.3% |
| 9.8 | 34.8% |
| 10.7 | 13.6% |
| 10.9 | 13.8% |
| 13.1 | 37.7% |
| 13.7 | 3.4% |
| 14.3 | 12.1% |
| 14.6 | 4.7% |
| 15.7 | 19.3% |
| 15.9 | 8.7% |
| 16.3 | 46.0% |
| 16.7 | 17.9% |
| 17.2 | 34.2% |
| 18.8 | 45.0% |
| 19.6 | 5.0% |
| 19.9 | 10.1% |
| 20.2 | 47.3% |
| 20.4 | 37.8% |
| 20.8 | 35.7% |
| 21.1 | 22.7% |
| 21.4 | 33.1% |
| 22.0 | 47.2% |
| 22.7 | 100.0% |
| 22.9 | 24.0% |
| 23.9 | 25.6% |
| 24.5 | 29.5% |
| 24.9 | 19.8% |
| 26.3 | 10.8% |
| 27.0 | 37.0% |
| 27.2 | 6.4% |
| 27.8 | 13.2% |
| 28.2 | 18.2% |
| 28.4 | 9.5% |
| 29.3 | 4.4% |
| 29.5 | 8.8% |
| 30.6 | 10.2% |
| 31.7 | 6.5% |
| 32.0 | 12.3% |
| 32.9 | 7.0% |
| 34.3 | 5.1% |
| 35.7 | 4.0% |
| 36.6 | 2.2% |
| 37.2 | 4.2% |
| 37.7 | 3.6% |
| 37.9 | 5.8% |
| 38.8 | 3.6% |
| 40.5 | 3.2% |

In embodiments, the hydrochloride salt of Compound 1 is characterized by one, two, three, four, five, six, seven, eight, nine, ten, eleven, or more, XRPD peaks listed in Table O.

In embodiments, the hydrochloride salt of Compound 1 of Form 11 has a chemical purity of greater than about 80%, about 85%, about 90%, about 95%, about 96%, about 97%, about 98%, about 99%, 99.5%, or about 100% by weight as determined by HPLC.

In embodiments, the hydrochloride salt of Compound 1 is a hydrate.

In embodiments, the present disclosure provides a pyruvate salt of Compound 1. In embodiments, the pyruvate salt of Compound 1 has the following formula:

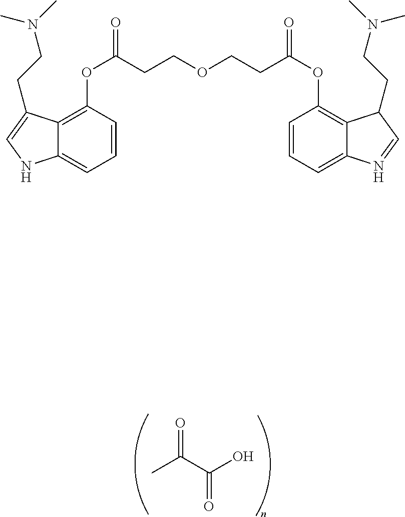

wherein n ranges from 1 to 2.

In embodiments, n is 1.

In embodiments, n is 1.5.

In embodiments, n is 2.

In embodiments, the pyruvate salt of Compound 1 is crystalline.

In embodiments, the pyruvate salt of Compound 1 is characterized by an XRPD pattern having peaks at 9.4±0.2, 18.8±0.2, and 22.0±0.2 °2θ.

In embodiments, the pyruvate salt of Compound 1 is characterized by an XRPD pattern having peaks at 9.4±0.2, 18.8±0.2, and 22.0±0.2 °2θ and at least one (e.g., 1, 2, or 3) peak in an XRPD pattern selected from 7.7±0.2, 13.9±0.2, 19.4±0.2, 21.1±0.2, and 22.7±0.2 °2θ. In embodiments, the variance at any of these peaks is ±0.1 °2θ.

In embodiments, the pyruvate salt of Compound 1 is characterized by an XRPD pattern having peaks at 7.7±0.2, 9.4±0.2, 13.9±0.2, 18.8±0.2, 19.4±0.2, 21.1 0.2, 22.0±0.2, and 22.7±0.2 °2θ. In embodiments, the variance at any of these peaks is ±0.1 °2θ.

In embodiments, the pyruvate salt of Compound 1 is characterized by an XRPD pattern having one or more peaks at 7.7±0.2, 8.0 0.2, 8.5±0.2, 9.4±0.2, 11.0±0.2, 11.3±0.2, 12.0±0.2, 12.9±0.2, 13.9±0.2, 14.9±0.2, 15.5±0.2, 15.9±0.2, 16.9±0.2, 17.2±0.2, 17.6±0.2, 18.4±0.2, 18.8±0.2, 19.4±0.2, 20.1±0.2, 20.6±0.2, 21.1±0.2, 21.5±0.2, 22.0±0.2, 22.7±0.2, 23.2±0.2, 23.6±0.2, 24.1±0.2, 24.3±0.2, 25.0±0.2, 25.6±0.2, 25.9±0.2, 26.4±0.2, 26.8±0.2, 28.0±0.2, 28.3±0.2, 28.8±0.2, 29.3±0.2, 29.7±0.2, 30.2±0.2, 30.7±0.2, 32.3±0.2, 32.6±0.2, 32.9±0.2, 33.5±0.2, 34.2±0.2, 34.6±0.2, 35.0±0.2, 35.5±0.2, 36.5±0.2, 37.0±0.2, 37.4±0.2, 39.0±0.2, and 39.7±0.2 °2θ. In embodiments, the pyruvate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, or 26 of the aforementioned peaks, or any range therein (e.g., from 1-26, 2-20, 3-15, or 3-10 of the aforementioned peaks).

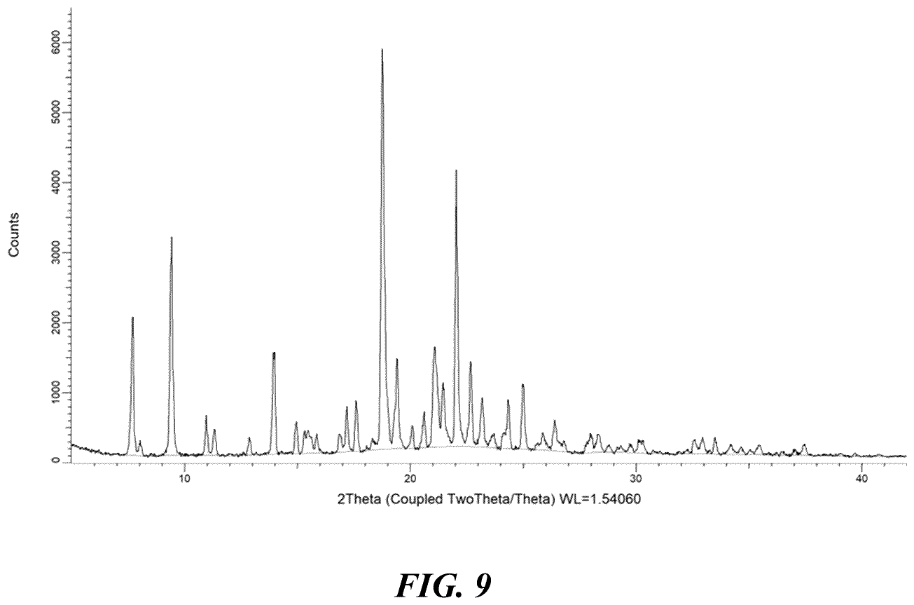

In embodiments, the pyruvate salt of Compound 1 is characterized by an XRPD pattern substantially similar to FIG. 9.

In embodiments, the pyruvate salt of Compound 1 is characterized by an XRPD pattern comprising peaks shown in Table P.

TABLE P
XRPD Peak Positions for the Pyruvate Salt of Compound 1

| Position°2θ | Relative Intensity |
|---|---|
| 7.7 | 32.9% |
| 8.0 | 3.8% |
| 8.5 | 0.7% |
| 9.4 | 53.7% |
| 11.0 | 9.9% |
| 11.3 | 6.3% |
| 12.0 | 0.6% |
| 12.9 | 4.5% |
| 13.9 | 24.5% |
| 14.9 | 7.4% |
| 15.5 | 5.5% |
| 15.9 | 5.0% |
| 16.9 | 4.1% |
| 17.2 | 11.8% |
| 17.6 | 12.5% |
| 18.4 | 1.5% |
| 18.8 | 100.0% |
| 19.4 | 23.7% |
| 20.1 | 6.0% |
| 20.6 | 9.0% |
| 21.1 | 24.0% |
| 21.5 | 15.7% |
| 22.0 | 76.5% |
| 22.7 | 23.8% |
| 23.2 | 12.5% |
| 23.6 | 3.2% |
| 24.3 | 13.2% |
| 25.0 | 16.5% |
| 25.6 | 1.9% |
| 25.9 | 4.5% |
| 26.4 | 8.5% |
| 26.8 | 2.8% |
| 28.0 | 4.7% |
| 28.3 | 5.0% |
| 28.8 | 1.8% |
| 29.3 | 1.4% |
| 29.7 | 1.5% |
| 30.2 | 2.1% |
| 30.7 | 1.1% |
| 32.3 | 1.1% |
| 32.6 | 2.9% |
| 32.9 | 4.3% |
| 33.5 | 5.1% |
| 34.2 | 2.0% |
| 34.6 | 2.0% |
| 35.0 | 1.3% |
| 35.5 | 2.3% |
| 36.5 | 1.3% |
| 37.0 | 0.7% |
| 37.4 | 2.7% |
| 39.0 | 1.0% |
| 39.7 | 1.0% |

In embodiments, pyruvate salt of Compound 1 is characterized by one, two, three, four, five, six, seven, eight, nine, ten, eleven, or more, XRPD peaks listed in Table P.

In embodiments, the pyruvate salt of Compound 1 has a chemical purity of greater than about 80%, about 85%, about 90%, about 95%, about 96%, about 97%, about 98%, about 99%, 99.5%, or about 100% by weight as determined by HPLC.

In embodiments, the pyruvate salt of Compound 1 is a hydrate.

In embodiments, the present disclosure provides a 3-oxodipropanoate salt of Compound 1. In embodiments the 3-oxodipropanoate salt of Compound 1 has the following formula:

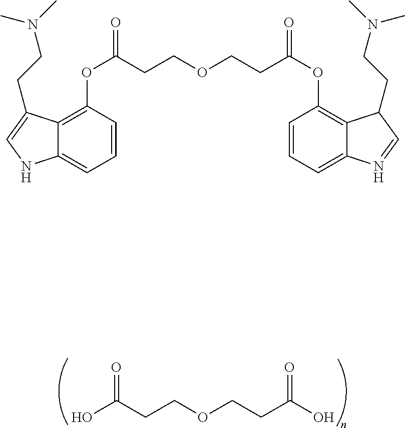

wherein n ranges from 1 to 2.

In embodiments, n is 1.

In embodiments, n is 1.5.

In embodiments, n is 2.

In embodiments, the 3-oxodipropanoate salt of Compound 1 is crystalline.

In embodiments, the 3-oxodipropanoate salt of Compound 1 is crystalline Form 12, Form 13, or mixtures thereof.

### Form 12:

In embodiments, the 3-oxodipropanoate salt of Compound 1, is characterized by an XRPD pattern having peaks at 18.0±0.2, 21.7±0.2, and 23.2±0.2 °2θ. In embodiments, the variance at any of these peaks is ±0.1 °2θ.

In embodiments, the 3-oxodipropanoate salt of Compound 1 is characterized by an XRPD having peaks at 18.0±0.2, 21.7±0.2, and 23.2±0.2 °2θ and at least one (e.g., 1, 2, 3, 4, or 5) peak in an XRPD pattern selected from 9.3±0.2, 10.8±0.2, 11.1±0.2, 16.4±0.2, and 19.3±0.2 °2θ. In embodiments, the variance at any of these peaks is ±0.1 °2θ.

In embodiments, the 3-oxodipropanoate salt of Compound 1 is characterized by an XRPD having peaks at 9.3±0.2, 10.8±0.2, 11.1±0.2, 16.4±0.2, 18.0±0.2, 19.3±0.2, 21.7±0.2, and 23.2±0.2 °2θ. In embodiments, the variance at any of these peaks is ±0.1 °2θ. In embodiments, the 3-oxodipropanoate salt of Compound 1 is characterized by an XRPD pattern having one or more peaks at 7.2±0.2, 7.7±0.2, 9.3±0.2, 10.8±0.2, 11.1±0.2, 13.0 0.2, 14.0±0.2, 14.6±0.2, 14.8±0.2, 15.4±0.2, 16.4±0.2, 16.6±0.2, 16.7±0.2, 18.0±0.2, 18.5±0.2, 18.7±0.2, 19.3±0.2, 20.0±0.2, 21.0±0.2, 21.1±0.2, 21.4±0.2, 21.7±0.2, 22.0±0.2, 22.5±0.2, 23.2±0.2, 23.5±0.2, 23.7±0.2, 24.7±0.2, 24.8±0.2, 25.8±0.2, 26.2±0.2, 28.1±0.2, 28.4±0.2, 29.8±0.2, 30.3±0.2, 30.5±0.2, 32.6±0.2, 33.1±0.2, 33.4±0.2, 33.7±0.2, 34.4±0.2, and 37.9±0.2 °2θ. In embodiments, the variance at any of these peaks is ±0.1 °2θ. In embodiments, the 3-oxodipropanoate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

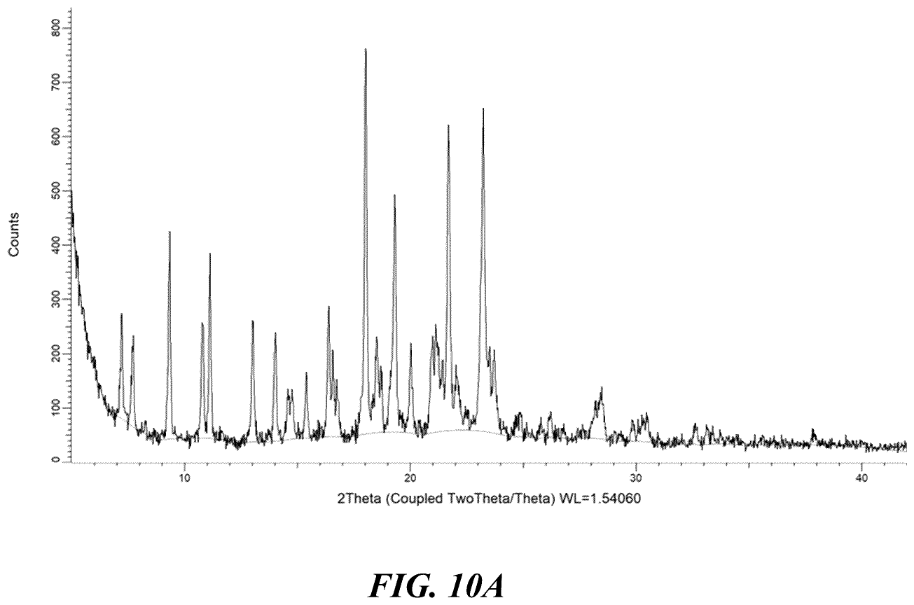

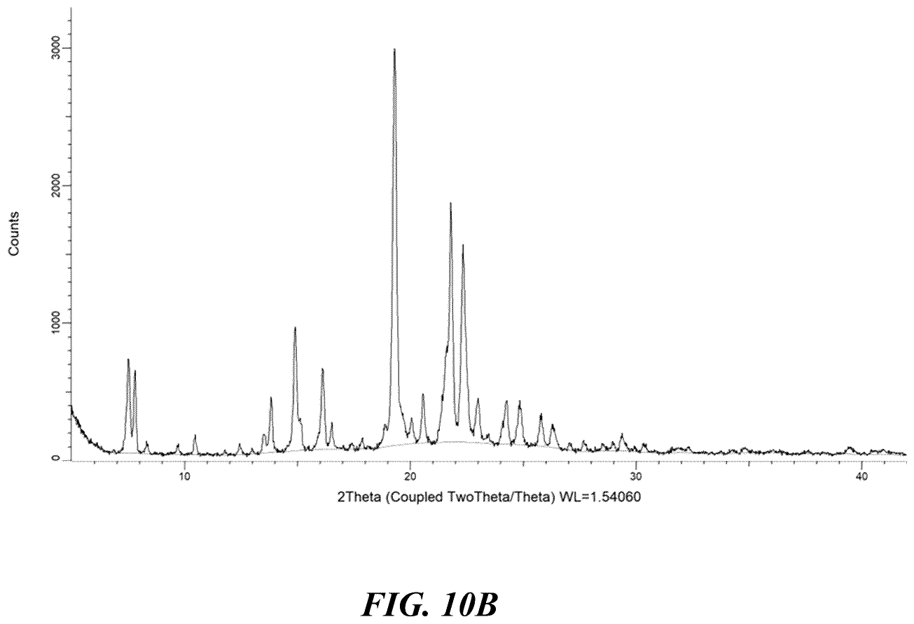

In embodiments, the 3-oxodipropanoate salt of Compound 1 is characterized by an XRPD pattern substantially similar to FIG. 10A.

In embodiments, the 3-oxodipropanoate salt of Compound 1 is characterized by an XRPD pattern comprising peaks shown in Table Q.

TABLE Q
XRPD Peak Positions for the 3-Oxodipropanoate
Salt of Compound 1 (“Form 12”)

| Peak | Relative |
|---|---|
| position°2θ | Intensity |
| 7.2 | 24.8% |
| 7.7 | 17.9% |
| 9.3 | 50.0% |
| 10.8 | 28.5% |
| 11.1 | 45.2% |
| 13.0 | 28.2% |
| 14.0 | 25.1% |
| 14.6 | 10.5% |
| 14.8 | 13.4% |
| 15.4 | 17.9% |
| 16.4 | 31.0% |
| 16.6 | 24.9% |
| 16.7 | 14.9% |
| 18.0 | 100.0% |
| 18.5 | 23.9% |
| 18.7 | 14.2% |
| 19.3 | 61.1% |
| 20.0 | 24.2% |
| 21.0 | 23.0% |
| 21.1 | 26.2% |
| 21.4 | 18.8% |
| 21.7 | 77.4% |
| 22.0 | 13.1% |
| 22.5 | 3.0% |
| 23.2 | 89.8% |
| 23.5 | 21.5% |
| 23.7 | 20.5% |
| 24.7 | 4.3% |
| 24.8 | 6.5% |
| 25.8 | 6.7% |
| 26.2 | 6.1% |
| 28.1 | 6.0% |
| 28.4 | 9.6% |
| 29.8 | 5.2% |
| 30.3 | 4.4% |
| 30.5 | 6.4% |
| 32.6 | 6.1% |
| 33.1 | 4.0% |
| 33.4 | 4.7% |
| 33.7 | 3.5% |
| 34.4 | 3.5% |
| 37.9 | 3.5% |

In embodiments, the 3-oxodipropanoate salt of Compound 1 is characterized by one, two, three, four, five, six, seven, eight, nine, ten, eleven, or more, XRPD peaks listed in Table 0.

In embodiments, the 3-oxodipropanoate salt of Compound 1 of Form 12 has a chemical purity of greater than about 80%, about 85%, about 90%, about 95%, about 96%, about 97%, about 98%, about 99%, 99.5%, or about 100% by weight as determined by HPLC.

In embodiments, the 3-oxodipropanoate salt of Compound 1 is a hydrate.

### Form 13:

In embodiments, the 3-oxodipropanoate salt of Compound 1, is characterized by an XRPD pattern having peaks at 19.3±0.2, 21.8±0.2, and 22.3±0.2 °2θ. In embodiments, the variance at any of these peaks is ±0.1 °2θ.

In embodiments, the 3-oxodipropanoate salt of Compound 1 is characterized by an XRPD having peaks at 19.3±0.2, 21.8±0.2, and 22.3±0.2 °2θ and at least one (e.g., 1, 2, 3, 4, or 5) peak in an XRPD pattern selected from 7.5±0.2, 7.8±0.2, 13.8±0.2, 14.9±0.2, and 16.1±0.2 °2θ. In embodiments, the variance at any of these peaks is ±0.1 °2θ.

In embodiments, the 3-oxodipropanoate salt of Compound 1 is characterized by an XRPD having peaks at 7.5±0.2, 7.8±0.2, 13.8±0.2, 14.9±0.2, 16.1±0.2, 19.3±0.2, 21.8±0.2, and 22.3±0.2 °2θ. In embodiments, the variance at any of these peaks is ±0.1 °2θ.

In embodiments, the 3-oxodipropanoate salt of Compound 1 is characterized by an XRPD pattern having one or more peaks at 7.5±0.2, 7.8±0.2, 8.3±0.2, 9.7±0.2, 10.5±0.2, 11.8±0.2, 12.4±0.2, 13.0±0.2, 13.5±0.2, 13.8±0.2, 14.9±0.2, 16.1±0.2, 16.5±0.2, 17.4±0.2, 17.9±0.2, 18.9±0.2, 19.3±0.2, 20.1±0.2, 20.6±0.2, 21.8±0.2, 22.3±0.2, 23.0±0.2, 23.4±0.2, 24.3±0.2, 24.8±0.2, 25.8±0.2, 26.3±0.2, 27.0±0.2, 27.7±0.2, 28.5±0.2, 29.0±0.2, 29.4±0.2, 30.3±0.2, 32.3±0.2, 34.2±0.2, 34.8±0.2, and 39.4±0.2 °2θ. In embodiments, the variance at any of these peaks is ±0.1 °2θ. In embodiments, the 3-oxodipropanoate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the 3-oxodipropanoate salt of Compound 1 is characterized by an XRPD pattern substantially similar to FIG. 10B.

In embodiments, the 3-oxodipropanoate salt of Compound 1 is characterized by an XRPD pattern comprising peaks shown in Table R.

TABLE R
XRPD Peak Positions for the 3-Oxodipropanoate
Salt of Compound 1 (“Form 13”)

| Peak | Relative |
|---|---|
| position°2θ | Intensity |
| 7.5 | 23.1% |
| 7.8 | 20.6% |
| 8.3 | 1.7% |
| 9.7 | 1.6% |
| 10.5 | 4.6% |
| 11.8 | 1.5% |
| 12.4 | 2.8% |
| 13.0 | 1.9% |
| 13.5 | 4.5% |
| 13.8 | 14.1% |
| 14.9 | 31.4% |
| 16.1 | 21.1% |
| 16.5 | 7.7% |
| 17.4 | 1.7% |
| 17.9 | 3.0% |
| 18.9 | 5.8% |
| 19.3 | 100.0% |
| 20.1 | 7.1% |
| 20.6 | 13.5% |
| 21.8 | 62.9% |
| 22.3 | 50.7% |
| 23.0 | 11.4% |
| 23.4 | 2.4% |
| 24.3 | 11.3% |
| 24.8 | 11.5% |
| 25.8 | 8.3% |
| 26.3 | 5.7% |
| 27.0 | 2.3% |
| 27.7 | 3.1% |
| 28.5 | 1.4% |
| 29.0 | 3.2% |
| 29.4 | 4.3% |
| 30.3 | 2.4% |
| 32.3 | 2.1% |
| 34.2 | 1.2% |
| 34.8 | 1.1% |
| 39.4 | 1.8% |

In embodiments, 3-oxodipropanoate salt of Compound 1 is characterized by one, two, three, four, five, six, seven, eight, nine, ten, eleven, or more, XRPD peaks listed in Table R.

In embodiments, the 3-oxodipropanoate salt of Compound 1 of Form 13 has a chemical purity of greater than about 80%, about 85%, about 90%, about 95%, about 96%, about 97%, about 98%, about 99%, 99.5%, or about 100% by weight as determined by HPLC.

In embodiments, the 3-oxodipropanoate salt of Compound 1 is a hydrate.

In embodiments, the present disclosure provides an adipate salt of Compound 1. In embodiments, the adipate salt of Compound 1 has the following formula:

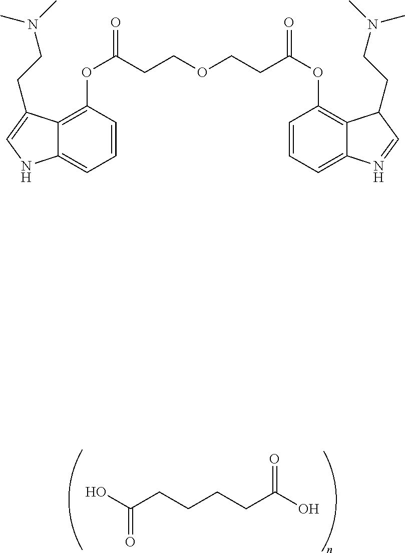

wherein n ranges from 1 to 3.

In embodiments, n is 1.

In embodiments, n is 1.5.

In embodiments, n is 2.

In embodiments, n is 2.5.

In embodiments, n is 3.

In embodiments, the adipate salt of Compound 1 is crystalline.

In embodiments, the adipate salt of Compound 1 is crystalline Form 14, Form 15, Form 16, or mixtures thereof.

### Form 14:

In embodiments, the adipate salt of Compound 1, is characterized by an XRPD pattern having peaks at 7.5±0.2, 18.00 0.2, and 18.5±0.2 °2θ. In embodiments, the variance at any of these peaks is ±0.1 °2θ.

In embodiments, the adipate salt of Compound 1 is characterized by an XRPD having peaks at 7.5±0.2, 18.0±0.2, and 18.5±0.2 °2θ and at least one (e.g., 1, 2, 3, 4, or 5) peak in an XRPD pattern selected from 9.3±0.2, 14.2±0.2, 15.3±0.2, 21.2±0.2, and 22.3±0.2 °2θ. In embodiments, the variance at any of these peaks is ±0.1 °2θ.

In embodiments, the adipate salt of Compound 1 is characterized by an XRPD having peaks at 7.5±0.2, 9.3±0.2, 14.2±0.2, 15.3±0.2, 18.0±0.2, 18.5±0.2, 21.2±0.2, and 22.3±0.2 °2θ. In embodiments, the variance at any of these peaks is ±0.1 °2θ.

In embodiments, the adipate salt of Compound 1 is characterized by an XRPD pattern having one or more peaks at 7.5±0.2, 8.6±0.2, 9.3±0.2, 11.3±0.2, 12.6±0.2, 14.2±0.2, 15.0±0.2, 15.3±0.2, 16.3±0.2, 16.7±0.2, 17.1±0.2, 18.0±0.2, 18.5±0.2, 19.3±0.2, 20.8±0.2, 21.2±0.2, 21.7±0.2, 22.3±0.2, 22.8±0.2, 23.4±0.2, 23.8±0.2, 23.9±0.2, 24.3±0.2, 25.1±0.2, 25.8±0.2, 27.0±0.2, 28.1±0.2, 28.6±0.2, 29.8±0.2, 30.8±0.2, 33.7±0.2, 34.8±0.2, 35.5±0.2, 37.4±0.2, and 40.1±0.2 °2θ. In embodiments, the variance at any of these peaks is ±0.1 °2θ. In embodiments, the adipate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

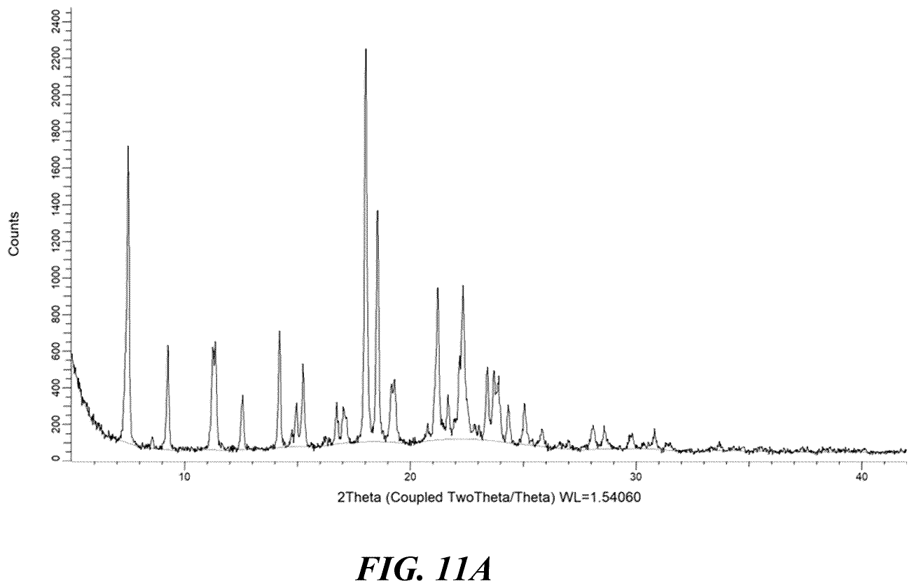

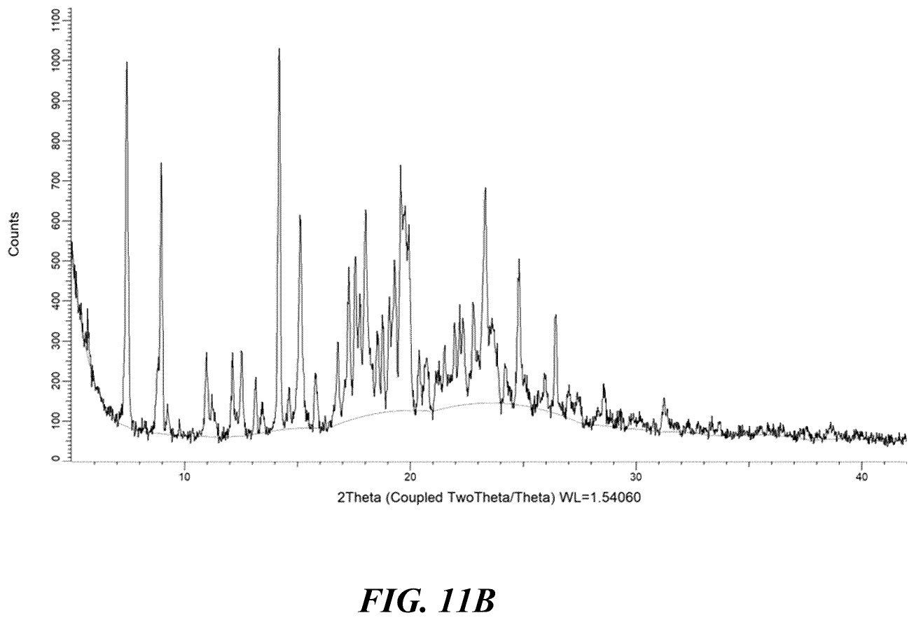

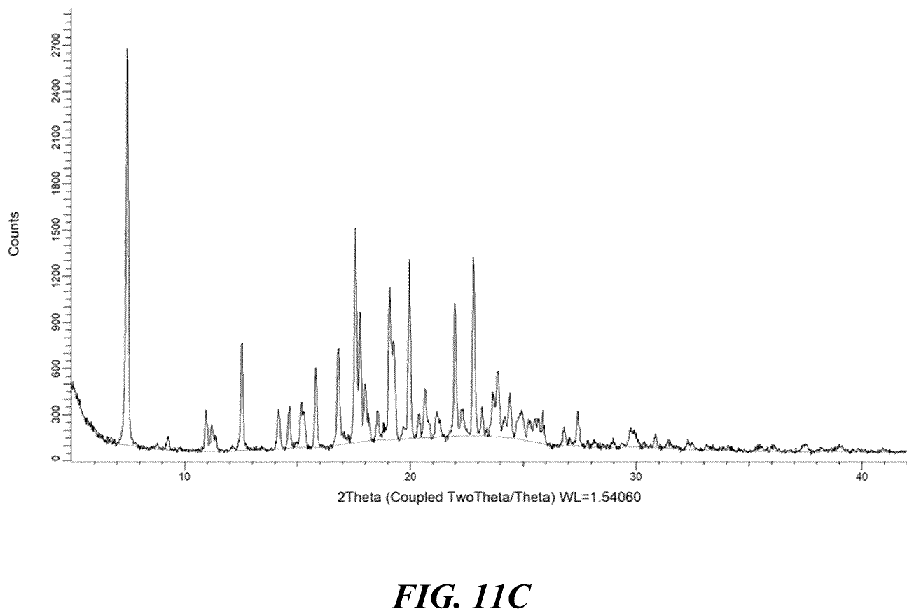

In embodiments, the adipate salt of Compound 1 is characterized by an XRPD pattern substantially similar to FIG. 11A.

In embodiments, the adipate salt of Compound 1 is characterized by an XRPD pattern comprising peaks shown in Table S.

TABLE S
XRPD Peak Positions for the Adipate Salt
of Compound 1 (“Form 14”)

| Peak | Relative |
|---|---|
| position°2θ | Intensity |
| 7.5 | 71.5% |
| 8.6 | 2.6% |
| 9.3 | 26.1% |
| 11.3 | 19.4% |
| 12.6 | 13.0% |
| 14.2 | 27.8% |
| 15.0 | 10.5% |
| 15.3 | 21.1% |
| 16.3 | 3.1% |
| 16.7 | 11.3% |
| 17.1 | 8.1% |
| 18.0 | 100.0% |
| 18.5 | 62.2% |
| 19.3 | 16.0% |
| 20.8 | 5.1% |
| 21.2 | 40.2% |
| 21.7 | 12.4% |
| 22.3 | 37.8% |
| 22.8 | 4.3% |
| 23.4 | 18.8% |
| 23.8 | 7.8% |
| 23.9 | 14.4% |
| 24.3 | 9.3% |
| 25.1 | 11.7% |
| 25.8 | 4.8% |
| 27.0 | 1.9% |
| 28.1 | 5.8% |
| 28.6 | 5.5% |
| 29.8 | 3.1% |
| 30.8 | 5.6% |
| 33.7 | 2.6% |
| 34.8 | 1.1% |
| 35.5 | 1.0% |
| 37.4 | 1.4% |
| 40.1 | 1.3% |

In embodiments, the adipate salt of Compound 1 is characterized by one, two, three, four, five, six, seven, eight, nine, ten, eleven, or more, XRPD peaks listed in Table S.

In embodiments, the adipate salt of Compound 1 of Form 14 has a chemical purity of greater than about 80%, about 85%, about 90%, about 95%, about 96%, about 97%, about 98%, about 99%, 99.5%, or about 100% by weight as determined by HPLC.

In embodiments, the adipate salt of Compound 1 is a hydrate.

### Form 15:

In embodiments, the adipate salt of Compound 1, is characterized by an XRPD pattern having peaks at 7.4±0.2, 9.00 0.2, and 14.2±0.2 °2θ. In embodiments, the variance at any of these peaks is ±0.1 °2θ.

In embodiments, the adipate salt of Compound 1 is characterized by an XRPD having peaks at 7.4±0.2, 9.00 0.2, and 14.2±0.2 °2θ and at least one (e.g., 1, 2, 3, 4, or 5) peak in an XRPD pattern selected from 15.1±0.2, 18.0±0.2, 19.7±0.2, 19.9±0.2, and 23.3±0.2 °2θ. In embodiments, the variance at any of these peaks is ±0.1 °2θ.

In embodiments, the adipate salt of Compound 1 is characterized by an XRPD having peaks at 7.4±0.2, 9.0±0.2, 14.2±0.2, 15.1±0.2, 18.0±0.2, 19.7±0.2, 19.9±0.2, and 23.3±0.2 °2θ. In embodiments, the variance at any of these peaks is ±0.1 °2θ.

In embodiments, the adipate salt of Compound 1 is characterized by an XRPD pattern having one or more peaks at 7.4±0.2, 9.0 0.2, 9.3±0.2, 9.8±0.2, 11.0±0.2, 11.2±0.2, 12.1±0.2, 12.5±0.2, 13.1±0.2, 13.4±0.2, 14.2±0.2, 14.6±0.2, 15.1±0.2, 15.8±0.2, 16.8±0.2, 17.3±0.2, 17.6±0.2, 18.0±0.2, 18.5±0.2, 18.8±0.2, 19.1±0.2, 19.3±0.2, 19.7±0.2, 19.9±0.2, 20.4±0.2, 20.7±0.2, 21.2±0.2, 21.5±0.2, 22.0±0.2, 22.3±0.2, 22.8±0.2, 23.3±0.2, 23.6±0.2, 24.2±0.2, 24.8±0.2, 25.0±0.2, 26.0±0.2, 26.4±0.2, 27.0±0.2, 27.4±0.2, 28.6±0.2, 29.9±0.2, 31.2±0.2, 32.3±0.2, 32.7±0.2, 33.4±0.2, 33.7±0.2, 37.5±0.2, and 38.6±0.2 °2θ. In embodiments, the variance at any of these peaks is ±0.1 °2θ. In embodiments, the adipate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the adipate salt of Compound 1 is characterized by an XRPD pattern substantially similar to FIG. 11B.

In embodiments, the adipate salt of Compound 1 is characterized by an XRPD pattern comprising peaks shown in Table T.

TABLE T
XRPD Peak Positions for the Adipate Salt
of Compound 1 (“Form 15”)

| Peak | Relative |
|---|---|
| position°2θ | Intensity |
| 7.4 | 91.1% |
| 9.0 | 67.3% |
| 9.3 | 6.9% |
| 9.8 | 4.9% |
| 11.0 | 21.3% |
| 11.2 | 8.2% |
| 12.1 | 20.3% |
| 12.5 | 23.2% |
| 13.1 | 13.9% |
| 13.4 | 5.8% |
| 14.2 | 100.0% |
| 14.6 | 11.5% |
| 15.1 | 51.1% |
| 15.8 | 13.2% |
| 16.8 | 23.8% |
| 17.3 | 40.1% |
| 17.6 | 19.6% |
| 18.0 | 54.4% |
| 18.5 | 17.3% |
| 18.8 | 23.3% |
| 19.1 | 31.3% |
| 19.3 | 40.6% |
| 19.7 | 48.4% |
| 19.9 | 48.6% |
| 20.4 | 15.8% |
| 20.7 | 14.0% |
| 21.2 | 8.9% |
| 21.5 | 16.7% |
| 22.0 | 23.9% |
| 22.3 | 23.0% |
| 22.8 | 30.9% |
| 23.3 | 59.2% |
| 23.6 | 21.8% |
| 24.2 | 9.9% |
| 24.8 | 35.3% |
| 25.0 | 6.4% |
| 26.0 | 9.0% |
| 26.4 | 30.2% |
| 27.0 | 9.2% |
| 27.4 | 5.8% |
| 28.6 | 9.1% |
| 29.9 | 3.3% |
| 31.2 | 10.0% |
| 32.3 | 4.4% |
| 32.7 | 2.9% |
| 33.4 | 4.1% |
| 33.7 | 3.9% |
| 37.5 | 4.4% |
| 38.6 | 3.1% |

In embodiments, the adipate salt of Compound 1 is characterized by one, two, three, four, five, six, seven, eight, nine, ten, eleven, or more, XRPD peaks listed in Table T.

In embodiments, the adipate salt of Compound 1 of Form 15 has a chemical purity of greater than about 80%, about 85%, about 90%, about 95%, about 96%, about 97%, about 98%, about 99%, 99.5%, or about 100% by weight as determined by HPLC.

In embodiments, the adipate salt of Compound 1 is a hydrate.

### Form 16:

In embodiments, the adipate salt of Compound 1, is characterized by an XRPD pattern having peaks at 7.5±0.2, 17.6±0.2, and 22.8±0.2 °2θ. In embodiments, the variance at any of these peaks is ±0.1 °2θ.

In embodiments, the adipate salt of Compound 1 is characterized by an XRPD having peaks at 7.5±0.2, 17.6±0.2, and 22.8±0.2 °2θ and at least one (e.g., 1, 2, 3, 4, or 5) peak in an XRPD pattern selected from 12.5±0.2, 16.8±0.2, 17.8±0.2, 20.0 0.2, and 22.0±0.2 °2θ. In embodiments, the variance at any of these peaks is ±0.1 °2θ.

In embodiments, the adipate salt of Compound 1 is characterized by an XRPD having peaks at 7.5±0.2, 12.5±0.2, 16.8±0.2, 17.6±0.2, 17.8±0.2, 20.0 0.2, 22.0±0.2, and 22.8±0.2 °2θ. In embodiments, the variance at any of these peaks is ±0.1 °2θ.

In embodiments, the adipate salt of Compound 1 is characterized by an XRPD pattern having one or more peaks at 7.5±0.2, 9.3±0.2, 11.0±0.2, 11.2±0.2, 12.1±0.2, 12.5±0.2, 14.2±0.2, 14.6±0.2, 15.2±0.2, 15.8±0.2, 16.8±0.2, 17.6±0.2, 17.8±0.2, 18.0±0.2, 18.6±0.2, 19.2±0.2, 20.0±0.2, 20.4±0.2, 20.7±0.2, 21.2±0.2, 22.0±0.2, 22.3±0.2, 22.8±0.2, 23.2±0.2, 23.7±0.2, 23.9±0.2, 24.4±0.2, 24.9±0.2, 25.3±0.2, 25.6±0.2, 25.9±0.2, 26.8±0.2, 27.4±0.2, 29.0±0.2, 29.8±0.2, 30.9±0.2, 31.4±0.2, 32.4±0.2, 35.5±0.2, 36.1±0.2, 37.5±0.2, and 39.0±0.2 °2θ. In embodiments, the variance at any of these peaks is ±0.1 °2θ. In embodiments, the adipate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, 26, 27, 28, 29, 30, 31, 32, 33, 34, 35, 36, or 37 of the aforementioned peaks, or any range therein (e.g., from 1-37, 1-30, 2-20, 3-15, or 3-10 of the aforementioned peaks).

In embodiments, the adipate salt of Compound 1 is characterized by an XRPD pattern substantially similar to FIG. 11C.

In embodiments, the adipate salt of Compound 1 is characterized by an XRPD pattern comprising peaks shown in Table U.

TABLE U
XRPD Peak Positions for the Adipate Salt
of Compound 1 (“Form 16”)

| Peak | Relative |
|---|---|
| position°2θ | Intensity |
| 7.5 | 100.0% |
| 9.3 | 2.6% |
| 11.0 | 10.4% |
| 11.2 | 6.8% |
| 12.1 | 1.7% |
| 12.5 | 28.3% |
| 14.2 | 10.6% |
| 14.6 | 9.8% |
| 15.2 | 6.7% |
| 15.8 | 20.5% |
| 16.8 | 25.5% |
| 17.6 | 58.6% |
| 17.8 | 36.2% |
| 18.0 | 14.6% |
| 18.6 | 8.0% |
| 19.2 | 20.1% |
| 20.0 | 49.2% |
| 20.4 | 6.8% |
| 20.7 | 13.5% |
| 21.2 | 6.6% |
| 22.0 | 38.7% |
| 22.3 | 7.7% |
| 22.8 | 50.5% |
| 23.2 | 8.3% |
| 23.7 | 11.4% |
| 23.9 | 17.8% |
| 24.4 | 11.6% |
| 24.9 | 7.7% |
| 25.3 | 4.5% |
| 25.6 | 2.9% |
| 25.9 | 10.5% |
| 26.8 | 4.9% |
| 27.4 | 11.3% |
| 29.0 | 3.1% |
| 29.8 | 3.0% |
| 30.9 | 4.2% |
| 31.4 | 1.9% |
| 32.4 | 1.1% |
| 35.5 | 1.8% |
| 36.1 | 1.6% |
| 37.5 | 2.0% |
| 39.0 | 1.7% |

In embodiments, the adipate salt of Compound 1 is characterized by one, two, three, four, five, six, seven, eight, nine, ten, eleven, or more, XRPD peaks listed in Table U.

In embodiments, the adipate salt of Compound 1 of Form 16 has a chemical purity of greater than about 80%, about 85%, about 90%, about 95%, about 96%, about 97%, about 98%, about 99%, 99.5%, or about 100% by weight as determined by HPLC.

In embodiments, the adipate salt of Compound 1 is a hydrate.

In embodiments, the present provides an oxalate salt of Compound 1. In embodiments the oxalate salt of Compound 1 has the following formula:

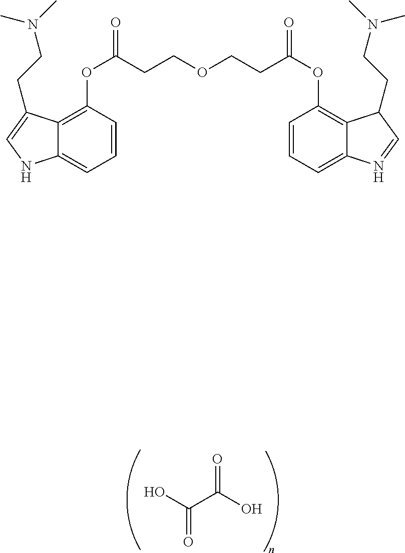

wherein n ranges from 1 to 2.

In embodiments, n is 1.

In embodiments, n is 1.5.

In embodiments, n is 2.

In embodiments, the oxalate salt of Compound 1 is crystalline.

In embodiments, the oxalate salt of Compound 1 is characterized by an XRPD pattern having peaks at 13.9±0.2, 21.9±0.2, and 24.8±0.2 °2θ.

In embodiments, the oxalate salt of Compound 1 is characterized by an XRPD pattern having peaks at 13.9±0.2, 21.9±0.2, and 24.8±0.2 °2θ and at least one (e.g., 1, 2, or 3) peak in an XRPD pattern selected from 13.4±0.2, 14.3±0.2, 17.1±0.2, 19.4±0.2, and 27.6±0.2 °2θ. In embodiments, the variance at any of these peaks is ±0.1 °2θ.

In embodiments, the oxalate salt of Compound 1 is characterized by an XRPD pattern having peaks at 13.4±0.2, 13.9±0.2, 14.3±0.2, 17.1±0.2, 19.4±0.2, 21.9±0.2, 24.8±0.2, and 27.6±0.2 °2θ. In embodiments, the variance at any of these peaks is ±0.1 °2θ.

In embodiments, the oxalate salt of Compound 1 is characterized by an XRPD pattern having one or more peaks at 8.9±0.2, 11.6±0.2, 13.4±0.2, 13.9±0.2, 14.3±0.2, 16.3±0.2, 17.1±0.2, 18.1±0.2, 19.4±0.2, 19.3±0.2, 20.2±0.2, 21.5±0.2, 21.9±0.2, 23.1±0.2, 24.1±0.2, 24.8±0.2, 25.0±0.2, 26.7±0.2, 27.6±0.2, 34.4±0.2, and 34.8±0.2 °2θ. In embodiments, the oxalate salt of Compound 1 may be characterized by 1, 2, 3, 4, 5, 6, 7, 8, 9, 10, 11, 12, 13, 14, 15, 16, 16, 17, 18, 19, 20, 21, 22, 23, 24, 25, or 26 of the aforementioned peaks, or any range therein (e.g., from 1-26, 2-20, 3-15, or 3-10 of the aforementioned peaks).

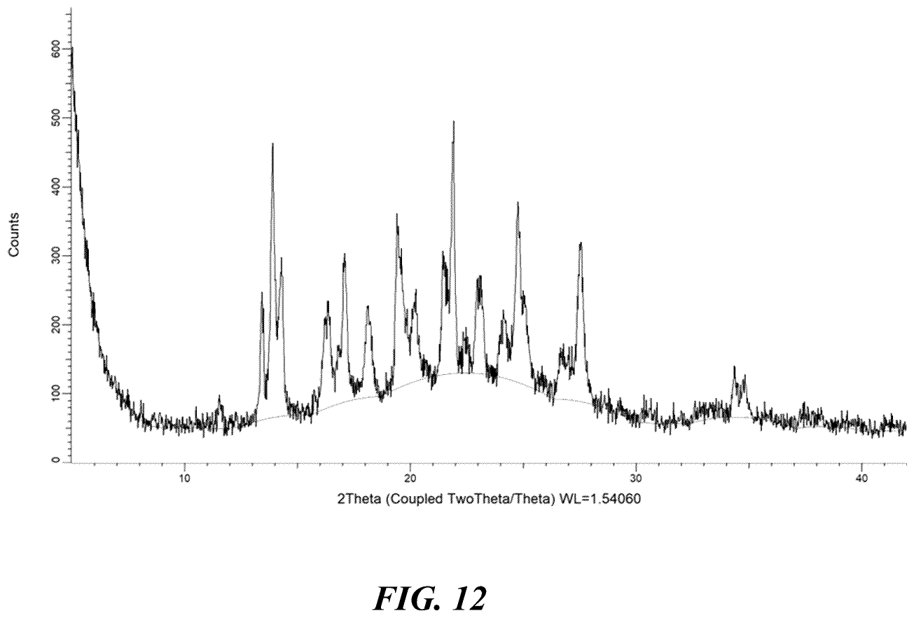

In embodiments, the oxalate salt of Compound 1 is characterized by an XRPD pattern substantially similar to FIG. 12.

In embodiments, the oxalate salt of Compound 1 is characterized by an XRPD pattern comprising peaks shown in Table V.

TABLE V
XRPD Peak Positions for the Oxalate Salt of Compound 1

| Position°2θ | Relative Intensity |
|---|---|
| 8.9 | 4.7% |
| 11.6 | 12.3% |
| 13.4 | 44.4% |
| 13.9 | 100.0% |
| 14.3 | 56.7% |
| 16.3 | 40.8% |
| 17.1 | 51.4% |
| 18.1 | 33.5% |
| 19.4 | 56.1% |
| 19.3 | 17.1% |
| 20.2 | 28.5% |
| 21.5 | 33.0% |
| 21.9 | 93.6% |
| 23.1 | 28.3% |
| 24.1 | 28.1% |
| 24.8 | 69.7% |
| 25.0 | 34.4% |
| 26.7 | 13.6% |
| 27.6 | 57.0% |
| 34.4 | 16.4% |
| 34.8 | 11.8% |

In embodiments, the oxalate salt of Compound 1 is characterized by one, two, three, four, five, six, seven, eight, nine, ten, eleven, or more, XRPD peaks listed in Table V.

In embodiments, the oxalate salt of Compound 1 has a chemical purity of greater than about 80%, about 85%, about 90%, about 95%, about 96%, about 97%, about 98%, about 99%, 99.5%, or about 100% by weight as determined by HPLC.

In embodiments, the oxalate salt of Compound 1 is a hydrate.

### Methods of Manufacturing

Aspects of the present disclosure relate to scalable methods of making Compound 1.

In embodiments, the methods of making Compound 1 comprise reacting a bis-oxyma linker with psilocin to form Compound 1 according to scheme A shown below:

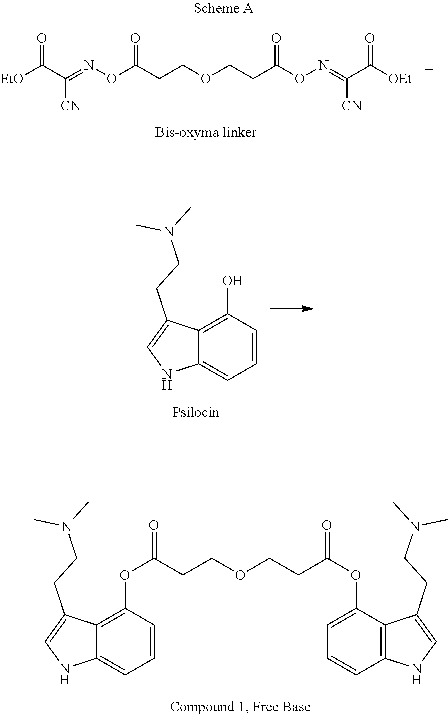

In embodiments, the step of reacting the bis-oxyma linker with psilocin is performed in the presence of an organic solvent. In embodiments, the solvent is ethyl acetate (EtOAc).

In embodiments, the step of reacting the bis-oxyma linker with psilocin is performed in the presence of a base. In embodiments, the base is triethylamine (TEA).

In embodiments, the step of reacting the bis-oxyma linker with psilocin is performed at a temperature of about 22° C.

In embodiments, the methods comprise adding the bis-oxyma linker to a suspension of psilocin.

In embodiments, the step of reacting the bis-oxyma linker with psilocin comprises reacting 2 equivalents of psilocin with 1 equivalent of the bis-oxyma linker.

In embodiments, the psilocin is formed according to the scheme shown below.

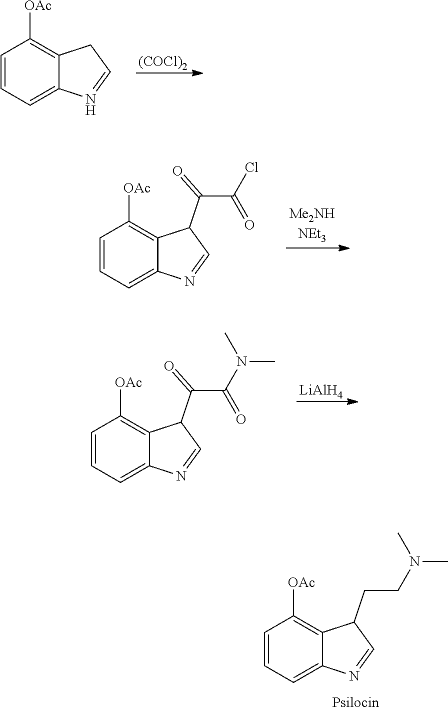

In embodiments, the psilocin is prepared by a method comprising:
i) reacting 4-acetoxyindole with oxalyl chloride to form 3-(2-chloro-2-oxoacetyl)-1H-indol-4-yl acetate;
ii) reacting 3-(2-chloro-2-oxoacetyl)-1H-indol-4-yl acetate with dimethylamine to form 3-([(dimethylcarbamoyl)carbonyl])-1H-indol-4-yl acetate; and
iii) reacting 3-([(dimethylcarbamoyl)carbonyl])-1H-indol-4-yl acetate with lithium aluminum hydride to form the psilocin.

In embodiments, the reacting in step (ii) is performed in the presence of TEA.

In embodiments, the bis-oxyma linker is formed according to the scheme shown below.

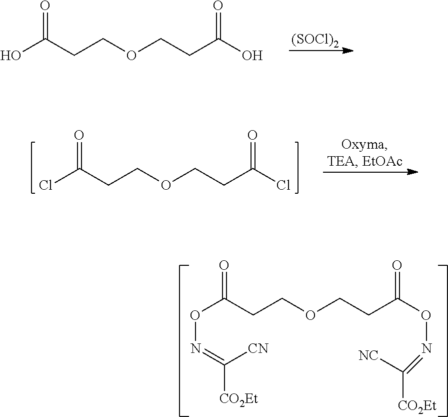

In embodiments, the methods of making bis-oxyma linker comprise:
(i) reacting 3,3′-oxydipropionic acid with thionyl dichloride to form 3,3′-oxydipropionyl dichloride; and
(ii) reacting 3,3′-oxydipropionyl dichloride with ethyl 2-cyano-2-(hydroxyimino)acetate in the presence of a base and a solvent to form the bis-oxyma linker.

In embodiments, the base in step (ii) is triethylamine (TEA). In embodiments, the solvent in step (ii) is ethyl acetate (EtOAc).

In embodiments, the present disclosure provides a method of manufacturing the pharmaceutically acceptable salt of compound 1, comprising:
(i) reacting the bis-oxyma linker with psilocin in the presence of a base and a solvent to form free base Compound 1; and
(ii) reacting the free base Compound 1 with an acid to form the pharmaceutically acceptable salt.

In embodiments, the base in step (i) is triethylamine (TEA). In embodiments, the solvent in step (i) is ethyl acetate (EtOAc). In embodiments, the reacting in (ii) is performed in the presence of a solvent. In embodiments, the solvent is a mixture of isopropyl alcohol and water. In embodiments, the method further comprises (iii) recrystallizing the pharmaceutically acceptable salt. In embodiments, the acid is monofumaric acid, sesquifumaric acid, succinic acid, L-tartric acid, hydrochloric acid, pyruvic acid, oxodipropanoic acid, adipate salt or oxalic acid.

In embodiments, the present disclosure provides a compound having the following structure (A):

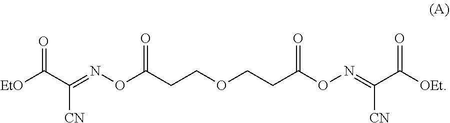 [SMILES: CCOC(=O)/C(C#N)=N/OC(=O)CCOCCC(=O)O/N=C(\C#N)C(=O)OCC]

### Pharmaceutical Compositions

The present disclosure provides pharmaceutical compositions comprising at least one salt form of Compound 1 (e.g., monofumarate salt, a sesquifumarate salt, bis-formate salt, succinate salt, hydrochloride salt of compound 1, pyruvate salt of compound 1, oxodipropanoate salt of compound 1, adipate salt of compound 1, or oxalate salt of compound 1, or L-tartrate salt of Compound 1) disclosed herein and one or more pharmaceutically acceptable excipients.

In embodiments, the pharmaceutical composition comprises a monofumarate salt of Compound 1 and one or more pharmaceutically acceptable excipients.

In embodiments, the pharmaceutical composition comprises a sesquifumarate salt of Compound 1 and one or more pharmaceutically acceptable excipients.

In embodiments, the pharmaceutical composition comprises a bis-formate salt of Compound 1 and one or more pharmaceutically acceptable excipients.

In embodiments, the pharmaceutical composition comprises a succinate salt of Compound 1 and one or more pharmaceutically acceptable excipients.

In embodiments, the pharmaceutical composition comprises a L-tartrate salt of Compound 1 and one or more pharmaceutically acceptable excipients.

In embodiments, the pharmaceutical composition comprises a hydrochloride salt of Compound 1 and one or more pharmaceutically acceptable excipients.

In embodiments, the pharmaceutical composition comprises a pyruvate salt of Compound 1 and one or more pharmaceutically acceptable excipients.

In embodiments, the pharmaceutical composition comprises a 3-oxodipropanoate salt of Compound 1 and one or more pharmaceutically acceptable excipients.

In embodiments, the pharmaceutical composition comprises an adipate salt of Compound 1 and one or more pharmaceutically acceptable excipients.

In embodiments, the pharmaceutical composition comprises an oxalate salt of Compound 1 and one or more pharmaceutically acceptable excipients.

The salt forms provided herein may be administered as salts per se or may be formulated as pharmaceutical compositions. The pharmaceutical compositions may comprise one or more pharmaceutically acceptable excipients, such as carriers, diluents, fillers, disintegrants, lubricating agents, binders, colorants, pigments, stabilizers, preservatives, and/or antioxidants.

The pharmaceutical compositions can be formulated by techniques known to the person skilled in the art, such as the techniques published in “Remington: The Science and Practice of Pharmacy”, Pharmaceutical Press, 22nd edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

### Methods of Treatment

The disclosure further relates to salt forms of Compound 1 disclosed herein, or a pharmaceutical composition comprising at least one salt form of Compound 1 disclosed herein, for use in the treatment of a serotonin 5-HT2A receptor associated disease/disorder. In embodiments, the compounds may be used in the treatment of an anxiety disorder, attention deficit hyperactivity disorder (ADHD), depression (including treatment resistant depression), cluster headache, diminished drive, burn-out, bore-out, migraine, Parkinson's disease, schizophrenia, an eating disorder (including anorexia nervosa), psychotic disorder, schizophrenia, schizophreniform disorder, schizoaffective disorder, bipolar I disorder, bipolar II disorder, major depressive disorder, psychotic depression, delusional disorders, shared psychotic disorder, Shared paranoia disorder, brief psychotic disorder, paranoid personality disorder, schizoid personality disorder, schizotypal personality disorder, social anxiety disorder, substance-induced anxiety disorder, selective mutism, panic disorder, panic attacks, agoraphobia, posttraumatic stress disorder (PTSD), premenstrual dysphoric disorder (PMDD), and premenstrual syndrome (PMS).

In embodiments, the 5-HT2A receptor associated disease or disorder is depression. In embodiments, the depression is treatment resistant depression.

In embodiments, the 5-HT2A receptor associated disease or disorder is an eating disorder. In embodiments, the eating disorder is anorexia nervosa.

In embodiments, the 5-HT2A receptor associated disease or disorder is an anxiety disorder.

In embodiments, the 5-HT2A receptor associated disease or disorder is bipolar I disorder.

In embodiments, the 5-HT2A receptor associated disease or disorder is bipolar II disorder.

In embodiments, the 5-HT2A receptor associated disease or disorder is major depressive disorder.

In embodiments, the 5-HT2A receptor associated disease or disorder is posttraumatic stress disorder (PTSD).

From the foregoing, it will be appreciated that specific embodiments of the invention have been described herein for purposes of illustration, but that various modifications may be made without deviating from the scope of the invention. Accordingly, the invention is not limited except as by the appended claims.

### EXAMPLES

The present invention is further illustrated by reference to the following Examples. However, it is noted that that Examples, like the embodiments described above, are illustrative and are not to be construed as restricting the scope of the invention in any way.

### Example 1: Salt Screen of Compound 1

### A. Synthesis of Compound 1

The free base of Compound 1 was synthesized according to Schemes 1-3.

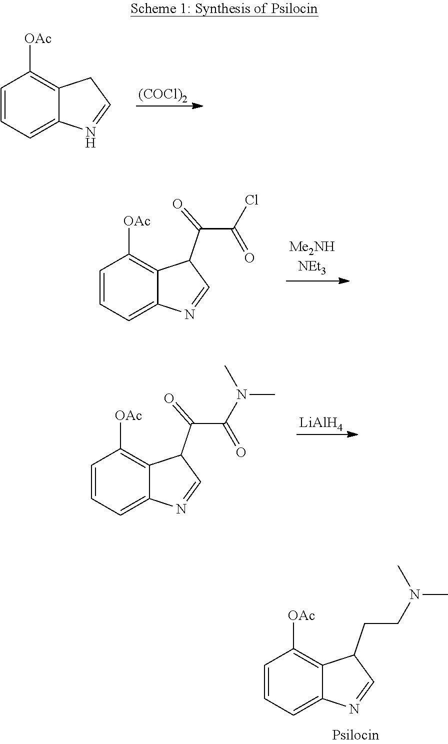

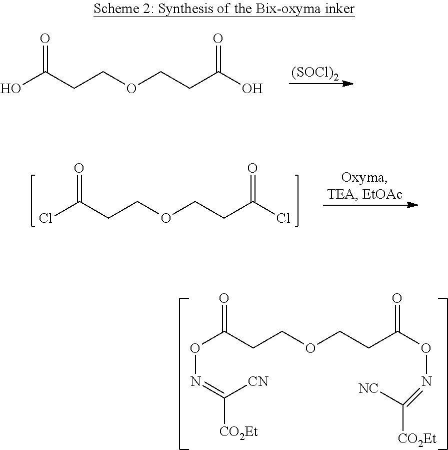

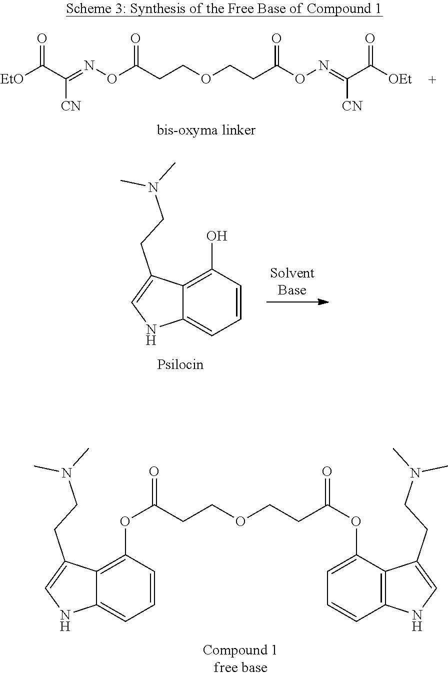

Psilocin was synthesized from 4-acetoxyindole according to Scheme 1. Oxalyl chloride was added slowly to a stirred mixture of tetrahydrofuran (THF) and t-butyl methyl ether (TBME) maintaining the temperature at 15-25° C. The reaction was warmed and a solution of 4-acetoxyindole dissolved in a mixture of THF and TBME was added. The reaction was stirred for at least 1.5 h. The reaction was cooled to −10 to 9° C. and a mixture of dimethylamine and triethylamine was added dropwise while maintaining the temperature between −10 to 9° C. The reaction was then stirred for at least 1.5 h at −10 to 9° C. The product was filtered, washed successively with a mixture of THF/TBME followed by n-heptane and dried. The crude product was further purified by a slurry in water at 15-25° C. and then dried in a vacuum oven at 60° C. The crude product was slurried in THF and cooled to 0-10° C. A THF solution of 2.4 M lithium aluminium hydride (LiAlH₄) was added dropwise while maintaining the temperature between 0-20° C. The reaction was stirred for a further 30-60 minutes at 15-25° C. then warmed to 60-65° C. and stirred for at least 12 hours. The reaction was cooled to 0-10° C. and the excess LiAlH₄ quenched by addition of acetone followed by aqueous citric acid solution while maintaining temperature between 0-30° C. The mixture was stirred for at least 1 h at 15-25° C., then filtered and the filter cake was washed with THE. The filter cake was slurried with a mixture of THF and aqueous citric acid solution for at least 2 hours. The batch was filtered, and the filter cake was washed with THE. The combined filtrates were concentrated at a maximum temperature of 50° C. then filtered through a silica pad. The pad was eluted with THF and the product containing fractions evaporated at a maximum temperature of 50° C. The resulting solid was slurried in isopropyl acetate (iPrOAc):TBME mixture, stirred at 15-25° C. for at least 2 hours, filtered and washed with TBME. The solid was dried under vacuum at 40° C.

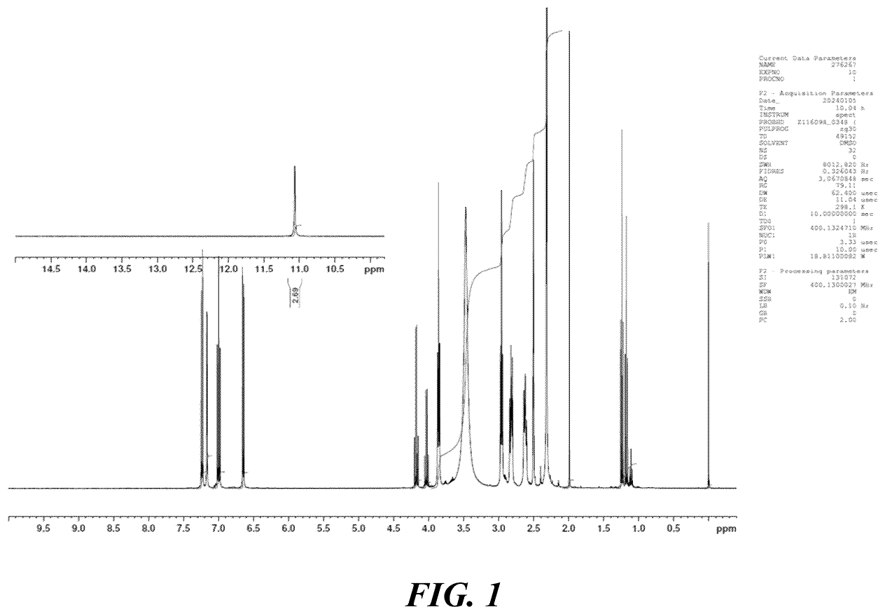

A 400 mL reactor vessel was charged with psilocin (35.51 g, 0.174 mol, circa 1.98 eq) and EtOAc (150 mL) and the resulting suspension stirred at 400 rpm under a nitrogen atmosphere with the reaction mixture held at 22° C. Bis-oxyma linker was synthesized according to Scheme 2. Briefly, 3,3′-oxydipropionic acid with thionyl dichloride to form 3,3′-oxydipropionyl dichloride. 3,3′-oxydipropionyl dichloride was then reacted with ethyl 2-cyano-2-(hydroxyimino)acetate in the presence of triethylamine (TEA) and ethyl acetate (EtOAc) to form the bis-oxyma linker. A solution of bis-oxyma linker (73% pure by qNMR analysis, 49.30 g, 87.7 mmol, 1.00 eq) in EtOAc (200 mL) was added in one portion and the resulting mixture stirred at 22° C. for 15 min, giving a red/brown suspension. The stirrer speed was increased to 600 rpm and stirring continued for a further 75 min. A second portion of psilocin (1.06 g, 5.19 mmol, circa 0.06 eq) was added in one portion and the resulting suspension stirred for 30 min. TEA (50 mL, 0.359 mol, 4.09 eq) was then added all at once and the reaction mixture stirred for 30 min until all solids had dissolved. The resulting orange/red solution was transferred to a separating funnel and the reaction vessel rinsed with EtOAc (175 mL). The resulting solution was washed sequentially with water (175 mL, measured to be at pH=9/10) and saturated brine (175 mL), then dried over magnesium sulfate, and filtered through filter paper with the filter cake being rinsed with EtOAc (50 mL). The resulting red/brown filtrate solution was concentrated in vacuo on a rotary evaporator with the water bath set at 31° C. with a max vacuum of 40 mbar to generate the free base of Compound 1 (40.27 g, 86%). ¹H NMR is shown in FIG. 1. The free base of Compound 1 was a brown solid.

### B. Bis-Formate Salt of Compound 1

### Characterization of Bis-Formate Salt of Compound 1:

A portion of the free base of Compound 1 was purified by chromatography using an Interchim Puriflash C18 column with a 5-95% acetonitrile in water linear gradient with 0.1 v/v % formic acid as a pH modifier. The relevant fractions were freeze-dried to give an amorphous bis-formate salt of Compound 1.

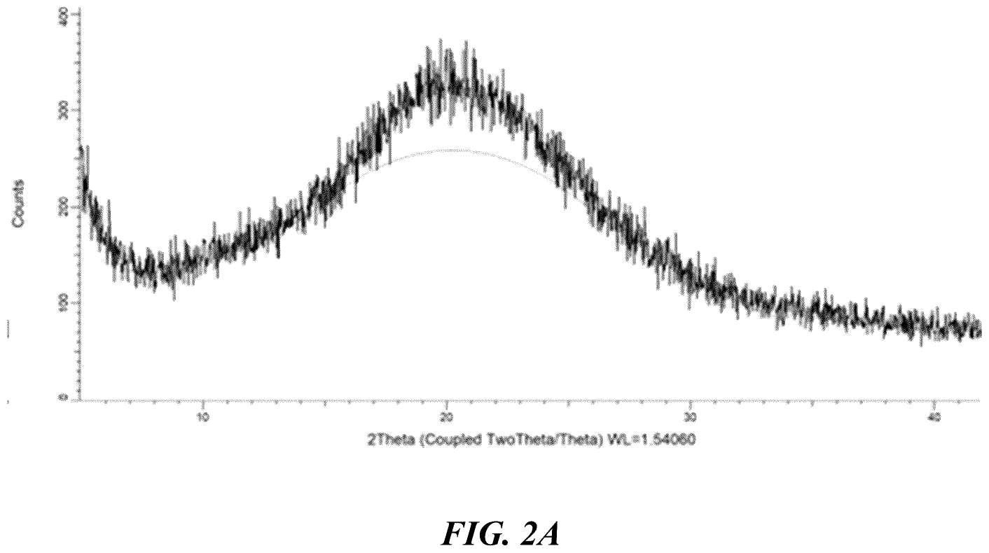

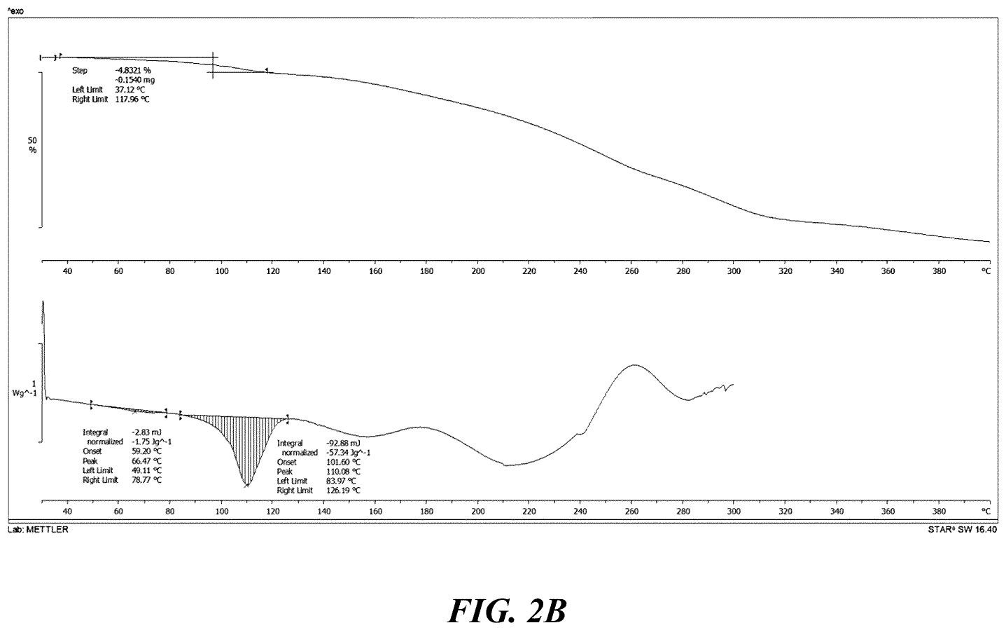

The bis-formate Salt of Compound 1 was obtained as an off white solid. It was characterized by XRPD and found to be amorphous (FIG. 2A). Thermal analysis by differential scanning calorimetry (DSC) showed endothermic events between 49-79° C., 84-126° C. and no evidence of recrystallisation, thermogravimetric Analysis (TGA) showed 4.8% mass loss between 30-118° C., followed by further mass loss (FIG. 2B).

### Solid State Stability of Bis-Formate Salt:

Portions of the bis-formate salt of Compound 1, with an initial UPLC purity of 99.5%, were placed in storage under the following conditions;
Storage at 40° C./75% humidity for 7 days
Storage at 25° C./96% humidity for 7 days

After 7 days both samples were analyzed by UPLC, and showed degradation.
After storage at 40° C./75% relative humidity for 7 days—UPLC purity 39%;
After storage at 25° C./96% relative humidity for 7 days—UPLC purity 80%

### Solution Stability of Bis-Formate Salt:

An initial investigation into the solution stability of bis-formate salt in PBS at pH 7.4 and 5.8 and in acetonitrile:water (1:1) at 2, 4, and 44 hours was carried out at room temperature. The results are summarized in Table 1.

TABLE 1
Solution Stability of Bis-Formate Salt of Compound 1

Purity, area %

| Media | T = O | T = 2 hr | T = 4 hr | T = 44 hr |
|---|---|---|---|---|
| Acetonitrile:water | 97.6% | 97.0% | 96.9% | 92.8% |
| PBS at pH 7.4 | 98.2% | 98.2% | 97.9% | 91.9% |
| PBS at pH 5.8 | 98.1% | 98.2% | 98.3% | 98.1% |

### C. Fumarate (1.0 Equivalents) Salt of Compound 1

### Initial Preparation of the Fumarate (1.0 Equivalents) of Compound 1:

The bis-formate salt of Compound 1 (490 mg) was dissolved in a mixture of water and acetonitrile (1:1, 30 mL). A solution of fumaric acid (90.8 mg, 1 equivalent) in a mixture of water and acetonitrile (1:1, 25 mL) was added, followed by additional water (10 mL). The resultant clear solution was flash frozen in a dry ice/acetone bath and then lyophilized to give the monofumarate salt of Compound 1 (534.5 mg). To remove residual formic acid, the salt was redissolved in a mixture of water and acetonitrile (1:1, 20 mL) and further water (5 mL) was added. The solution was flash frozen in a dry ice/acetone bath and then lyophilized to give the monofumarate salt of Compound 1 (524.8 mg) as an amorphous beige solid. The ¹H NMR was consistent with 1 equivalent of fumaric acid (FIG. 3A). UPLC purity 98.7%.

### Alternative Preparation of Fumarate (1.0 Equivalents) Salt of Compound 1:

200.49 mg fumarate (1 equivalent) amorphous salt of Compound 1 was weighed into a 7 mL vial and treated with acetonitrile (4 mL). The vial was capped, and the lid sealed with Parafilm®. The vial was wrapped in foil. The mixture (solvent+gum) was shaken at room temperature overnight. After shaking at room temperature overnight microscopy showed crystalline material.

A small portion was removed and analyzed by XRPD. The material was broken up slightly to loosen the layer on the bottom of the vial and shaking at room temperature was continued for a further 2 hours. The solid was isolated by filtration and dried briefly by suction and in vacuo (30 minutes) to give an off-white solid (173.77 mg).

### Characterization of the Fumarate (1.0 Equivalents) Salt of Compound 1:

The fumarate (1.0 equivalents) salt of Compound 1 was characterized by ¹H NMR, UPLC, XRPD, DSC, TGA and GVS.

Analysis by XRPD shows the material to be crystalline (FIG. 3B, Table 2A).

TABLE 2A
Diffraction Peaks for the Fumarate (1.0 equivalents)
Salt of Compound 1 (“Form 1”)

| Peak number | Peak Position °2θ | Relative Intensity |
|---|---|---|
| Peak 1 | 5.266 | 11.34% |
| Peak 2 | 7.225 | 24.13% |
| Peak 3 | 7.663 | 4.88% |
| Peak 4 | 8.921 | 15.20% |
| Peak 5 | 10.441 | 33.56% |
| Peak 6 | 11.328 | 3.44% |
| Peak 7 | 12.777 | 45.24% |
| Peak 8 | 13.718 | 2.41% |
| Peak 9 | 14.424 | 7.27% |
| Peak 10 | 14.674 | 10.87% |
| Peak 11 | 15.364 | 15.15% |
| Peak 12 | 16.468 | 8.66% |
| Peak 13 | 16.865 | 19.21% |
| Peak 14 | 17.345 | 8.17% |
| Peak 15 | 17.853 | 40.66% |
| Peak 16 | 18.722 | 100.00% |
| Peak 17 | 19.343 | 24.48% |
| Peak 18 | 19.768 | 9.10% |
| Peak 19 | 20.756 | 9.06% |
| Peak 20 | 21.024 | 6.36% |
| Peak 21 | 21.417 | 35.02% |
| Peak 22 | 21.825 | 15.97% |
| Peak 23 | 22.791 | 8.61% |
| Peak 24 | 23.213 | 84.94% |
| Peak 25 | 24.474 | 6.25% |
| Peak 26 | 24.832 | 13.77% |
| Peak 27 | 25.719 | 2.20% |
| Peak 28 | 26.383 | 12.23% |
| Peak 29 | 27.434 | 3.71% |
| Peak 30 | 28.59 | 1.57% |
| Peak 31 | 29.034 | 3.41% |
| Peak 32 | 29.478 | 2.22% |
| Peak 33 | 30.154 | 1.93% |
| Peak 34 | 30.618 | 2.15% |
| Peak 35 | 31.336 | 3.14% |
| Peak 36 | 32.314 | 1.15% |
| Peak 37 | 32.688 | 4.60% |

¹H NMR of the fumarate (1.0 equivalents) salt of Compound 1 is consistent with structure and shows the presence of ca. 1.0 equivalent of fumarate, 0.04 equivalents of formate, and acetonitrile (0.34 equivalents). UPLC purity 99.5%. Thermal analysis by DSC shows a complex thermal profile with unresolved endothermic events between 88-142° C., endothermic events between 144-155° C. and 156-174° C. and an exothermic event between 175-212° C. (likely decomposition) (FIG. 3C). TGA analysis shows a 4.8% mass loss between 36-180° C., followed by decomposition above ca. 18000 (FIG. 3D).

A GVS experiment to study the behavior of the fumarate (1.0 equivalents) salt of Compound 1 under conditions of varying relative humidity was carried out and shows the sample to be hygroscopic with a 22% increase in mass over 0-90% relative humidity range over the second sorption cycle. A portion was removed and XRPD analysis of the material post GVS shows poorly crystalline material with a change in XRPD pattern (FIGS. 3E-3F). UPLC analysis shows a reduction in purity (to 81.7%) post GVS.

### Solid State Stability of the Fumarate (1.0 Equivalents) Salt of Compound 1:

Portions of the fumarate (1.0 equivalents) salt of Compound 1, with an initial UPLC purity of 99.3%, were placed on storage under the following conditions:
Storage at 40° C./75% relative humidity for 7 days
Storage at 25° C./96% relative humidity for 7 days

After 7 days both samples were analyzed by UPLC and both showed degradation, especially at elevated temperature.
After storage at 40° C./75% relative humidity for 7 days—UPLC purity 57%. Shows a change in XRPD pattern.
After storage at 25° C./96% relative humidity for 7 days—UPLC purity 86.1%

### Polymorphism Assessment of Fumarate (1.0 Equivalents) Salt of Compound 1 in 12 Solvents:

An initial polymorphism assessment of the fumarate (1.0 equivalents) salt of Compound 1 was carried out by slurrying in 12 diverse solvents. Portions of the fumarate (1.0 equivalents) of Compound 1 (each ca. 5 mg) were weighed into vials and treated with solvents to give slurries as described in Table 3. The vials were capped, sealed with Parafilm®, and then covered with foil. The experiments were shaken at room temperature for 4 days. After 4 days all the experiments had remained as slurries apart from the experiment in water. Portions of solid were removed from each one and analyzed by XRPD.

TABLE 3
Initial Polymorphism Assessment of the Fumarate (1.0
equivalents) salt of Compound 1 in 12 solvents

| | | Volume |
|---|---|---|
| Input weight/mg | Solvent | μL |
| 4.78 | TBME | 60 |
| 4.97 | Acetone | 40 |
| 5.03 | Chloroform | 40 |
| 5.09 | THE | 40 |
| 4.99 | Ethyl acetate | 40 |
| 4.93 | Ethanol | 40 |
| 5.45 | Acetonitrile | 40 |
| 5.4 | Heptane | 60 |
| 4.76 | water | 20 |
| 4.82 | toluene | 40 |
| 5.16 | IPA | 40 |
| 5.2 | Benzyl alcohol | 20 |

Several different new XRPD patterns were observed when compared with the XRPD pattern of the input material (FIG. 3G). The polymorphism assessment identified 8 additional polymorphic forms designated as Forms 2-9 (FIGS. 3H-3O, Tables 2B3-2H).

TABLE 2B
Diffraction Peaks for the Fumarate (1.0 equivalents)
Salt of Compound 1 (“Form 2”)

| Peak number | Peak Position °2θ | Relative Intensity |
|---|---|---|
| Peak 1 | 8.694 | 12.85% |
| Peak 2 | 9.611 | 4.13% |
| Peak 3 | 10.402 | 13.58% |
| Peak 4 | 10.943 | 9.76% |
| Peak 5 | 12.658 | 20.97% |
| Peak 6 | 14.627 | 11.27% |
| Peak 7 | 15.825 | 8.87% |
| Peak 8 | 16.699 | 19.62% |
| Peak 9 | 17.992 | 21.19% |
| Peak 10 | 18.64 | 11.97% |
| Peak 11 | 19.051 | 4.43% |
| Peak 12 | 19.273 | 7.50% |
| Peak 13 | 19.7 | 3.27% |
| Peak 14 | 20.061 | 6.86% |
| Peak 15 | 20.749 | 100.00% |
| Peak 16 | 21.435 | 4.40% |
| Peak 17 | 21.983 | 20.49% |
| Peak 18 | 22.921 | 9.62% |
| Peak 19 | 23.44 | 3.73% |
| Peak 20 | 24.04 | 20.02% |
| Peak 21 | 25.507 | 15.93% |
| Peak 22 | 26.252 | 8.18% |
| Peak 23 | 26.611 | 5.83% |
| Peak 24 | 27.418 | 6.40% |
| Peak 25 | 29.272 | 2.53% |
| Peak 26 | 30.346 | 3.87% |
| Peak 27 | 33.785 | 2.28% |
| Peak 28 | 34.867 | 3.20% |
| Peak 29 | 37.726 | 2.30% |

TABLE 2C
Diffraction Peaks for the Fumarate (1.0 equivalents)
Salt of Compound 1 (“Form 3”)

| Peak Number | Peak Position °2θ | Relative Intensity |
|---|---|---|
| Peak 1 | 7.816 | 12.93% |
| Peak 2 | 8.679 | 20.99% |
| Peak 3 | 9.873 | 8.79% |
| Peak 4 | 10.157 | 11.32% |
| Peak 5 | 10.685 | 7.11% |
| Peak 6 | 11.261 | 14.18% |
| Peak 7 | 11.935 | 7.98% |
| Peak 8 | 12.138 | 7.99% |
| Peak 9 | 12.512 | 12.44% |
| Peak 10 | 13.145 | 5.09% |
| Peak 11 | 14.361 | 5.07% |
| Peak 12 | 15.165 | 11.39% |
| Peak 13 | 16.211 | 5.61% |
| Peak 14 | 16.802 | 71.96% |
| Peak 15 | 17.688 | 100.00% |
| Peak 16 | 18.274 | 9.28% |
| Peak 17 | 19.184 | 88.78% |
| Peak 18 | 19.826 | 51.48% |
| Peak 19 | 21.011 | 20.80% |
| Peak 20 | 21.633 | 33.44% |
| Peak 21 | 22.961 | 18.27% |
| Peak 22 | 23.718 | 58.01% |
| Peak 23 | 24.019 | 8.01% |
| Peak 24 | 24.453 | 53.85% |
| Peak 25 | 24.865 | 38.47% |
| Peak 26 | 25.172 | 10.61% |
| Peak 27 | 26.497 | 2.46% |
| Peak 28 | 27.158 | 5.41% |
| Peak 29 | 27.864 | 4.70% |
| Peak 30 | 29.071 | 4.37% |
| Peak 31 | 29.543 | 4.63% |
| Peak 32 | 29.926 | 7.91% |
| Peak 33 | 31.877 | 6.67% |
| Peak 34 | 32.335 | 3.38% |
| Peak 35 | 33.967 | 3.86% |
| Peak 36 | 35.073 | 5.43% |

TABLE 2D
Diffraction Peaks for the Fumarate (1.0 equivalents)
Salt of Compound 1 (“Form 4”)

| Peak number | Peak Position °2θ | Relative Intensity |
|---|---|---|
| Peak 1 | 5.731 | 37.45% |
| Peak 2 | 7.217 | 2.05% |
| Peak 3 | 7.648 | 1.83% |
| Peak 4 | 8.183 | 53.95% |
| Peak 5 | 8.836 | 3.01% |
| Peak 6 | 9.333 | 40.38% |
| Peak 7 | 10.219 | 11.58% |
| Peak 8 | 10.498 | 12.79% |
| Peak 9 | 11.286 | 79.17% |
| Peak 10 | 12.344 | 1.75% |
| Peak 11 | 12.867 | 10.64% |
| Peak 12 | 13.161 | 7.05% |
| Peak 13 | 13.639 | 2.79% |
| Peak 14 | 14.499 | 3.41% |
| Peak 15 | 15.22 | 3.97% |
| Peak 16 | 15.836 | 16.37% |
| Peak 17 | 16.202 | 12.43% |
| Peak 18 | 16.447 | 12.71% |
| Peak 19 | 16.876 | 35.13% |
| Peak 20 | 17.335 | 18.45% |
| Peak 21 | 17.558 | 17.29% |
| Peak 22 | 18.479 | 32.84% |
| Peak 23 | 18.88 | 20.66% |
| Peak 24 | 19.446 | 16.30% |
| Peak 25 | 19.654 | 30.11% |
| Peak 26 | 20.381 | 5.76% |
| Peak 27 | 21.049 | 100.00% |
| Peak 28 | 21.387 | 9.44% |
| Peak 29 | 21.948 | 5.79% |
| Peak 30 | 22.583 | 2.79% |
| Peak 31 | 23.107 | 26.82% |
| Peak 32 | 23.682 | 9.43% |
| Peak 33 | 24.786 | 4.19% |
| Peak 34 | 25.318 | 5.42% |
| Peak 35 | 25.418 | 5.79% |
| Peak 36 | 26.282 | 4.18% |
| Peak 37 | 26.994 | 2.23% |
| Peak 38 | 28.17 | 1.76% |
| Peak 39 | 29.116 | 1.74% |
| Peak 40 | 30.362 | 4.86% |
| Peak 41 | 31.977 | 3.82% |
| Peak 42 | 33.905 | 2.36% |

TABLE 2E
Diffraction Peaks for the Fumarate (1.0 equivalents)
Salt of Compound 1 (“Form 5”)

| Peak Number | Peak Position °2θ | Relative Intensity |
|---|---|---|
| Peak 1 | 7.147 | 12.12% |
| Peak 2 | 7.821 | 14.56% |
| Peak 3 | 8.694 | 100.00% |
| Peak 4 | 9.539 | 2.42% |
| Peak 5 | 10.462 | 21.83% |
| Peak 6 | 11.116 | 7.58% |
| Peak 7 | 11.596 | 6.80% |
| Peak 8 | 12.243 | 19.22% |
| Peak 9 | 12.795 | 17.71% |
| Peak 10 | 14.388 | 38.88% |
| Peak 11 | 15.563 | 10.92% |
| Peak 12 | 16.43 | 4.91% |
| Peak 13 | 16.817 | 14.28% |
| Peak 14 | 17.356 | 18.84% |
| Peak 15 | 17.745 | 8.77% |
| Peak 16 | 18.733 | 45.47% |
| Peak 17 | 19.241 | 48.87% |
| Peak 18 | 19.694 | 56.75% |
| Peak 19 | 20.015 | 18.65% |
| Peak 20 | 20.794 | 11.74% |
| Peak 21 | 21.073 | 13.79% |
| Peak 22 | 21.4 | 18.35% |
| Peak 23 | 21.793 | 26.15% |
| Peak 24 | 22.155 | 21.53% |
| Peak 25 | 22.841 | 16.61% |
| Peak 26 | 23.158 | 33.61% |
| Peak 27 | 23.524 | 54.79% |
| Peak 28 | 24.61 | 8.57% |
| Peak 29 | 25.222 | 16.09% |
| Peak 30 | 26.424 | 11.70% |
| Peak 31 | 26.719 | 4.25% |
| Peak 32 | 28.191 | 3.69% |
| Peak 33 | 29.027 | 20.56% |
| Peak 34 | 33.284 | 3.27% |
| Peak 35 | 34.781 | 4.14% |

TABLE 2F
Diffraction Peaks for the Fumarate (1.0 equivalents)
Salt of Compound 1 (“Form 6”)

| Peak Number | Peak Position °2θ | Relative Intensity |
|---|---|---|
| Peak 1 | 7.291 | 100.00% |
| Peak 2 | 7.995 | 6.05% |
| Peak 3 | 8.35 | 8.35% |
| Peak 4 | 8.862 | 5.97% |
| Peak 5 | 10.092 | 2.72% |
| Peak 6 | 10.558 | 66.01% |
| Peak 7 | 11.598 | 32.67% |
| Peak 8 | 12.093 | 11.78% |
| Peak 9 | 12.425 | 11.73% |
| Peak 10 | 13.703 | 7.61% |
| Peak 11 | 13.92 | 28.93% |
| Peak 12 | 14.553 | 5.58% |
| Peak 13 | 15.094 | 5.35% |
| Peak 14 | 15.456 | 11.30% |
| Peak 15 | 15.814 | 18.41% |
| Peak 16 | 16.053 | 16.74% |
| Peak 17 | 16.292 | 5.10% |
| Peak 18 | 17.997 | 30.94% |
| Peak 19 | 18.258 | 18.86% |
| Peak 20 | 18.819 | 20.43% |
| Peak 21 | 19.198 | 22.58% |
| Peak 22 | 19.839 | 52.24% |
| Peak 23 | 20.414 | 43.15% |
| Peak 24 | 20.896 | 25.38% |
| Peak 25 | 21.849 | 9.21% |
| Peak 26 | 22.177 | 20.58% |
| Peak 27 | 22.45 | 27.93% |
| Peak 28 | 23.094 | 44.36% |
| Peak 29 | 23.577 | 42.49% |
| Peak 30 | 24.029 | 33.74% |
| Peak 31 | 24.56 | 8.01% |
| Peak 32 | 25.126 | 11.15% |
| Peak 33 | 25.651 | 19.37% |
| Peak 34 | 26.071 | 6.26% |
| Peak 35 | 26.944 | 5.22% |
| Peak 36 | 28.303 | 4.93% |
| Peak 37 | 29.235 | 10.71% |
| Peak 38 | 31.02 | 5.21% |
| Peak 39 | 31.622 | 4.13% |
| Peak 40 | 33.215 | 2.32% |
| Peak 41 | 34.922 | 3.02% |
| Peak 42 | 36.768 | 2.57% |

TABLE 2G
Diffraction Peaks for the Fumarate (1.0 equivalents)
Salt of Compound 1 (“Form 7”)

| Peak Number | Peak Position °2θ | Relative Intensity |
|---|---|---|
| Peak 1 | 5.253 | 22.31% |
| Peak 2 | 7.183 | 31.71% |
| Peak 3 | 8.724 | 59.38% |
| Peak 4 | 8.928 | 24.91% |
| Peak 5 | 10.199 | 34.92% |
| Peak 6 | 10.518 | 66.98% |
| Peak 7 | 11.475 | 5.99% |
| Peak 8 | 11.758 | 11.81% |
| Peak 9 | 12.226 | 16.23% |
| Peak 10 | 12.806 | 22.05% |
| Peak 11 | 14.131 | 21.01% |
| Peak 12 | 15.236 | 26.42% |
| Peak 13 | 16.473 | 15.17% |
| Peak 14 | 17.19 | 80.05% |
| Peak 15 | 17.862 | 51.20% |
| Peak 16 | 18.192 | 54.23% |
| Peak 17 | 18.748 | 90.34% |
| Peak 18 | 19.416 | 54.33% |
| Peak 19 | 19.747 | 76.74% |
| Peak 20 | 20.761 | 12.45% |
| Peak 21 | 21.123 | 32.13% |
| Peak 22 | 21.447 | 75.17% |
| Peak 23 | 21.933 | 48.60% |
| Peak 24 | 23.242 | 67.15% |
| Peak 25 | 23.588 | 100.00% |
| Peak 26 | 23.978 | 27.37% |
| Peak 27 | 24.871 | 25.35% |
| Peak 28 | 25.18 | 86.24% |
| Peak 29 | 26.449 | 25.54% |
| Peak 30 | 27.486 | 6.78% |
| Peak 31 | 29.154 | 15.66% |
| Peak 32 | 30.269 | 8.66% |
| Peak 33 | 38.051 | 6.21% |

TABLE 2H
Diffraction Peaks for the Fumarate (1.0 equivalents)
Salt of Compound 1 (“Form 8”)

| Peak Number | Peak Position °2θ | Relative Intensity |
|---|---|---|
| Peak 1 | 5.602 | 88.19% |
| Peak 2 | 7.133 | 3.37% |
| Peak 3 | 8.084 | 14.51% |
| Peak 4 | 8.692 | 12.42% |
| Peak 5 | 9.267 | 19.45% |
| Peak 6 | 10.371 | 4.86% |
| Peak 7 | 11.206 | 100.00% |
| Peak 8 | 12.737 | 7.25% |
| Peak 9 | 13.18 | 6.64% |
| Peak 10 | 14.449 | 3.39% |
| Peak 11 | 14.955 | 3.65% |
| Peak 12 | 15.769 | 9.17% |
| Peak 13 | 16.835 | 24.21% |
| Peak 14 | 18.621 | 19.48% |
| Peak 15 | 19.329 | 18.39% |
| Peak 16 | 19.677 | 30.58% |
| Peak 17 | 20.808 | 5.96% |
| Peak 18 | 21.141 | 20.26% |
| Peak 19 | 21.837 | 5.52% |
| Peak 20 | 22.48 | 3.58% |
| Peak 21 | 23.229 | 8.38% |
| Peak 22 | 23.532 | 21.51% |
| Peak 23 | 25.145 | 10.17% |
| Peak 24 | 29.124 | 2.58% |
| Peak 25 | 29.467 | 3.34% |

TABLE 21
Diffraction Peaks for the Fumarate (1.0 equivalents)
Salt of Compound 1 (“Form 9”)

| Peak Number | Peak Position °2θ | Relative Intensity |
|---|---|---|
| Peak 1 | 5.508 | 16.11% |
| Peak 2 | 6.104 | 1.13% |
| Peak 3 | 8.183 | 3.71% |
| Peak 4 | 8.841 | 1.57% |
| Peak 5 | 9.647 | 1.45% |
| Peak 6 | 10.024 | 3.67% |
| Peak 7 | 10.844 | 48.16% |
| Peak 8 | 11.39 | 6.55% |
| Peak 9 | 12.116 | 10.24% |
| Peak 10 | 12.904 | 2.55% |
| Peak 11 | 13.205 | 1.42% |
| Peak 12 | 14.631 | 1.34% |
| Peak 13 | 16.218 | 100.00% |
| Peak 14 | 17.04 | 7.88% |
| Peak 15 | 17.742 | 11.69% |
| Peak 16 | 18.166 | 14.50% |
| Peak 17 | 19.092 | 1.64% |
| Peak 18 | 19.903 | 8.68% |
| Peak 19 | 20.393 | 5.27% |
| Peak 20 | 20.691 | 3.44% |
| Peak 21 | 21.605 | 36.48% |
| Peak 22 | 22.713 | 17.27% |
| Peak 23 | 23.467 | 4.20% |
| Peak 24 | 24.237 | 4.25% |
| Peak 25 | 25.321 | 2.97% |
| Peak 26 | 27.896 | 3.10% |
| Peak 27 | 28.609 | 1.31% |
| Peak 28 | 29.473 | 0.61% |
| Peak 29 | 32.527 | 2.75% |
| Peak 30 | 33.294 | 1.28% |

### D. Sesquifumarate (1.5 Equivalents of Fumarate) of Compound 1

The free base of Compound 1 (11.43 g, 21.4 mmol, 1.00 eq) was dissolved in a mixture of 2-propanol (60 mL) and water (15 mL) to give a dark orange solution. Fumaric acid (4.97 g, 42.8 mmol, 2.00 eq) was added and the resulting suspension stirred at room temperature under nitrogen for 18 h to give a red/brown suspension. The suspended solid was collected by filtration on a P3 sintered-glass funnel, and the filter cake rinsed with 2:1 2-propanol/water (20 mL) then suction dried under a stream of nitrogen for 1 h to give an off-white solid that was further dried in a vacuum oven at 35° C./6 mbar for 20 h then at 50° C./6 mbar for 3 days to give sesquifumarate salt of Compound 1 (11.33 g, 75%) as a crystalline off-white solid.

The sesquifumarate (1.5 equivalents) salt of Compound 1 was characterized by ¹H NMR, UPLC, XRPD, DSC, TGA and GVS.

Analysis by XRPD shows the material to be crystalline (FIG. 4A, Table 4).

TABLE 4
Diffraction Peaks for the Fumarate (1.5
equivalents) Salt of Compound 1

| Peak | Peak | Relative |
|---|---|---|
| number | Position °2θ | Intensity |
| Peak 1 | 7.854 | 18.1% |
| Peak 2 | 8.725 | 76.9% |
| Peak 3 | 9.559 | 3.5% |
| Peak 4 | 11.117 | 18.4% |
| Peak 5 | 11.623 | 7.2% |
| Peak 6 | 12.294 | 26.4% |
| Peak 7 | 13.975 | 4.8% |
| Peak 8 | 14.401 | 66.2% |
| Peak 9 | 15.612 | 24.7% |
| Peak 10 | 16.33 | 4.6% |
| Peak 11 | 16.828 | 24.5% |
| Peak 12 | 17.16 | 7.3% |
| Peak 13 | 17.399 | 18.1% |
| Peak 14 | 17.784 | 6.8% |
| Peak 15 | 18.024 | 6.1% |
| Peak 16 | 18.388 | 5.4% |
| Peak 17 | 18.711 | 24.5% |
| Peak 18 | 19.237 | 74.0% |
| Peak 19 | 19.728 | 100.0% |
| Peak 20 | 20.052 | 38.7% |
| Peak 21 | 20.84 | 18.2% |
| Peak 22 | 21.067 | 21.9% |
| Peak 23 | 21.366 | 10.7% |
| Peak 24 | 21.799 | 55.6% |
| Peak 25 | 22.188 | 60.2% |
| Peak 26 | 22.499 | 13.4% |
| Peak 27 | 22.852 | 12.6% |
| Peak 28 | 23.192 | 30.5% |
| Peak 29 | 23.558 | 85.6% |
| Peak 30 | 24.332 | 12.3% |
| Peak 31 | 24.603 | 20.3% |
| Peak 32 | 25.292 | 28.1% |
| Peak 33 | 26.387 | 5.9% |
| Peak 34 | 26.81 | 7.6% |
| Peak 35 | 27.374 | 3.0% |
| Peak 36 | 28.188 | 8.2% |
| Peak 37 | 29.002 | 14.2% |
| Peak 38 | 29.485 | 4.3% |
| Peak 39 | 29.718 | 6.8% |
| Peak 40 | 30.051 | 3.4% |
| Peak 41 | 30.361 | 5.7% |
| Peak 42 | 30.89 | 2.1% |
| Peak 43 | 31.63 | 7.0% |
| Peak 44 | 33.212 | 5.9% |
| Peak 45 | 34.035 | 2.5% |
| Peak 46 | 34.705 | 2.9% |
| Peak 47 | 34.872 | 3.0% |
| Peak 48 | 35.38 | 2.3% |
| Peak 49 | 38.446 | 3.9% |
| Peak 50 | 39.004 | 2.7% |
| Peak 51 | 39.891 | 1.5% |

¹H NMR of the fumarate (1.5 equivalents) salt of Compound 1 is consistent with structure and shows the presence of ca. 1.5 equivalents of fumarate (FIG. 4B). UPLC purity 99.5%. Thermal analysis by DSC shows an endothermic event of onset 168° C. (likely melt), and a broad exothermic event between 176-221° C. (likely decomposition) (FIG. 4C). TGA analysis shows a 2% mass loss between 37-150° C., followed by decomposition above ca. 180° C. (FIG. 4D).

A GVS experiment to study the behavior of the fumarate (1.5 equivalents) salt of Compound 1 under conditions of varying relative humidity was carried out and shows a reversible profile with a 1.8% increase in mass over 0-90% relative humidity range over the second sorption cycle (FIG. 4E). XRPD analysis of the material post GVS shows material with no change in XRPD pattern. UPLC analysis shows no loss in purity post GVS (FIG. 4F).

### Stability Studies of the Fumarate (1.5 Equivalents) Salt of Compound 1

Storage at −20° C.: The batch of the fumarate (1.5 equivalents) salt of Compound 1 was stored in the freezer at −20° C. Samples were removed and analyzed by XRPD and UPLC after 3, 13 and 17 weeks. No change was observed by XRPD after 17 weeks (FIG. 4G). UPLC results are shown in Table 5.

TABLE 5
UPLC Purity After Storage in Freezer (−20° C.)

| Time point | UPLC Purity Storage in freezer (−20° C.) |
|---|---|
| Input | 99.5% |
| 3 weeks | 99.5% |
| 13 weeks | 99.5% |
| 17 weeks | 99.3% |

Storage at 40° C./75% relative humidity and 25° C./96% relative humidity: Portions of the fumarate (1.5 equivalents) salt of Compound 1 were stored under stress conditions of 40° C./75% relative humidity and 25° C./96% relative humidity. Samples were removed after 1 week, 4 weeks, 8 weeks and 12 weeks and analyzed by XRPD and UPLC. No change was observed by XRPD after 12 weeks (FIG. 4H). UPLC results are shown in Table 6.

TABLE 6
UPLC Purity After Storage Under Stress Conditions

| | UPLC Purity | UPLC Purity |
|---|---|---|
| Time point | Storage at 40° C./ 75% RH | Storage at 25° C./ 96% RH |
| Input | 99.5% | 99.5% |
| 7 days | 99.4% | 99.4% |
| 4 weeks | 99.2% | 99.2% |
| 8 weeks | 99.2% | 99.4% |
| 12 weeks | 99.3% | 99.3% |

### Polymorphism Assessment of the Fumarate (1.5 Equivalents) Salt of Compound 1 in 12 Solvents:

An initial polymorphism assessment of the fumarate (1.5 equivalents) salt of Compound 1 was carried out by slurrying in 12 diverse solvents. Portions of the fumarate (1.5 equivalents) salt of Compound 1 (each ca. 10 mg) were weighed into vials and treated with solvents to give slurries. The vials were capped, sealed with Parafilm® and then covered with foil. The experiments were shaken at room temperature for 3 days. After 3 days all the experiments had remained as slurries. Portions were removed from each one and analyzed by XRPD. All gave XRPD patterns consistent with the XRPD pattern of the input material as shown in the overlay in FIG. 4I.

Single crystals of the fumarate (1.5 equivalents) salt of Compound 1 were prepared (FIG. 4J-4L). The structure shows that water is incorporated into the crystal lattice with one molecule of water per molecule of the fumarate (1.5 equivalents) salt of Compound 1. It is evident that the asymmetric unit contains a doubly protonated Compound 1 base molecule and two associated acid moieties originating from different fumaric acid molecules. In one case, the fumaric acid is doubly ionized and in the other fumaric molecule the opposing acid moiety is associated with a third fumaric acid molecule. By implication, for every two molecules of Compound 1 base exists three molecules of fumaric acid, giving a stoichiometry of 1.5 molecules of fumaric acid per unit Compound 1.

### Example 2: Salt Screen

A range of different pharmaceutically acceptable acids were chosen for the salt screening studies (Table 7).

TABLE 7
Acid Selected for Salt Screening Studies

| | | Number of |
|---|---|---|
| Acid | pKₐs | equivalents |
| Hydrobromic acid | −9 | 2 |
| Sulfuric acid | −3, 1.92 | 1, 2 |
| P-Toluenesulfonic acid | −1.34 | 2 |
| Methanesulfonic acid * | −1.2 | 2 |
| Aspartic acid | 1.88, 3.9, 9.9 | 2 |
| Maleic acid | 1.92, 6.23 | 1, 2 |
| L-tartaric acid | 3.02, 4.36 | 1, 2 |
| Succinic acid | 4.2 | 1, 2 |
| Benzoic acid | 4.2 | 2 |
| Acetic acid | 4.76 | 2 |
| Undecanoic acid | 4.95 | 2 |

Experiments were carried out in the following solvents: acetone, tetrahydrofuran (THF), acetonitrile (MeCN), isopropyl alcohol (IPA), and IPA/water mixture.

From these experiments, crystalline succinate salt of Compound 1 was obtained in acetone, THF, IPA/water mixture, and IPA and crystalline L-tartrate salt was obtained in the IPA/water the details of which are provided below. There was no evidence for the formation of crystalline material in any of the other experiments.

### A. Succinate Salt of Compound 1

The free base of Compound 1 was dissolved in acetone (3 mL) and treated with succinic acid in acetone. The reaction was shaken at room temperature overnight. For the experiment with 2 equivalents of succinic acid, the supernatant was decanted, and the residual material was divided into two portions. One portion was titrated with TBME. The second portion was dissolved in water (25 μL) and then IPA (100 μL) was added, and an oil formed. This was treated with a small quantity of the sesquifumarate salt of Compound 1 with the aim of inducing crystallization. After standing at room temperature with occasional shaking for 3.5 hours the sample crystallized as viewed by microscopy. The solids were characterized by ¹H NMR, UPLC, XRPD, DSC,

¹H NMR shows succinate (1.68 equivs), trace IPA (0.037 equivs), UPLC purity 99.2%. XRPD analysis of shows the material to be crystalline (FIG. 5A, Table 8). DSC shows broad endothermic events between 92-129° C. (onset 99° C.) and above 167° C. (likely decomposition) (FIG. 5B). TGA shows 1.9% mass loss between 37-128° C. and decomposition above ca. 167° C. (FIG. 5C).

Similar results were obtained in THE, IPA, and IPA/water mixture.

TABLE 8
Diffraction Peaks for the Succinate Salt of Compound 1

| Peak | Peak | Relative |
|---|---|---|
| number | position °2θ | Intensity |
| Peak 1 | 7.822 | 26.4% |
| Peak 2 | 8.695 | 58.2% |
| Peak 3 | 9.715 | 1.5% |
| Peak 4 | 10.778 | 24.1% |
| Peak 5 | 11.616 | 7.9% |
| Peak 6 | 12.004 | 2.7% |
| Peak 7 | 12.531 | 26.1% |
| Peak 8 | 13.877 | 3.9% |
| Peak 9 | 14.332 | 39.1% |
| Peak 10 | 15.57 | 10.9% |
| Peak 11 | 15.869 | 3.3% |
| Peak 12 | 16.461 | 5.1% |
| Peak 13 | 16.763 | 39.2% |
| Peak 14 | 17.411 | 8.9% |
| Peak 15 | 17.965 | 6.5% |
| Peak 16 | 18.562 | 40.6% |
| Peak 17 | 19.033 | 31.9% |
| Peak 18 | 19.744 | 100.0% |
| Peak 19 | 20.172 | 31.2% |
| Peak 20 | 20.698 | 2.1% |
| Peak 21 | 21.245 | 15.7% |
| Peak 22 | 21.688 | 14.3% |
| Peak 23 | 22.07 | 67.9% |
| Peak 24 | 22.323 | 8.2% |
| Peak 25 | 23.135 | 69.8% |
| Peak 26 | 23.563 | 54.4% |
| Peak 27 | 23.855 | 23.4% |
| Peak 28 | 24.568 | 5.1% |
| Peak 29 | 24.929 | 5.4% |
| Peak 30 | 25.349 | 31.3% |
| Peak 31 | 26.23 | 3.9% |
| Peak 32 | 26.89 | 9.2% |
| Peak 33 | 27.555 | 4.8% |
| Peak 34 | 28.088 | 7.9% |
| Peak 35 | 28.293 | 9.2% |
| Peak 36 | 28.859 | 6.2% |
| Peak 37 | 29.312 | 9.6% |
| Peak 38 | 30.207 | 7.2% |
| Peak 39 | 30.427 | 6.7% |
| Peak 40 | 33.214 | 5.2% |
| Peak 41 | 34.615 | 4.6% |
| Peak 42 | 35.058 | 3.7% |
| Peak 43 | 35.65 | 2.1% |
| Peak 44 | 37.95 | 3.2% |
| Peak 45 | 38.653 | 1.5% |
| Peak 46 | 39.345 | 2.5% |

### B. L-Tartrate Salt of Compound 1

The free base of Compound 1 was weighed into a vial, treated with IPA (125 μL), and briefly shaken to give solutions. L-tartaric acid as a solution in THE was then carefully added to the free base solution.

L-tartrate salt (1 equivalent) showed an amorphous solid by XRPD. ¹H NMR consistent was consistent with the structure (shows tartrate ca. 1.0 equivalent, THE 0.067 equivalents, IPA 0.02 equivalents and small aromatic impurities). A portion of this material (2.0 mg) was treated with IPA (50 μL) and shaken at room temperature. After 1 week some crystalline material was visible by optical microscopy. XRPD analysis showed some weak diffraction peaks (FIG. 6A). Thermal analysis by DSC showed and endothermic event of onset 170° C. (FIG. 6B).

L-tartrate salt (2 equivalents) showed an amorphous solid by XRPD. ¹H NMR was consistent with the structure (shows tartrate ca. 1.0 equivalent, THE 0.21 equivalents, IPA 0.57 equivalents and small aromatic impurities). A portion of this material (2.52 mg) was treated with IPA (50 μL) and shaken at room temperature. After 3 weeks some crystalline material was visible by optical microscopy. XRPD analysis (2 equivalents of L-tartrate) showed some weak diffraction peaks of the same pattern as (1 equivalents of L-tartrate).

TABLE 9
Diffraction Peaks for the Tartrate Salt of Compound 1

| Peak number | Position °2Theta | Relative Intensity |
|---|---|---|
| 1 | 5.529 | 17.2% |
| 2 | 7.45 | 52.6% |
| 3 | 7.983 | 11.1% |
| 4 | 10.478 | 18.3% |
| 5 | 11.258 | 22.0% |
| 6 | 11.645 | 30.0% |
| 7 | 12.434 | 29.2% |
| 8 | 12.672 | 12.0% |
| 9 | 14.623 | 54.1% |
| 10 | 16.035 | 76.1% |
| 11 | 16.456 | 12.0% |
| 12 | 16.839 | 42.2% |
| 13 | 16.849 | 38.0% |
| 14 | 17.486 | 38.3% |
| 15 | 18.858 | 39.5% |
| 16 | 19.57 | 46.9% |
| 17 | 20.069 | 90.2% |
| 18 | 20.551 | 18.4% |
| 19 | 21.153 | 22.5% |
| 20 | 21.663 | 25.7% |
| 21 | 23.387 | 56.6% |
| 22 | 22.981 | 100.0% |
| 23 | 23.804 | 32.5% |
| 24 | 25.501 | 21.0% |
| 25 | 27.474 | 9.4% |
| 26 | 29.065 | 9.7% |
| 27 | 30.776 | 10.4% |
| 28 | 34.793 | 11.3% |

### Example 3. Characterization of the Hydrochloride Salt of Compound 1

The hydrochloride salt of Compound 1 was characterized by XRPD. Analysis by XRPD shows the material to be crystalline (FIGS. 8A-8B, Tables 10-11).

TABLE 10
Diffraction Peaks for the Hydrochloride
Salt of Compound 1 (“Form 10”)

| Peak number | Position °2Theta | Relative Intensity |
|---|---|---|
| Peak 1 | 7.474 | 2.9% |
| Peak 2 | 7.757 | 21.6% |
| Peak 3 | 9.857 | 42.7% |
| Peak 4 | 10.394 | 26.1% |
| Peak 5 | 11.241 | 6.5% |
| Peak 6 | 13.3 | 12.5% |
| Peak 7 | 13.675 | 3.4% |
| Peak 8 | 15.415 | 14.8% |
| Peak 9 | 16.115 | 44.0% |
| Peak 10 | 16.755 | 25.1% |
| Peak 11 | 17.589 | 44.6% |
| Peak 12 | 17.879 | 5.6% |
| Peak 13 | 18.243 | 15.1% |
| Peak 14 | 18.691 | 24.8% |
| Peak 15 | 19.695 | 37.9% |
| Peak 16 | 20.296 | 47.3% |
| Peak 17 | 20.724 | 51.2% |
| Peak 18 | 21.141 | 29.1% |
| Peak 19 | 21.429 | 14.6% |
| Peak 20 | 21.928 | 26.7% |
| Peak 21 | 22.395 | 11.0% |
| Peak 22 | 22.744 | 8.8% |
| Peak 23 | 23.32 | 100.0% |
| Peak 24 | 23.6 | 35.7% |
| Peak 25 | 23.909 | 22.6% |
| Peak 26 | 24.864 | 3.4% |
| Peak 27 | 25.22 | 5.7% |
| Peak 28 | 26.347 | 4.7% |
| Peak 29 | 26.7 | 36.5% |
| Peak 30 | 27.442 | 18.1% |
| Peak 31 | 28.161 | 28.1% |
| Peak 32 | 28.946 | 18.8% |
| Peak 33 | 29.712 | 2.1% |
| Peak 34 | 30.96 | 18.7% |
| Peak 35 | 31.467 | 8.3% |
| Peak 36 | 32.5 | 4.6% |
| Peak 37 | 32.96 | 4.2% |
| Peak 38 | 33.464 | 5.2% |
| Peak 39 | 33.812 | 7.6% |
| Peak 40 | 35.31 | 4.3% |
| Peak 41 | 36.152 | 4.0% |
| Peak 42 | 36.675 | 6.5% |
| Peak 43 | 37.126 | 6.7% |
| Peak 44 | 37.908 | 4.0% |
| Peak 45 | 38.752 | 4.0% |
| Peak 46 | 39.338 | 4.7% |
| Peak 47 | 39.965 | 3.1% |
| Peak 48 | 40.834 | 2.7% |

TABLE 11
Diffraction Peaks for the Hydrochloride
Salt of Compound 1 (“Form 11”)

| Peak number | Position °2Theta | Relative Intensity |
|---|---|---|
| 1 | 7.617 | 5.2% |
| 2 | 7.886 | 21.3% |
| 3 | 9.806 | 34.8% |
| 4 | 10.65 | 13.6% |
| 5 | 10.915 | 13.8% |
| 6 | 13.093 | 37.7% |
| 7 | 13.674 | 3.4% |
| 8 | 14.341 | 12.1% |
| 9 | 14.608 | 4.7% |
| 10 | 15.697 | 19.3% |
| 11 | 15.898 | 8.7% |
| 12 | 16.338 | 46.0% |
| 13 | 16.668 | 17.9% |
| 14 | 17.229 | 34.2% |
| 15 | 18.807 | 45.0% |
| 16 | 19.602 | 5.0% |
| 17 | 19.858 | 10.1% |
| 18 | 20.215 | 47.3% |
| 19 | 20.405 | 37.8% |
| 20 | 20.767 | 35.7% |
| 21 | 21.076 | 22.7% |
| 22 | 21.357 | 33.1% |
| 23 | 22.034 | 47.2% |
| 24 | 22.698 | 100.0% |
| 25 | 22.949 | 24.0% |
| 26 | 23.885 | 25.6% |
| 27 | 24.476 | 29.5% |
| 28 | 24.881 | 19.8% |
| 29 | 26.309 | 10.8% |
| 30 | 27.004 | 37.0% |
| 31 | 27.237 | 6.4% |
| 32 | 27.842 | 13.2% |
| 33 | 28.249 | 18.2% |
| 34 | 28.431 | 9.5% |
| 35 | 29.338 | 4.4% |
| 36 | 29.494 | 8.8% |
| 37 | 30.608 | 10.2% |
| 38 | 31.727 | 6.5% |
| 39 | 32.004 | 12.3% |
| 40 | 32.864 | 7.0% |
| 41 | 34.342 | 5.1% |
| 42 | 35.7 | 4.0% |
| 43 | 36.583 | 2.2% |
| 44 | 37.202 | 4.2% |
| 45 | 37.68 | 3.6% |
| 46 | 37.938 | 5.8% |
| 47 | 38.836 | 3.6% |
| 48 | 40.542 | 3.2% |

### Example 4: Characterization of the Pyruvate Salt of Compound 1

Pyruvate salt of Compound 1 was characterized by XRPD. Analysis by XRPD shows the material to be crystalline (FIG. 9, Table 12).

TABLE 12
Diffraction Peaks for the Pyruvate Salt of Compound 1.

| Peak number | Position °2Theta | Relative Intensity |
|---|---|---|
| 1 | 7.699 | 32.9% |
| 2 | 8.027 | 3.8% |
| 3 | 8.509 | 0.7% |
| 4 | 9.424 | 53.7% |
| 5 | 10.961 | 9.9% |
| 6 | 11.325 | 6.3% |
| 7 | 12.024 | 0.6% |
| 8 | 12.881 | 4.5% |
| 9 | 13.947 | 24.5% |
| 10 | 14.949 | 7.4% |
| 11 | 15.466 | 5.5% |
| 12 | 15.85 | 5.0% |
| 13 | 16.856 | 4.1% |
| 14 | 17.185 | 11.8% |
| 15 | 17.605 | 12.5% |
| 16 | 18.382 | 1.5% |
| 17 | 18.764 | 100.0% |
| 18 | 19.412 | 23.7% |
| 19 | 20.09 | 6.0% |
| 20 | 20.607 | 9.0% |
| 21 | 21.078 | 24.0% |
| 22 | 21.454 | 15.7% |
| 23 | 22.034 | 76.5% |
| 24 | 22.672 | 23.8% |
| 25 | 23.181 | 12.5% |
| 26 | 23.644 | 3.2% |
| 27 | 24.147 | 3.5% |
| 28 | 24.343 | 13.2% |
| 29 | 24.989 | 16.5% |
| 30 | 25.649 | 1.9% |
| 31 | 25.877 | 4.5% |
| 32 | 26.399 | 8.5% |
| 33 | 26.816 | 2.8% |
| 34 | 27.986 | 4.7% |
| 35 | 28.289 | 5.0% |
| 36 | 28.798 | 1.8% |
| 37 | 29.284 | 1.4% |
| 38 | 29.749 | 1.5% |
| 39 | 30.212 | 2.1% |
| 40 | 30.743 | 1.1% |
| 41 | 32.267 | 1.1% |
| 42 | 32.591 | 2.9% |
| 43 | 32.938 | 4.3% |
| 44 | 33.488 | 5.1% |
| 45 | 34.203 | 2.0% |
| 46 | 34.64 | 2.0% |
| 47 | 35.043 | 1.3% |
| 48 | 35.461 | 2.3% |
| 49 | 36.451 | 1.3% |
| 50 | 36.992 | 0.7% |
| 51 | 37.448 | 2.7% |
| 52 | 39.048 | 1.0% |
| 53 | 39.665 | 1.0% |

### Example 5: Characterization of the 3-Oxodipropanoate Salt of Compound 1

3-oxodipropanoate salt of Compound 1 was characterized by XRPD. Analysis by XRPD shows the material to be crystalline (FIGS. 10A-10B, Tables 13-14).

TABLE 13
Diffraction Peaks for the 3-oxodipropanoate
Salt of Compound 1 (“Form 12”).

| Peak number | Position °2Theta | Relative Intensity |
|---|---|---|
| 1 | 7.217 | 24.8% |
| 2 | 7.703 | 17.9% |
| 3 | 9.337 | 50.0% |
| 4 | 10.791 | 28.5% |
| 5 | 11.127 | 45.2% |
| 6 | 13.03 | 28.2% |
| 7 | 14.015 | 25.1% |
| 8 | 14.562 | 10.5% |
| 9 | 14.763 | 13.4% |
| 10 | 15.392 | 17.9% |
| 11 | 16.378 | 31.0% |
| 12 | 16.571 | 24.9% |
| 13 | 16.749 | 14.9% |
| 14 | 18.019 | 100.0% |
| 15 | 18.515 | 23.9% |
| 16 | 18.706 | 14.2% |
| 17 | 19.313 | 61.1% |
| 18 | 20.016 | 24.2% |
| 19 | 20.975 | 23.0% |
| 20 | 21.134 | 26.2% |
| 21 | 21.415 | 18.8% |
| 22 | 21.694 | 77.4% |
| 23 | 22.044 | 13.1% |
| 24 | 22.49 | 3.0% |
| 25 | 23.227 | 89.8% |
| 26 | 23.498 | 21.5% |
| 27 | 23.709 | 20.5% |
| 28 | 24.674 | 4.3% |
| 29 | 24.849 | 6.5% |
| 30 | 25.777 | 6.7% |
| 31 | 26.206 | 6.1% |
| 32 | 28.133 | 6.0% |
| 33 | 28.448 | 9.6% |
| 34 | 29.841 | 5.2% |
| 35 | 30.335 | 4.4% |
| 36 | 30.477 | 6.4% |
| 37 | 32.6 | 6.1% |
| 38 | 33.133 | 4.0% |
| 39 | 33.382 | 4.7% |
| 40 | 33.73 | 3.5% |
| 41 | 34.434 | 3.5% |
| 42 | 37.865 | 3.5% |

TABLE 14
Diffraction Peaks for the 3-Oxodipropanoate
Salt of Compound 1 (“Form 13”).

| Peak number | Position °2Theta | Relative Intensity |
|---|---|---|
| 1 | 7.519 | 23.1% |
| 2 | 7.806 | 20.6% |
| 3 | 8.342 | 1.7% |
| 4 | 9.691 | 1.6% |
| 5 | 10.46 | 4.6% |
| 6 | 11.784 | 1.5% |
| 7 | 12.442 | 2.8% |
| 8 | 12.984 | 1.9% |
| 9 | 13.516 | 4.5% |
| 10 | 13.838 | 14.1% |
| 11 | 14.899 | 31.4% |
| 12 | 16.118 | 21.1% |
| 13 | 16.518 | 7.7% |
| 14 | 17.418 | 1.7% |
| 15 | 17.878 | 3.0% |
| 16 | 18.893 | 5.8% |
| 17 | 19.305 | 100.0% |
| 18 | 20.056 | 7.1% |
| 19 | 20.565 | 13.5% |
| 20 | 21.798 | 62.9% |
| 21 | 22.34 | 50.7% |
| 22 | 23.001 | 11.4% |
| 23 | 23.396 | 2.4% |
| 24 | 24.258 | 11.3% |
| 25 | 24.839 | 11.5% |
| 26 | 25.785 | 8.3% |
| 27 | 26.321 | 5.7% |
| 28 | 27.044 | 2.3% |
| 29 | 27.678 | 3.1% |
| 30 | 28.543 | 1.4% |
| 31 | 28.963 | 3.2% |
| 32 | 29.378 | 4.3% |
| 33 | 30.342 | 2.4% |
| 34 | 32.3 | 2.1% |
| 35 | 34.243 | 1.2% |
| 36 | 34.797 | 1.1% |
| 37 | 39.406 | 1.8% |

### Example 6: Characterization of the Adipate Salt of Compound 1

Adipate salt of Compound 1 was characterized by XRPD. Analysis by XRPD shows the material to be crystalline (FIGS. 11A-11C, Tables 15-17).

TABLE 15
Diffraction Peaks for the Adipate Salt
of Compound 1 (“Form 14”).

| Peak number | Position °2Theta | Relative Intensity |
|---|---|---|
| 1 | 7.504 | 71.5% |
| 2 | 8.576 | 2.6% |
| 3 | 9.264 | 26.1% |
| 4 | 11.308 | 19.4% |
| 5 | 12.568 | 13.0% |
| 6 | 14.205 | 27.8% |
| 7 | 14.954 | 10.5% |
| 8 | 15.253 | 21.1% |
| 9 | 16.26 | 3.1% |
| 10 | 16.738 | 11.3% |
| 11 | 17.059 | 8.1% |
| 12 | 18.024 | 100.0% |
| 13 | 18.541 | 62.2% |
| 14 | 19.292 | 16.0% |
| 15 | 20.764 | 5.1% |
| 16 | 21.218 | 40.2% |
| 17 | 21.671 | 12.4% |
| 18 | 22.334 | 37.8% |
| 19 | 22.845 | 4.3% |
| 20 | 23.399 | 18.8% |
| 21 | 23.773 | 7.8% |
| 22 | 23.897 | 14.4% |
| 23 | 24.344 | 9.3% |
| 24 | 25.058 | 11.7% |
| 25 | 25.836 | 4.8% |
| 26 | 27.018 | 1.9% |
| 27 | 28.09 | 5.8% |
| 28 | 28.597 | 5.5% |
| 29 | 29.78 | 3.1% |
| 30 | 30.813 | 5.6% |
| 31 | 33.686 | 2.6% |
| 32 | 34.753 | 1.1% |
| 33 | 35.494 | 1.0% |
| 34 | 37.363 | 1.4% |
| 35 | 40.12 | 1.3% |

TABLE 16
Diffraction Peaks for the Adipate Salt
of Compound 1 (“Form 15”).

| Peak number | Position °2Theta | Relative Intensity |
|---|---|---|
| 1 | 7.444 | 91.1% |
| 2 | 8.973 | 67.3% |
| 3 | 9.252 | 6.9% |
| 4 | 9.773 | 4.9% |
| 5 | 10.972 | 21.3% |
| 6 | 11.225 | 8.2% |
| 7 | 12.125 | 20.3% |
| 8 | 12.529 | 23.2% |
| 9 | 13.143 | 13.9% |
| 10 | 13.44 | 5.8% |
| 11 | 14.191 | 100.0% |
| 12 | 14.621 | 11.5% |
| 13 | 15.124 | 51.1% |
| 14 | 15.798 | 13.2% |
| 15 | 16.786 | 23.8% |
| 16 | 17.272 | 40.1% |
| 17 | 17.624 | 19.6% |
| 18 | 18.014 | 54.4% |
| 19 | 18.545 | 17.3% |
| 20 | 18.774 | 23.3% |
| 21 | 19.068 | 31.3% |
| 22 | 19.302 | 40.6% |
| 23 | 19.67 | 48.4% |
| 24 | 19.926 | 48.6% |
| 25 | 20.391 | 15.8% |
| 26 | 20.71 | 14.0% |
| 27 | 21.236 | 8.9% |
| 28 | 21.516 | 16.7% |
| 29 | 21.959 | 23.9% |
| 30 | 22.336 | 23.0% |
| 31 | 22.777 | 30.9% |
| 32 | 23.31 | 59.2% |
| 33 | 23.622 | 21.8% |
| 34 | 24.209 | 9.9% |
| 35 | 24.816 | 35.3% |
| 36 | 24.98 | 6.4% |
| 37 | 25.965 | 9.0% |
| 38 | 26.425 | 30.2% |
| 39 | 27.027 | 9.2% |
| 40 | 27.431 | 5.8% |
| 41 | 28.575 | 9.1% |
| 42 | 29.863 | 3.3% |
| 43 | 31.236 | 10.0% |
| 44 | 32.281 | 4.4% |
| 45 | 32.736 | 2.9% |
| 46 | 33.359 | 4.1% |
| 47 | 33.691 | 3.9% |
| 48 | 37.548 | 4.4% |
| 49 | 38.606 | 3.1% |

TABLE 17
Diffraction Peaks for the Adipate Salt
of Compound 1 (“Form 16”).

| Peak number | Position °2Theta | Relative Intensity |
|---|---|---|
| 1 | 7.468 | 100.0% |
| 2 | 9.27 | 2.6% |
| 3 | 10.952 | 10.4% |
| 4 | 11.198 | 6.8% |
| 5 | 12.116 | 1.7% |
| 6 | 12.535 | 28.3% |
| 7 | 14.161 | 10.6% |
| 8 | 14.641 | 9.8% |
| 9 | 15.221 | 6.7% |
| 10 | 15.813 | 20.5% |
| 11 | 16.809 | 25.5% |
| 12 | 17.567 | 58.6% |
| 13 | 17.774 | 36.2% |
| 14 | 18.001 | 14.6% |
| 15 | 18.551 | 8.0% |
| 16 | 19.151 | 20.1% |
| 17 | 19.963 | 49.2% |
| 18 | 20.374 | 6.8% |
| 19 | 20.657 | 13.5% |
| 20 | 21.175 | 6.6% |
| 21 | 21.976 | 38.7% |
| 22 | 22.274 | 7.7% |
| 23 | 22.803 | 50.5% |
| 24 | 23.183 | 8.3% |
| 25 | 23.658 | 11.4% |
| 26 | 23.869 | 17.8% |
| 27 | 24.407 | 11.6% |
| 28 | 24.932 | 7.7% |
| 29 | 25.314 | 4.5% |
| 30 | 25.581 | 2.9% |
| 31 | 25.868 | 10.5% |
| 32 | 26.812 | 4.9% |
| 33 | 27.41 | 11.3% |
| 34 | 28.988 | 3.1% |
| 35 | 29.822 | 3.0% |
| 36 | 30.855 | 4.2% |
| 37 | 31.398 | 1.9% |
| 38 | 32.369 | 1.1% |
| 39 | 35.479 | 1.8% |
| 40 | 36.104 | 1.6% |
| 41 | 37.472 | 2.0% |
| 42 | 39.033 | 1.7% |

### Example 7: Characterization of the Oxalate Salt of Compound 1

Oxalate salt of Compound 1 was characterized by XRPD. Analysis by XRPD shows the material to be crystalline (FIG. 12, Table 18).

TABLE 18
Diffraction Peaks for the Oxalate Salt of Compound 1.

| Peak number | Position °2Theta | Relative Intensity |
|---|---|---|
| 1 | 8.887 | 4.7% |
| 2 | 11.551 | 12.3% |
| 3 | 13.432 | 44.4% |
| 4 | 13.903 | 100.0% |
| 5 | 14.299 | 56.7% |
| 6 | 16.323 | 40.8% |
| 7 | 17.092 | 51.4% |
| 8 | 18.104 | 33.5% |
| 9 | 19.436 | 56.1% |
| 10 | 19.276 | 17.1% |
| 11 | 20.178 | 28.5% |
| 12 | 21.53 | 33.0% |
| 13 | 21.876 | 93.6% |
| 14 | 23.052 | 28.3% |
| 15 | 24.139 | 28.1% |
| 16 | 24.76 | 69.7% |
| 17 | 25.047 | 34.4% |
| 18 | 26.733 | 13.6% |
| 19 | 27.564 | 57.0% |
| 20 | 34.357 | 16.4% |
| 21 | 34.76 | 11.8% |

1. A pharmaceutically acceptable salt of Compound 1

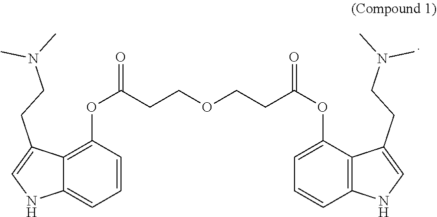

2. The pharmaceutically acceptable salt of claim 1, wherein the salt is a monofumarate salt of Compound 1, a sesquifumarate salt of Compound 1, succinate salt of Compound 1, L-tartrate salt of Compound 1, hydrochloride salt of Compound 1, pyruvate salt of Compound 1, 3-oxodipropanoate salt of Compound 1, adipate salt of Compound 1, or oxalate salt of Compound 1.

3. The pharmaceutically acceptable salt of claim 2, wherein the salt is a monofumarate salt of Compound 1.

4. The pharmaceutically acceptable salt of claim 1, wherein the salt is a sesquifumarate salt of Compound 1.

5. The pharmaceutically acceptable salt of claim 2, wherein the salt is a succinate salt of Compound 1.

6. The pharmaceutically acceptable salt of claim 2, wherein the salt is a L-tartrate salt of Compound 1.

7. The pharmaceutically acceptable salt of claim 2, wherein the salt is a hydrochloride salt of Compound 1.

8. The pharmaceutically acceptable salt of claim 2, wherein the salt is a pyruvate salt of Compound 1.

9. The pharmaceutically acceptable salt of claim 2, wherein the salt is a 3-oxodipropanoate salt of Compound 1.

10. The pharmaceutically acceptable salt of claim 2, wherein the salt is an adipate salt of Compound 1.

11. The pharmaceutically acceptable salt of claim 2, wherein the salt is an oxalate salt of Compound 1.

12. The pharmaceutically acceptable salt of claim 3, wherein the monofumarate salt of Compound 1 is crystalline.

13-57. (canceled)

58. The pharmaceutically acceptable salt of claim 4, wherein the sesquifumarate salt of Compound 1 is crystalline.

59. The pharmaceutically acceptable salt of claim 58, characterized by an XRPD pattern having peaks at 8.7±0.2, 19.7±0.2, and 23.6±0.2 °2θ.

60. The pharmaceutically acceptable salt of claim 59, further characterized by peaks in an XRPD pattern at 14.4±0.2, 21.8±0.2, and 22.2±0.2 °2θ.

61. The pharmaceutically acceptable salt of claim 58, characterized by an XRPD pattern having peaks at 7.9±0.2, 8.7±0.2, 9.6±0.2, 11.1±0.2, 11.6±0.2, 12.3±0.2, 14.0±0.2, 14.4±0.2, 15.6±0.2, 16.3±0.2, 16.8±0.2, 17.2±0.2, 17.4±0.2, 17.8±0.2, 18.0±0.2, 18.4±0.2, 18.7±0.2, 19.2±0.2, 19.7±0.2, 20.1±0.2, 20.8±0.2, 21.1±0.2, 21.4±0.2, 21.8±0.2, 22.2±0.2, 22.5±0.2, 22.9±0.2, 23.2±0.2, 23.6±0.2, 24.3±0.2, 24.6±0.2, 25.3±0.2, 26.4±0.2, 26.8±0.2, 27.4±0.2, 28.2±0.2, 29.0±0.2, 29.5±0.2, 29.7±0.2, 30.1±0.2, 30.4±0.2, 30.9±0.2, 31.6±0.2, 33.2±0.2, 34.0±0.2, 34.7±0.2, 34.9±0.2, 35.4±0.2, 38.4, 39.0±0.2, and 39.9±0.2 and °2θ.

62. The pharmaceutically acceptable salt of claim 5826, characterized by an XRPD pattern substantially similar to FIG. 4A.

63. The pharmaceutically acceptable salt of claim 58, characterized by a DSC thermogram comprising an endothermic event at 168±5° C.

64. The pharmaceutically acceptable salt of claim 58, characterized by a DSC comprising an exothermic event between 176±5° C. to 221±5° C.

65. The pharmaceutically acceptable salt of claim 58, characterized by a DSC thermogram substantially similar to FIG. 4C.

66. The pharmaceutically acceptable salt of claim 58, characterized by about a 2% weight loss from 37±5° C. to 150±5° C. as determined by TGA.

67. The pharmaceutically acceptable salt of claim 58, characterized by a thermogravimetric thermogram substantially similar to FIG. 4D.

68. The pharmaceutically acceptable salt of claim 58, having a chemical purity of greater than about 90%, by weight, as determined by HPLC analysis.

69. The pharmaceutically acceptable salt of claim 58, having a chemical purity of greater than about 95%, by weight, as determined by HPLC analysis.

70. The pharmaceutically acceptable salt of claim 58, having a chemical purity of greater than about 99.5%, by weight, as determined by HPLC analysis.

71. The pharmaceutically acceptable salt of claim 58, wherein the sesquifumarate salt of Compound 1 is a hydrate.

72-151. (canceled)

152. A pharmaceutical composition comprising a pharmaceutically acceptable salt of Compound 1 of claim 1.

153. A fumarate salt of Compound 1: 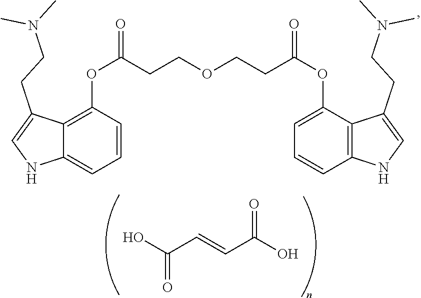
wherein n is from 1 to 3.

154. The fumarate salt of claim 153, wherein the fumarate salt is crystalline.

155. The fumarate salt of claim 153, having the following formula:

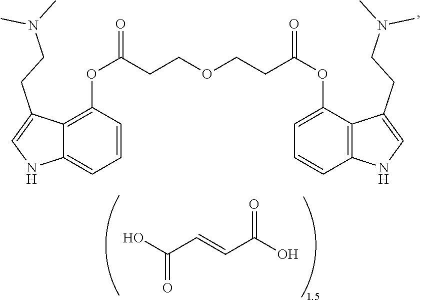

156. The fumarate salt of claim 155, characterized by an X-ray powder diffraction (XRPD) pattern having peaks at 8.7±0.2, 19.7±0.2, and 23.6±0.2 °2θ.

157. The fumarate salt of claim 156, further characterized by peaks in an XRPD pattern at 14.4±0.2, 21.8±0.2, and 22.2±0.2 °2θ.

158. The fumarate salt of claim 155, characterized by an XRPD pattern having peaks at 7.9±0.2, 8.7±0.2, 9.6±0.2, 11.1±0.2, 11.6±0.2, 12.3±0.2, 14.0±0.2, 14.4±0.2, 15.6±0.2, 16.3±0.2, 16.8±0.2, 17.2±0.2, 17.4±0.2, 17.8±0.2, 18.0±0.2, 18.4±0.2, 18.7±0.2, 19.2±0.2, 19.7±0.2, 20.1±0.2, 20.8±0.2, 21.1±0.2, 21.4±0.2, 21.8±0.2, 22.2±0.2, 22.5±0.2, 22.9±0.2, 23.2±0.2, 23.6±0.2, 24.3±0.2, 24.6±0.2, 25.3±0.2, 26.4±0.2, 26.8±0.2, 27.4±0.2, 28.2±0.2, 29.0±0.2, 29.5±0.2, 29.7±0.2, 30.1±0.2, 30.4±0.2, 30.9±0.2, 31.6±0.2, 33.2±0.2, 34.0±0.2, 34.7±0.2, 34.9±0.2, 35.4±0.2, 38.4, 39.0±0.2, and 39.9±0.2 and °2θ.

159. The fumarate salt of claim 153, having the following formula:

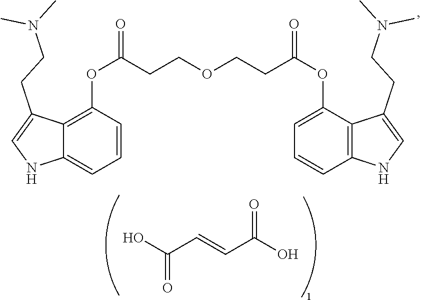

160. The salt of claim 159, characterized by an XRPD pattern having peaks at 12.8±0.2, 18.7±0.2, and 23.2±0.2 °2θ.

161. The salt of claim 160, further characterized by peaks in an XRPD pattern at 7.2±0.2, 10.4±0.2, 17.9±0.2, 19.3±0.2, and 21.4±0.2 °2θ.

162. The salt of claim 159, characterized by an XRPD pattern having peaks at 5.3±0.2, 7.2±0.2, 7.7±0.2, 8.9±0.2, 10.4±0.2, 11.3±0.2, 12.8±0.2, 13.7±0.2, 14.4±0.2, 14.7±0.2, 15.4±0.2, 16.5±0.2, 16.9±0.2, 17.3±0.2, 17.9±0.2, 18.7±0.2, 19.3±0.2, 19.8±0.2, 20.8±0.2, 21.0±0.2, 21.4±0.2, 21.8±0.2, 22.8±0.2, 23.2±0.2, 24.5±0.2, 24.8±0.2, 25.7±0.2, 26.4±0.2, 27.4±0.2, 28.6±0.2, 29.0±0.2, 29.5±0.2, 30.2±0.2, 30.6±0.2, 31.3±0.2, 32.3±0.2, and 32.7±0.2 °2θ.

163. A succinate salt of Compound 1: 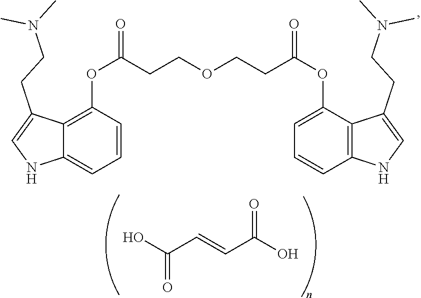
wherein n is from 1 to 2.

164. A hydrochloride salt of Compound 1: 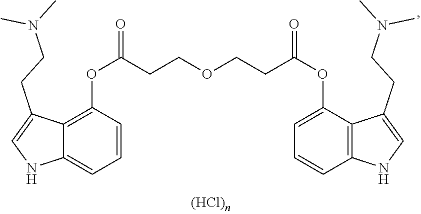
wherein n is from 1 to 2.

165. A pyruvate salt of Compound 1: 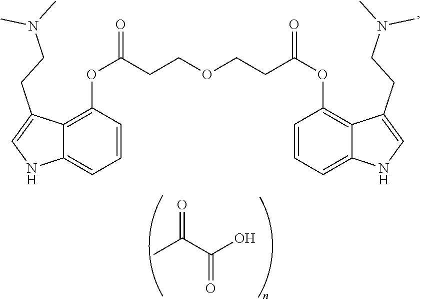
wherein n is from 1 to 2.

166. A 3-oxodipropanoate salt of Compound 1: 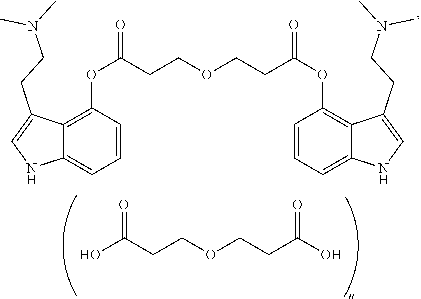
wherein n is from 1 to 2.

167. An adipate salt of Compound 1: 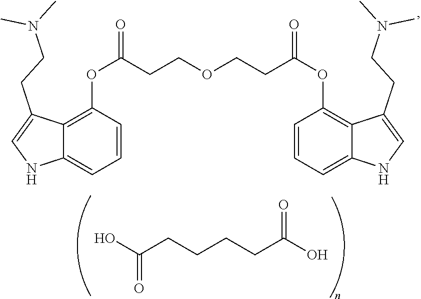
wherein n is from 1 to 3.

168. An oxalate salt of Compound 1: 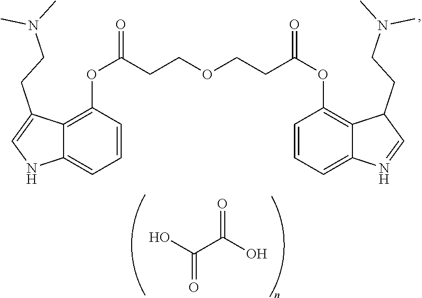
wherein n is from 1 to 2.

169. A compound, having the following structure (A):

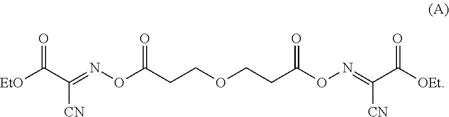 [SMILES: CCOC(=O)/C(C#N)=N/OC(=O)CCOCCC(=O)O/N=C(\C#N)C(=O)OCC]

170. A method of manufacturing the compound of structure (A) of claim 169, comprising:
(i) reacting 3,3′-oxydipropionic acid with thionyl dichloride to form 3,3′-oxydipropionyl dichloride; and
(ii) reacting 3,3′-oxydipropionyl dichloride with ethyl 2-cyano-2-(hydroxyimino)acetate (bis-oxyma) in the presence of a base and a solvent to form the compound of structure (A).

171. The method of claim 170, wherein the base in (ii) is triethylamine (TEA).

172. The method of claim 170, wherein the solvent in (ii) is ethyl acetate (EtOAc).

173. A method of manufacturing the pharmaceutically acceptable salt of Compound 1 of claim 1, comprising:
(i) reacting the compound of structure (A) with psilocin in the presence of a base and a solvent to form free base Compound 1; and
(ii) reacting the free base Compound 1 with an acid to form the pharmaceutically acceptable salt.

174. The method of claim 173, wherein the base in (i) is TEA.

175. The method of claim 173, wherein the solvent in (i) EtOAc.

176. The method of claim 173, wherein the reacting in (ii) is performed in the presence of a solvent.

177. The method of claim 176, wherein the solvent is a mixture of isopropyl alcohol and water.

178. The method of claim 173, further comprising (iii) recrystallizing the pharmaceutically acceptable salt.

179. The method of claim 173, wherein the acid is monofumaric acid, sesquifumaric acid, succinic acid, L-tartric acid, hydrochloric acid, pyruvic acid, oxodipropanoic acid, adipate salt or oxalic acid.

180. The method of claim 173, wherein the psilocin is prepared by a method comprising:
(i) reacting 4-acetoxyindole with oxalyl chloride to form 3-(2-chloro-2-oxoacetyl)-1H-indol-4-yl acetate;
(ii) reacting 3-(2-chloro-2-oxoacetyl)-1H-indol-4-yl acetate with dimethylamine to form 3-([(dimethylcarbamoyl)carbonyl])-1H-indol-4-yl acetate; and
(iii) reacting 3-([(dimethylcarbamoyl)carbonyl])-1H-indol-4-yl acetate with lithium aluminum hydride to form the psilocin.

181. The method of claim 180, wherein the reacting in (ii) is performed in the presence of TEA.
